(19) United States
(12) Patent Application Publication
(10) Pub. No.: US 2014/0378408 A1
(43) Pub. Date: Dec. 25, 2014

(54) NUCLEOSIDE 5'-PHOSPHOROTHIOATE ANALOGUES AND USES THEREOF

(71) Applicants: BAR-ILAN UNIVERSITY, an Israeli University, Ramat-Gan (IL); The Medical Research, Infrastructure, and Health Services Fund of the Tel-Aviv Medical Center, Tel-Aviv (IL)

(72) Inventors: Bilha FISCHER, Shoham (IL); Ortal SHIMON, Gedera (IL); Yael NADEL, Holon (IL); Uri ARAD, Tel-Aviv (IL)

(21) Appl. No.: 14/479,181

(22) Filed: Sep. 5, 2014

Related U.S. Application Data
(63) Continuation-in-part of application No. PCT/IL2013/050202, filed on Mar. 5, 2013.
(60) Provisional application No. 61/634,698, filed on Mar. 5, 2012.

(51) Int. Cl. C07H 19/20

(52) U.S. Cl. CPC C07H 19/20
USPC 514/47; 536/26.26; 536/26.23; 536/26.22

(57) ABSTRACT

Particular nucleoside 5′-phosphorothioate analogues, such as, adenosine or uridine 5′-di- or tri-phosphorothioate analogues in which at least one of the bridging oxygen atoms of the phosphorothioate is replaced by a group such as —CH₂— or —CCl₂—, and at least one of the non-bridging atoms or negatively-charged atoms of the phosphorothioate is either a sulfur atom or a sulfur ion can be formulated into pharmaceutical compositions. These compounds are useful for treatment of osteoarthritis/calcium pyrophosphate dihydrate (CPPD) deposition disease.

### CROSS-REFERENCE TO RELATED APPLICATIONS

The present invention is a continuation-in-part application of International Application No. PCT/IL2013/050202, filed Mar. 5, 2013, and claims the benefit of U.S. Provisional Patent Application No. 61/634,698, filed Mar. 5, 2012, now expired, the entire content of each and all these applications being herewith incorporated by reference in their entirety as if fully disclosed herein.

### TECHNICAL FIELD

The present invention provides nucleoside 5′-phosphorothioate analogues as well as pharmaceutical compositions thereof, which are useful, inter alia, for treatment of osteoarthritis/calcium pyrophosphate dihydrate (CPPD) disease.

Abbreviations: AD, Alzheimer's disease; ADP, adenosine diphosphate; AMP, adenosine monophosphate; APCPP-γ-S, adenosine 5′-[γ-thio]-α,β-methylene triphosphate; APPCP-α-S, adenosine 5′-[α-thio]-β,γ-methylene triphosphate; ATP, adenosine triphosphate; BBB, blood brain barrier; BCA, bicinchoninic acid; [Ca²⁺]ᵢ, intracellular Ca²⁺ concentration; CDI, carbodiimidazole; Clioquinol, 5-chloro-7-iodoquinolin-8-ol (CQ); CNS, central nervous system; CPPD, calcium pyrophosphate dihydrate; DBU, 1,8-diazabicyclo[5.4.0]undec-7-ene; DCM, dichloromethane; DLS, dynamic light scattering; DMAP, 4-dimethylaminopyridine; DMEM, Dulbecco's modified Eagles' medium; DMF, N,N-dimethylformamide; DMPO, 5,5′-dimethyl-1-pyrroline-N-oxide; DMSO, dimethyl sulfoxide; EDTA, ethylenediamine tetraacetic acid; ESI, electrospray ionization; ESR, electron spin resonance; FTIR, fourier transform infrared spectroscopy: GDP, gunosine diphosphate; GFP, green fluorescent protein; GLUT1, glucose transporter 1; GSH, glutathione; GTP, guanosine triphosphate; HPLC, high-pressure liquid chromatography; LC, liquid chromatography; MDPT, methylene diphosphonotetrathioic acid; MTT, dimethylthiazol-2-yl)-2,5-diphenyl tetrazolium bromide; NPP, nucleotide pyrophosphatase/phosphodiesterase; NTPDase, nucleoside triphosphate diphosphohydrolase; PBS, phosphate buffered saline; pnp-TMP, thymidine 5′-monophosphate p-nitrophenyl ester; ROS, reactive oxygen species; RT, room temperature; TBA, tert-butyl alcohol; TEAA, triethylammonium acetate; TEAB, uriethylammonium bicarbonate; TEM, transition electron microscopy; TFA, trifluoroacetic acid; THF, tetrahydrofuran; TLC, thin layer chromatography.

### BACKGROUND ART

Osteoarthritis (OA) and calcium pyrophosphate dihydrate (CPPD) deposition disease (chondrocalcinosis) are joint pathologies characterized by gradually developing symptoms involving pain, stiffness and loss of joints function. As these diseases have no cure, they have a major impact on life quality and productivity, resulting in a significant socio-economic burden. Recent studies have established a relationship between CPPD crystal deposition at the joints and the pathogenesis of osteoarthritis. CPPD crystals are produced from calcium ions and extracellular pyrophosphate. The latter is produced from ATP hydrolysis by ecto-nucleotidase NPP1 (eNPP1). Based on this mechanism, NPP1 inhibitors were suggested as potential therapeutic agents for the treatment of CPPD and OA.

Osteoarthritis is the most common type of arthritis or degenerative joint disease. It is a leading cause of chronic disability. The disease most commonly affects the middle-aged and elderly, although younger people may be affected as a result of sport injury or overuse. Age is the strongest predictor of the disease and therefore increasing age and extended life expectancy will result in a greater occurrence of the disease. In addition, a rapid increase in the obese population is also expected to contribute to the increase of the incidence of osteoarthritis.

The current treatment options are moderately successful in meeting the market demand. Current treatments are limited to NSAIDS, COX-2 inhibitors, opioid analgesics and joint replacement surgery. There is no cure or preventive treatment, a number of prospective disease-modifying osteoarthritis drugs (DMOAD) are under investigation and some of those are in advanced clinical development (Barr and Conaghan, 2013).

There are a number of prospective targets for structural and symptomatic disease modification in patients with established osteoarthritis, early osteoarthritis, or at the time of acute joint injury, with a view to preventing structural progression, improving symptoms and function, and avoiding the need for total joint replacement. Disease modifying treatments are the highest unmet need and prospective therapies will need to demonstrate excellent safety profiles in view of their target population.

U.S. Pat. No. 7,368,439 discloses diribo-, di-2′-deoxyribo, and ribo-2′-deoxyribo-nucleoside boranophosphate derivatives that can be useful for prevention or treatment of diseases or disorders modulated by P2Y receptors such as type 2 diabetes, cystic fibrosis and cancer. WO 2009/066298 discloses non-hydrolyzable adenosine and uridine polyphosphate derivatives, said to be useful for prevention or treatment of diseases modulated by P2Y-receptors such as type 2 diabetes. WO 2011/077435 discloses ophthalmic compositions for reducing intraocular pressure, comprising a non-hydrolyzable nucleoside di- or triphosphate analogue in which the α,β- or β,γ-bridging-oxygen, respectively, is replaced with, e.g., a methylene or dihalomethylene group. WO 2012/032513 discloses pharmaceutical compositions for treatment and management of osteoarthritis, comprising either a dinucleotide boranophosphate derivative or a nucleoside boranophosphate derivative, in which at least one of the bridging-oxygens in the dinucleoside boranophosphate derivative, preferably both the α,β- and δ,ε-bridging-oxygens, and at least one of the bridging-oxygens in the nucleoside boranophosphate derivative, each is replaced with a group selected from —NH— or —C(R₁₀R₁₁)—, wherein R₁₀ and R₁₁ each independently is H or halogen. WO 2012/073237 discloses uridine nucleotides in which the carbon atom at position 5 of the uracil ring is substituted by —O-alkyl or —S-alkyl, and at least one of the non-bridging oxygen atoms of the di- or tri-phosphate is replaced by a borano group, which can be useful for treatment of diseases, disorders and conditions modulated by P2Y₆ receptors, particularly for lowering intraocular pressure. All these publications, based on studies conducted in the laboratories of the present inventors, are herewith incorporated by reference in their entirety as if fully described herein.

### SUMMARY OF INVENTION

In one aspect, the present invention provides a compound, more particularly a nucleoside 5′-phosphorothioate, of the general formula I:

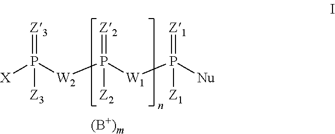

or a diastereomer or mixture of diastereomers thereof,

wherein

X is —O⁻, a glucose moiety linked through the oxygen atom linked to its 1- or 6-position, or a group of the formula —O—CH₂—OC(O)—R₁₂ or —NH—(CHR₁₃)—C(O)—OR₁₃;

Nu is an adenosine residue of the formula Ia, linked through the oxygen atom linked to the 5′-position:

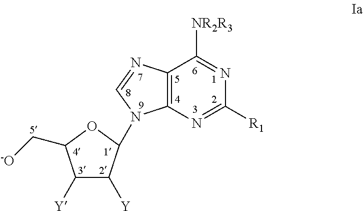

wherein

R₁ is H, halogen, —O-hydrocarbyl, —S-hydrocarbyl, —NR₄R₅, heteroaryl, or hydrocarbyl optionally substituted by one or more groups each independently selected from halogen, —CN, —SCN, —NO₂, —OR₄, —SR₄, —NR₄R₅ or heteroaryl, wherein R₄ and R₅ each independently is H or hydrocarbyl, or R₄ and R₅ together with the nitrogen atom to which they are attached form a saturated or unsaturated heterocyclic ring optionally containing 1-2 further heteroatoms selected from N, O or S, wherein the additional nitrogen is optionally substituted by alkyl; and

R₂ and R₃ each independently is H or hydrocarbyl;

or an uridine residue of the formula Ib, linked through the oxygen atom linked to the 5′-position:

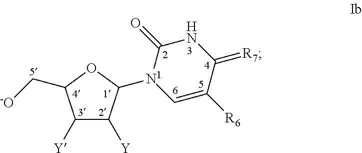

wherein

R₆ is H, halogen, —O-hydrocarbyl, —S-hydrocarbyl, —NR₈R₉, heteroaryl, or hydrocarbyl optionally substituted by one or more groups each independently selected from halogen, —CN, —SCN, —NO₂, —OR₈, —SR₈, —NR₈R₉ or heteroaryl, wherein R₈ and R₉ each independently is H or hydrocarbyl, or R₈ and R₉ together with the nitrogen atom to which they are attached form a saturated or unsaturated heterocyclic ring optionally containing 1-2 further heteroatoms selected from N, O or S, wherein the additional nitrogen is optionally substituted by alkyl; and

R₇ is O or S;

Y and Y′ each independently is H, —OH or —NH₂;

W₁ and W₂ each independently is —O—, —NH— or —C(R₁₀R₁₁)—, wherein R₁₀ and R₁₁ each independently is H or halogen;

Z₁, Z′₁, Z₂, Z′₂ and Z′₃ each independently is O, —O⁻, S, —S⁻ or —BH₃⁻;

Z₃ is —O⁻, —S⁻, —BH₃⁻, or a group of the formula —O—CH₂—OC(O)—R₁₂ or —NH—(CHR₁₃)—C(O)—OR₁₃;

R₁₂ is (C₁-C₄)alkyl;

R₁₃ each independently is (C₁-C₄)alkyl, (C₆-C₁₀)aryl or (C₆-C₁₀)aryl-(C₁-C₄)alkyl;

n is 0 or 1;

m is 2, 3 or 4; and

B⁺ represents a pharmaceutically acceptable cation,

provided that (i) at least one of W₁ and W₂ is not —O—, and at least one of Z₁, Z′₁, Z₂, Z′₂, Z₃ and Z′₃ is S or —S⁻; and (ii) when X is a glucose moiety, Z₃ is —O⁻, —S⁻, or —BH₃⁻; and when X is a group of the formula —O—CH₂—OC(O)—R₁₂ or —NH—(CHR₁₃)—C(O)—OR₁₃, Z₃ is a group of the formula —O—CH₂—OC(O)—R₁₂ or —NH—(CHR₁₃)—C(O)—OR₁₃, respectively.

In another aspect, the present invention provides a pharmaceutical composition comprising a nucleoside 5′-phosphorothioate of the general formula I as defined above, i.e., provided that (i) at least one of W₁ and W₂ is not —O—, and at least one of Z₁, Z′₁, Z₂, Z′₂, Z₃ and Z′₃ is S or —S⁻; and (ii) when X is a glucose moiety, Z₃ is —O⁻, —S⁻, or —BH₃⁻; and when X is a group of the formula —O—CH₂—OC(O)—R₁₂ or —NH—(CHR₁₃)—C(O)—OR₁₃, Z₃ is a group of the formula —O—CH₂—OC(O)—R₁₂ or —NH—(CHR₁₃)—C(O)—OR₁₃, respectively, or a diastereomer or mixture of diastereomers thereof, and a pharmaceutically acceptable carrier or diluent. The compounds and pharmaceutical compositions of the invention are useful, inter alia, in treatment of osteoarthritis/calcium pyrophosphate dihydrate (CPPD) disease.

In yet another aspect, the present invention relates to a method for treatment of osteoarthritis or calcium pyrophosphate dihydrate (CPPD) deposition disease in an individual in need thereof, comprising administering to said individual a therapeutically effective amount of a nucleoside 5′-phosphorothioate of the general formula I as defined above, or a diastereomer or mixture of diastereomers thereof.

### BRIEF DESCRIPTION OF DRAWINGS

FIG. 1 shows Cu⁺ titration of 1 mM Aβ₂₈ solution (pD 7) monitored by ¹H-NMR at 700 MHz.

FIG. 2 shows titration of Aβ₂₈-Cu⁺ complex by various chelators monitored by ¹H-NMR at 700 MHz, pD 7: (a) Aβ₂₈; (b) Aβ₂₈-Cu⁺ 1:1 complex; (c) Aβ₂₈-Cu⁺ complex titrated by 6 eq of clioquinol; (d) Aβ₂₈-Cu⁺ complex titrated by 6 eq of triphosphate; (e) Aβ₂₈-Cu⁺ complex titrated by 6 eq of thiophosphate; (f) Aβ₂₈-Cu⁺ complex titrated by 6 eq of GDP-β-S; (g) Aβ₂₈-Cu⁺ complex titrated by 5 eq of ADP-β-S; and (h) Aβ₂₈-Cu⁺ complex titrated by 3.2 eq of GTP-γ-S.

FIGS. 3A-3B show titration of 9 mM ADP-β-S, pD 7.4, by Cu⁺ monitored by ¹H-NMR at 600 MHz (3A); and ³¹P-NMR at 243 MHz (3B).

FIGS. 4A-4B show detection of free thiophosphate by UV-Vis spectra using Ellmans' reagent (DTNB) in thiophosphate (4A); and GDP-β-S (4B). Abs.—absorption.

FIGS. 5A-5C show disaggregation of Aβ₄₀-M2⁺ by various chelators as measured by DLS: chelator-dependent changes in average d_{H} of Aβ₄₀-Cu²⁺ aggregate (5A); EDTA-relative re-solubilization efficacy of chelators of Aβ₄₀-Zn²⁺ aggregates (5B); and EDTA-relative re-solubilization efficacy of chelators of Aβ₄₀-Cu²⁺ aggregates (5C).

FIGS. 6A-6D show TEM images of 25 μM nine-day-old aggregates: Aβ₄₀-Cu²⁺ aggregate at pH 6.6 (6A); upon addition of APCPP-γ-S (150 μM) to Aβ₄₀-Cu²⁺ aggregate at pH 6.6 (6B); Aβ₄₀-Zn²⁺ aggregate at pH 7.4 (6C); upon addition of APCPP-γ-S (150 μM) to Aβ₄₀-Zn²⁺ aggregate at pH 7.4 (6D).

FIG. 7 shows Aβ₄₀-Cu⁺ titration by APCPP-γ-S monitored by ¹H-NMR at 700 MHz: (a) 0.25 mM Aβ₄₀, pD 11; (b) 0.25 mM Aβ₄₀, pD 7.8; (c) 0.25 mM Aβ₄₀-Cu⁺ 1:1, pD 7.8; and (d) Aβ₂₈-Cu⁺ complex titrated by 6 eq of APCPP-γ-S.

FIGS. 8A-8B show disaggregation of Aβ₄₂-M²⁺ by various chelators as measured by turbidity assay at 405 nm: chelator-dependent changes of Aβ₄₂-Zn²⁺ aggregation relative to EDTA (8A); and chelator-dependent changes of Aβ₄₂-Cu²⁺ aggregation relative to EDTA (8B).

FIG. 9 shows FeSO₄ induced toxicity in cultured cortical neurons. Neurons were exposed to FeSO₄ (0.8-6 μM) for 24 h and toxicity assessed by direct microscopic examination and by XTT assay.

FIGS. 10A-10B show the neuroprotective effect of ATP-γ-S and GDP-β-S (0.2-200 μM, t=24 h) as evaluated by MTT production in cortical neurons exposed for 24 h to either FeSO₄ at final concentration of 3 μM (10A); or both FeSO₄ (3 μM) and H₂O₂ (100 μM) (10B). The results shown are the mean±SEM of three independent experiments in quadruplicate.

FIG. 11 shows application of Aβ₄₂ to neuronal cell culture. Primary neurons cells were cultured in 96 wells plate (95×10⁴ per well). After 24 h the cells were treated with various concentrations (5-50 μM) of Aβ₄₂ for 48 h. Cell viability was measured by dyeing the cells with trypan blue and counts of the vital cells. The results shown are the mean±S.D of three independent experiments in triplicate (*P<0.05, **P<0.01 vs. control).

FIG. 12 shows that APCPP-γ-S protects neuronal cell culture subjected to Aβ₄₂. Primary neuron cells were cultured in 96 wells plate (95×10⁴ per well). After 24 h the cells were treated with 50 μM Aβ₄₂ and various concentrations of APCPP-γ-S (0.04-25 μM) for 48 h. Cell viability was measured by dyeing the cells with trypan blue and counts of the vital cells. The results shown are the mean±S.D of three independent experiments in triplicate (*P<0.05, vs. Aβ₄₂ treatment).

FIG. 13 shows the efficacy of ATP and ATP-γ-S as neuroprotectants against Aβ₄₂ toxicity. Primary neurons cells were cultured in 96 wells plate (95×10⁴ per well). After 24 h the cells were treated with 50 μM of Aβ₄₂ and various concentration of ATP or ATP-γ-S (0.04-25 μM) for 48 h. Cell viability was measured by dyeing the cells with trypan blue and counts the vital cells. The results shown are the mean±S.D of three independent experiments in triplicate (*P<0.05, **P<0.01 vs. Aβ₄₂ treatment).

FIG. 14 shows the efficacy of APCPP-γ-S (1 μM) at P2Y₁-R astrocytoma cells vs. natural ligands (ATP and ADP). Calcium response of astrocytoma cells transfected with plasmids encoding human P2Y₁-GFP receptor fusion protein. Ratio of fluorescence values at 340 nm and 380 nm was calculated (R=ΔF340/380). Basal values were subtracted and the peak height for each cell was determined.

FIG. 15 shows that PC12 cell viability after treatment with APCPP-γ-S. PC12 cells were treated with 1-1000 μM APCPP-γ-S, and after 24 h cell viability was measured by the MTT assay, compared to non-treated cells. The results shown are the mean±S.D of three independent experiments in triplicate.

FIG. 16 demonstrates a kinetic profile showing the changes in the percentage of adenosine-5′-tetrathiobisphosphonate in acidic conditions (pD=1.5), as monitored by ³¹P-NMR at 81 MHz, at 300 K.

FIG. 17 demonstrates a kinetic profile showing the changes in the percentage of adenosine-5′-tetrathiobisphosphonate subjected to air-oxidation, as monitored by ³¹P-NMR at 81 MHz, at 300 K.

FIG. 18 shows ³¹P-NMR spectra of di-adenosine-5′,5″-tetrathiobisphosphonate at pD=1.5.

FIGS. 19A-19B show titration of 3 mM di-adenosine-5′,5″-tetrathiobis phosphonate in D₂O at pD=7.38 with Zn²⁺. ³¹P-NMR spectrum was measured at 160 MHz, 300K (19A); and ¹H-NMR spectrum was measured at 400 MHz, 300K (19B).

FIGS. 20A-20B show titration of 5 mM adenosine-5′-tetrathiobisphosphonate in D₂O at pD=7.40 with Zn²⁺. ³¹P-NMR spectrum was measured at 160 MHz, 300K; (20A); and ¹H-NMR spectrum was measured at 400 MHz, 300K (20B).

FIGS. 21A-21C show inhibition of pnp-TMP and ATP hydrolysis with NPP1,3 and NTPDase1,2,3,8, respectively, by adenosine-5′-tetrathiobisphosphonate (21A); di-adenosine-5′,5″-tetrathiobisphosphonate (21B); and ADP-β-S (21C).

FIGS. 22A-C show that APPCP-α-S, APPCCl₂P-α-S and APCPP-γ-S (100 μM) inhibit NPP activities. Activity of human NPP1 (hNPP1) and NPP3 (hNPP3) was tested with pNP-TMP (22A, 22C) or ATP (22B) as the substrate at 100 μM. The 100% activity with pNP-TMP alone was 48±4 and 32±2 [nmol p-nitrophenol·min⁻¹·mg protein⁻¹] for NPP1 and NPP3, respectively (22A). The 100% activity with ATP alone was 153±6 and 110±5 [nmol nucleotide·min⁻¹·mg protein⁻¹] for NPP1 and NPP3, respectively (22B). Data presented are the mean±SD of 3 experiments carried out in triplicates. 22C). The analogues inhibit NPP activity at the surface of HTB85 cells. The 100% NPP activity was set with the substrate alone and was 1.3±0.04 [nmol p-nitrophenol·min⁻¹·well]. Data presented are the means±SD of results from 3 experiments carried out in triplicates.

FIGS. 23A-B show K_{i,app} determination using Dixon (23A) and Cornish-Bowden (23B) plot, of human NPP1 by APPCCl₂P-α-S (isomer A). pNP-TMP concentrations were 25, 50 and 100 μM, and the inhibitor concentrations were 0, 25, 50 and 100 μM.

FIG. 24 shows the activity of APPCP-α-S (isomers A and B) at the P2Y₁₁R. Data were obtained by determining the ligand-induced change in [Ca²⁺]ᵢ in 1321N1 cells stably expressing the human GFP-P2Y₁₁R. Cells were pre-incubated with 2 μM fura-2 AM for 30 min and change in fluorescence (ΔF340 nm/F380 nm) was detected.

FIG. 25 shows that APPCCl₂P-α-S (isomer A) inhibits ATP hydrolysis in the presence of human chondrocyte cells. Values represent mean±S.D. of three experiments (P<0.05).

FIG. 26 shows the ability of APPCCl₂P-α-S (isomer A) to inhibit the hydrolysis of NTMP in MVs. At each time point the reaction was read at 405 nm.

FIG. 27 shows the ability of APPCCl₂P-α-S (isomer A) to inhibit the hydrolysis of NTMP in human chondrocyte cells. At each time point the reaction was read at 405 nm.

FIG. 28 shows the ability of APPCCl₂P-α-S (isomer A) to inhibit the hydrolysis of NTMP in cartilage pieces. At each time point 200 μl were removed from each well and the reaction was read at 405 nm.

FIG. 29 shows standard curve of pyrophosphate as measured by pyrophosphate assay kit.

FIG. 30 shows FTIR spectra of matrix vesicles (MV) mineralization in the absence and presence of ATP and APPCCl₂P-α-S (isomer A). FTIR spectrum of MVs mineralized in the absence of any substrate is indicated as control. Absorbance at 920 and 1125 cm⁻¹ are characteristic of CPPD.

### DETAILED DESCRIPTION OF THE INVENTION

The present invention provides, in one aspect, a compound of the general formula I:

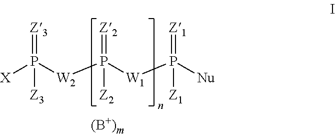

or a diastereomer or mixture of diastereomers thereof,

wherein

X is —O⁻, Nu′, a glucose moiety linked through the oxygen atom linked to its 1- or 6-position, or a group of the formula —O—CH₂—OC(O)—R₁₂ or —NH—(CHR₁₃)—C(O)—OR₁₃;

Nu and Nu′ each independently is an adenosine residue of the formula Ia, linked through the oxygen atom linked to the 5′-position:

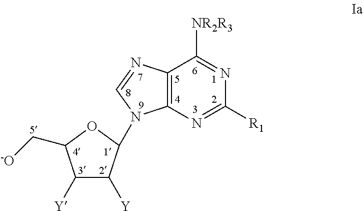

wherein

R₁ is H, halogen, —O-hydrocarbyl, —S-hydrocarbyl, —NR₄R₅, heteroaryl, or hydrocarbyl optionally substituted by one or more groups each independently selected from halogen, —CN, —SCN, —NO₂, —OR₄, —SR₄, —NR₄R₅ or heteroaryl, wherein R₄ and R₅ each independently is H or hydrocarbyl, or R₄ and R₅ together with the nitrogen atom to which they are attached form a saturated or unsaturated heterocyclic ring optionally containing 1-2 further heteroatoms selected from N, O or S, wherein the additional nitrogen is optionally substituted by alkyl; and

R₂ and R₃ each independently is H or hydrocarbyl;

or an uridine residue of the formula Ib, linked through the oxygen atom linked to the 5′-position:

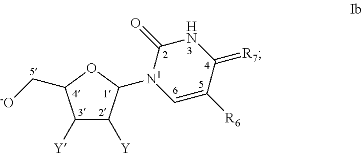

wherein

R₆ is H, halogen, —O-hydrocarbyl, —S-hydrocarbyl, —NR₈R₉, heteroaryl, or hydrocarbyl optionally substituted by one or more groups each independently selected from halogen, —CN, —SCN, —NO₂, —OR₈, —SR₈, —NR₈R₉ or heteroaryl, wherein R₈ and R₉ each independently is H or hydrocarbyl, or R₈ and R₉ together with the nitrogen atom to which they are attached form a saturated or unsaturated heterocyclic ring optionally containing 1-2 further heteroatoms selected from N, O or S, wherein the additional nitrogen is optionally substituted by alkyl; and

R₇ is O or S;

Y and Y′ each independently is H, —OH or —NH₂;

W₁ and W₂ each independently is —O—, —NH— or —C(R₁₀R₁₁)—, wherein R₁₀ and R₁₁ each independently is H or halogen;

Z₁, Z′₁, Z₂, Z′₂ and Z′₃ each independently is O, —O⁻, S, —S⁻ or —BH₃⁻;

Z₃ is —O⁻, —S⁻, —BH₃⁻, or a group of the formula —O—CH₂—OC(O)—R₁₂ or —NH—(CHR₁₃)—C(O)—OR₁₃;

R₁₂ is (C₁-C₄)alkyl;

R₁₃ each independently is (C₁-C₄)alkyl, (C₆-C₁₀)aryl or (C₆-C₁₀)aryl-(C₁-C₄)alkyl;

n is 0 or 1;

m is 2, 3 or 4; and

B⁺ represents a pharmaceutically acceptable cation,

provided that (i) at least one of W₁ and W₂ is not —O—, and at least one of Z₁, Z′₁, Z₂, Z′₂, Z₃ and Z′₃ is S or —S⁻; and (ii) when X is a glucose moiety, Z₃ is —O⁻, —S⁻, or —BH₃⁻; and when X is a group of the formula —O—CH₂—OC(O)—R₁₂ or —NH—(CHR₁₃)—C(O)—OR₁₃, Z₃ is a group of the formula —O—CH₂—OC(O)—R₁₂ or —NH—(CHR₁₃)—C(O)—OR₁₃, respectively.

The compound of the present invention may be an adenosine- or uridine-5′-di- or tri-phosphorothioate derivative, as well as a dinucleoside 5′-di- or tri-phosphorothioate derivative in which each one of the two nucleosides may independently be an adenosine derivative or an uridine derivative, but preferably both nucleosides are identical. In a further configuration, the compound of the present invention is a mono-nucleoside 5′-di- or tri-phosphorothioate derivative in the form of a prodrug, wherein (i) one of the non-bridging oxygen atoms at position β of the diphosphorothioate, or at position γ of the triphosphorothioate, is replaced by a glucose moiety linked through the oxygen atom linked to its 1- or 6-position; or (ii) two of the non-bridging oxygen atoms at position β of the diphosphorothioate, or at position γ of the triphosphorothioate, are each replaced by a group of the formula —O—CH₂—OC(O)—R₁₂ or —NH—(CHR₁₃)—C(O)—OR₁₃, wherein R₁₂ is (C₁-C₄)alkyl, and R₁₃ each independently is (C₁-C₄)alkyl, (C₆-C₁₀)aryl or (C₆-C₁₀)aryl-(C₁-C₄)alkyl. The common feature unifying all these compounds is the fact that at least one of the bridging oxygen atoms of the phosphorothioate, i.e., either or both the α,β- and β,γ-bridging-oxygen atoms, is replaced by a group selected from —NH— or —C(R₁₀R₁₁)—, wherein R₁₀ and R₁₁ each independently is H or halogen, preferably by CH₂, CCl₂ or CF₂, and at least one, i.e., 1, 2, 3, 4, 5 or 6, of the non-bridging atoms or negatively-charged atoms of the phosphorothioate is either a sulfur atom (S) or a sulfur ion (S⁻).

As used herein, the term “halogen” includes fluoro, chloro, bromo, and iodo, and is preferably fluoro or chloro.

The term “hydrocarbyl” in any of the definitions of the different radicals R₁ to R₉ refers to a radical containing only carbon and hydrogen atoms that may be saturated or unsaturated, linear or branched, cyclic or acyclic, or aromatic, and includes (C₁-C₈)alkyl, (C₂-C₈)alkenyl, (C₂-C₈)alkynyl, (C₃-C₁₀)cycloalkyl, (C₃-C₁₀)cycloalkenyl, (C₆-C₁₄)aryl, (C₁-C₈)alkyl(C₆-C₁₄)aryl, and (C₆-C₄)aryl(C₁-C₈)alkyl.

The term “C₁-C₈)alkyl” typically means a linear or branched hydrocarbon radical having 1-8 carbon atoms and includes, e.g., methyl, ethyl, n-propyl, isopropyl, n-butyl, sec-butyl, isobutyl, tert-butyl, n-pentyl, 2,2-dimethylpropyl, n-hexyl, n-heptyl, n-octyl, and the like. Preferred are (C₁-C₆)alkyl groups, more preferably (C₁-C₄)alkyl groups, most preferably methyl and ethyl. The terms “(C₂-C₈)alkenyl” and “C₂-C₈)alkynyl” typically mean straight and branched hydrocarbon radicals having 2-8 carbon atoms and 1 double or triple bond, respectively, and include ethenyl, 3-buten-1-yl, 2-ethenylbutyl, 3-octen-1-yl, and the like, and propynyl, 2-butyn-1-yl, 3-pentyn-1-yl, and the like. (C₂-C₆)alkenyl and (C₂-C₆)alkynyl radicals are preferred.

The term “(C₃-C₁₀)cycloalkyl” as used herein means a mono- or bicyclic saturated hydrocarbyl group having 3-10 carbon atoms such as cyclopropyl, cyclobutyl, cyclopentyl, cyclohexyl, cycloheptyl, cyclooctyl, adamantyl, bicyclo[3.2.1]octyl, bicyclo[2.2.1]heptyl, and the like, which may be substituted, e.g., with one or more groups each independently selected from halogen, e.g., F, Cl or Br, —OH, —NO₂, —CN, —SCN, (C₁-C₈)alkyl, —O—(C₁-C₈)alkyl, —S—(C₁-C₈)alkyl, —NH₂, —NH—(C₁-C₈)alkyl, or —N—((C₁-C₈)alkyl)₂.

The term “(C₆-C₁₄)aryl” denotes an aromatic carbocyclic aromatic group having 6-14 carbon atoms consisting of a single ring or multiple rings either condensed or linked by a covalent bonf such as, but not limited to, phenyl, naphthyl, phenanthryl and biphenyl. Preferred are (C₆-C₁₀)aryl, more preferably phenyl. The aryl radical may optionally be substituted by one or more groups each independently selected from halogen, e.g., F, Cl or Br, —OH, —NO₂, —CN, —SCN, (C₁-C₈)alkyl, —O—(C₁-C₈)alkyl, —S—(C₁-C₈)alkyl, —NH₂, —NH—(C₁-C₈)alkyl, or —N—((C₁-C₈)alkyl)₂. The term “ar(C₁-C₈)alkyl” denotes an arylalkyl radical such as benzyl and phenetyl.

The term “heteroaryl” refers to a radical derived from a mono- or poly-cyclic heteroaromatic ring containing one to three, preferably 1 or 2, heteroatoms selected from N, O or S. When the heteroaryl is a monocyclic ring, it is preferably a radical of a 5-6-membered ring such as, but not limited to, pyrrolyl, furyl, thienyl, thiazinyl, pyrazolyl, pyrazinyl, imidazolyl, oxazolyl, isoxazolyl, thiazolyl, isothiazolyl, pyridyl, pyrimidinyl, 1,2,3-triazinyl, 1,3,4-triazinyl, and 1,3,5-triazinyl. Polycyclic heteroaryl radicals are preferably composed of two rings such as, but not limited to, benzofuryl, isobenzofuryl, benzothienyl, indolyl, quinolinyl, isoquinolinyl, imidazo[1,2-a]pyridyl, benzimidazolyl, benzthiazolyl, benzoxazolyl, pyrido[1,2-a]pyrimidinyl and 1,3-benzodioxinyl. The heteroaryl ring may be substituted. It is to be understood that when a polycyclic heteroaromatic ring is substituted, the substitution may be in the heteroring or in the carbocyclic ring.

The term “heterocyclic ring” denotes a mono- or poly-cyclic non-aromatic ring of 4-12 atoms containing at least one carbon atom and one to three heteroatoms selected from sulfur, oxygen or nitrogen, which may be saturated or unsaturated, i.e., containing at least one unsaturated bond. Preferred are 5- or 6-membered heterocyclic rings. Non-limiting examples of radicals —NR₄R₅ and —NR₈R₉ include amino, dimethylamino, diethylamino, ethylmethylamino, phenylmethyl-amino, pyrrolidino, piperidino, tetrahydropyridino, piperazino, ethylpiperazino, hydroxyethyl piperazino, morpholino, thiomorpholino, thiazolino, and the like.

In certain embodiments, the compound of the present invention is a mono- or dinucleoside 5′-phosphorothioate of the general formula I as defined above, or a diastereomer or mixture of diastereomers thereof, wherein Nu and Nu′, if present, each independently is an adenosine residue of the formula Ia, wherein R₁ is H, halogen, —O-hydrocarbyl, —S-hydrocarbyl, —NR₄R₅, heteroaryl, or hydrocarbyl; R₄ and R₅ each independently is H or hydrocarbyl, or R₄ and R₅ together with the nitrogen atom to which they are attached form a 5- or 6-membered saturated or unsaturated heterocyclic ring optionally containing 1-2 further heteroatoms selected from N, O or S; said hydrocarbyl each independently is (C₁-C₈)alkyl, (C₂-C₈)alkenyl, (C₂-C₈)alkynyl, or (C₆-C₁₄)aryl; and said heteroaryl is a 5-6-membered monocyclic heteroaromatic ring containing 1-2 heteroatoms selected from N, O or S. In particular such embodiments, R₁ is H, —O-hydrocarbyl, —S-hydrocarbyl, —NR₄R₅, or hydrocarbyl; R₄ and R₅ each independently is H or hydrocarbyl; and said hydrocarbyl each independently is (C₁-C₄)alkyl, preferably methyl or ethyl, (C₂-C₄)alkenyl, (C₂-C₄)alkynyl, or (C₆-C₁₀)aryl, preferably phenyl. More particular such embodiments are those, wherein R₁ is H, —O-hydrocarbyl, —S-hydrocarbyl, —NR₄R₅, or hydrocarbyl; R₄ and R₅ each independently is H or hydrocarbyl; and said hydrocarbyl each independently is methyl or ethyl.

In certain embodiments, the compound of the present invention is a mono- or dinucleoside 5′-phosphorothioate of the general formula I as defined above, or a diastereomer or mixture of diastereomers thereof, wherein Nu and Nu′, if present, each independently is an adenosine residue of the formula Ia, wherein R₂ and R₃ each independently is H or hydrocarbyl; and said hydrocarbyl is (C₁-C₄)alkyl, preferably methyl or ethyl, (C₂-C₄)alkenyl, (C₂-C₄)alkynyl, or (C₆-C₁₀)aryl, preferably phenyl.

In certain embodiments, the compound of the present invention is a mono- or dinucleoside 5′-phosphorothioate of the general formula I as defined above, or a diastereomer or mixture of diastereomers thereof, wherein Nu and Nu′, if present, each is an adenosine residue of the formula Ia, wherein R₁, R₂ and R₃ are H, i.e., a purine nucleoside comprising a molecule of adenine linked to a moiety of either ribofuranose or a ribofuranose derivative via a β-N₉-glycosidic bond.

In certain embodiments, the compound of the present invention is a mono- or dinucleoside 5′-phosphorothioate of the general formula I as defined above, or a diastereomer or mixture of diastereomers thereof, wherein Nu and Nu′, if present, each independently is an uridine residue of the formula Ib, wherein R₆ is H, halogen, —O-hydrocarbyl, —S-hydrocarbyl, —NR₈R₉, heteroaryl, or hydrocarbyl; R₈ and R₉ each independently is H or hydrocarbyl, or R₈ and R₉ together with the nitrogen atom to which they are attached form a 5- or 6-membered saturated or unsaturated heterocyclic ring optionally containing 1-2 further heteroatoms selected from N, O or S; said hydrocarbyl each independently is (C₁-C₈)alkyl, (C₂-C₈)alkenyl, (C₂-C₈)alkynyl, or (C₆-C₁₄)aryl; and said heteroaryl is a 5-6-membered monocyclic heteroaromatic ring containing 1-2 heteroatoms selected from N, O or S. In particular such embodiments, R₆ is H, —O-hydrocarbyl, —S-hydrocarbyl, —NR₈R₉, or hydrocarbyl; R₈ and R₉ each independently is H or hydrocarbyl; and said hydrocarbyl each independently is (C₁-C₄)alkyl, preferably methyl or ethyl, (C₂-C₄)alkenyl, (C₂-C₄)alkynyl, or (C₆-C₁₀)aryl, preferably phenyl. More particular such embodiments are those, wherein R₆ is H, —O-hydrocarbyl, —S-hydrocarbyl, —NR₈R₉, or hydrocarbyl; R₈ and R₉ each independently is H or hydrocarbyl; and said hydrocarbyl each independently is methyl or ethyl.

In certain embodiments, the compound of the present invention is a mono- or dinucleoside 5′-phosphorothioate of the general formula I as defined above, or a diastereomer or mixture of diastereomers thereof, wherein Nu and Nu′, if present, each independently is an uridine residue of the formula Ib, wherein R₇ is O.

In certain embodiments, the compound of the present invention is a mono- or dinucleoside 5′-phosphorothioate of the general formula I as defined above, or a diastereomer or mixture of diastereomers thereof, wherein Nu and Nu′, if present, each is an uridine residue of the formula Ib, wherein R₆ is H; and R₇ is O, i.e., a pyrimidine nucleoside comprising a molecule of uracil linked to a moiety of either ribofuranose or a ribofuranose derivative via a β-N₁-glycosidic bond.

In certain embodiments, the compound of the present invention is a mono- or dinucleoside 5′-phosphorothioate of the general formula I as defined above, or a diastereomer or mixture of diastereomers thereof, wherein Y′ each independently is —OH; and Y each independently is H or —OH.

In certain embodiments, the compound of the present invention is a mono- or dinucleoside 5′-phosphorothioate of the general formula I as defined above, or a diastereomer or mixture of diastereomers thereof, wherein W₁ and W₂ each independently is —O— or —C(R₁₀R₁₁)—, wherein R₁₀ and R₁₁ each independently is H, Cl or F, preferably H or Cl.

In certain embodiments, the compound of the present invention is a mono- or dinucleoside 5′-phosphorothioate of the general formula I as defined above, or a diastereomer or mixture of diastereomers thereof, wherein n is 0, i.e., an adenosine- or uridine-5′-diphosphorothioate derivative, as well as a dinucleoside 5′-di-phosphorothioate derivative in which each one of the two nucleosides may independently be either an adenosine derivative or an uridine derivative as defined in any one of the embodiments above, but preferably both nucleosides are identical.

In other embodiments, the compound of the present invention is a mono- or dinucleoside 5′-phosphorothioate of the general formula I as defined above, or a diastereomer or mixture of diastereomers thereof, wherein n is 1, i.e., an adenosine- or uridine-5′-triphosphorothioate derivative, as well as a dinucleoside 5′-triphosphorothioate derivative in which each one of the two nucleosides may independently be either an adenosine derivative or an uridine derivative as defined in any one of the embodiments above, but preferably both nucleosides are identical.

In certain particular embodiments, the compound of the present invention is a mononucleoside 5′-phosphorothioate of the general formula I as defined in any one of the embodiments above, wherein X is —O⁻, i.e., a mono-nucleoside 5′-di- or tri-phosphorothioate. In other particular embodiments, the compound of the invention is a dinucleoside 5′-phosphorothioate of the general formula I as defined in any one of the embodiments above, wherein X is Nu′, i.e., a dinucleoside 5′-di- or tri-phosphorothioate. In further particular embodiments, the compound of the invention is a mononucleoside 5′-phosphorothioate of the general formula I as defined in any one of the embodiments above, wherein X is a glucose moiety linked through the oxygen atom linked to its 1- or 6-position, i.e., a mononucleoside 5′-di- or tri-phosphorothioate in one particular form of a prodrug. According to the invention, at least one of the bridging oxygen atoms of the phosphorothioate in all these compounds is replaced by a group selected from —NH— or —C(R₁₀R₁₁)— as defined above, and one or more of the non-bridging atoms or negatively-charged atoms of the phosphorothioate is either a sulfur atom (S) or a sulfur ion (S⁻).

In certain more particular such embodiments, the compound of the present invention is a mono- or dinucleoside 5′-phosphorothioate of the general formula I as defined hereinabove, i.e., when X is —O⁻, Nu′, or a glucose moiety, wherein n is 0; W₂ is —C(R₁₀R₁₁)—, preferably wherein R₁₀ and R₁₁ each is H, Cl or F; and 1, 2, 3 or 4 of Z₁, Z′₁, Z₃ and Z′₃ is S or —S⁻, i.e., (i) one of Z₁ and Z′₁ is —S⁻ or S, and another of Z₁ and Z′₁, Z₃ and Z′₃ each independently is O or —O⁻; or one of Z₃ and Z′₃ is —S⁻ or S, and Z₁, Z′₁, and another of Z₃ and Z′₃, each independently is O or —O⁻; (ii) one of Z₁ and Z′₁, and one of Z₃ and Z′₃, each independently is —S⁻ or S, and the other of Z₁, Z′₁, Z₃ and Z′₃ each independently is O or —O⁻; Z₁ and Z′₁ each independently is —S⁻ or S, and Z₃ and Z′₃ each independently is O or —O⁻; or Z₃ and Z′₃ each independently is —S⁻ or S, and Z₁ and Z′₁ each independently is O or —O⁻; (iii) Z₁, Z′₁, and one of Z₃ and Z′₃, each independently is —S⁻ or S, and another of Z₃ and Z′₃ is O or —O⁻; or Z₃, Z′₃, and one of Z₁ and Z′₁, each independently is —S⁻ or S, and another of Z₁ and Z′₁ is O or —O⁻; or (iv) Z₁, Z′₁, Z₃ and Z′₃ each independently is —S⁻ or S.

Particular such compounds are those wherein Y and Y′ are —OH; n is 0; W₂ is —CH₂—, —CCl₂— or —CF₂—; and Nu and Nu′, if present, each is (i) an adenosine residue of the formula Ia, wherein R₁, R₂ and R₃ are H; or (ii) an uridine residue of the formula Ib, wherein R₆ is H; and R₇ is O, Specific such compounds exemplified herein are those wherein (i) X is —O⁻; Nu is an adenosine residue of the formula Ia, wherein R₁, R₂ and R₃ are H, or an uridine residue of the formula Ib, wherein R₆ is H, and R₇ is O; Y and Y′ are —OH; n is 0; W₂ is —CH₂—; and Z₁, Z′₁, Z₃ and Z′₃ are —S⁻ or S (adenosine-5′-tetrathiobisphosphonate and uridine-5′-tetrathiobisphosphonate, herein also identified “APCP-α,α′,β,β′-tetra-S” and “UPCP-α,α′,β,β′-tetra-S”, respectively); or (ii) X is Nu′; Nu and Nu′ each is an adenosine residue of the formula Ia, wherein R₁, R₂ and R₃ are H, or an uridine residue of the formula Ib, wherein R₆ is H, and R₇ is O; Y and Y′ are —OH; n is 0; W₂ is —CH₂—; and Z₁, Z′₁, Z₃ and Z′₃ are —S⁻ or S (di-adenosine-5′,5″-tetrathiobisphosphonate and di-uridine-5′,5″-tetrathiobisphosphonate, herein also identified “APCPA-α,α′,β,β′-tetra-S” and “UPCPU-α,α′,β,β′-tetra-S”, respectively).

In other more particular such embodiments, the compound of the present invention is a mono- or dinucleoside 5′-phosphorothioate of the general formula I as defined hereinabove, i.e., when X is —O⁻, Nu′, or a glucose moiety, wherein n is 1; either one of W₁ and W₂ is —O— and another of W₁ and W₂ is —C(R₁₀R₁₁)—, or both W₁ and W₂ each independently is —C(R₁₀R₁₁)—, preferably wherein R₁₀ and R₁₁ each is H, Cl or F; and 1, 2, 3, 4, 5 or 6 of Z₁, Z′₁, Z₂, Z′₂, Z₃ and Z′₃ is S or —S⁻, i.e., (i) one of Z₁ and Z′₁ is —S⁻ or S, and another of Z₁ and Z′₁, Z₂, Z′₂, Z₃ and Z′₃ each independently is O or —O⁻; one of Z₂ and Z′₂ is —S⁻ or S, and Z₁, Z′₁, another of Z₂ and Z′₂, Z₃ and Z′₃ each independently is O or —O⁻; or one of Z₃ and Z′₃ is —S⁻ or S, and Z₁, Z′₁, Z₂, Z′₂, and another of Z₃ and Z′₃, each independently is O or —O⁻; (ii) one of Z₁ and Z′₁, and one of Z₂ and Z′₂, each independently is —S⁻ or S, and the other of Z₁, Z′₁, Z₂, Z′₂, and Z₃ and Z′₃, each independently is O or —O⁻; one of Z₁ and Z′₁, and one of Z₃ and Z′₃, each independently is —S⁻ or S, and the other of Z₁, Z′₁, Z₃, Z′₃, and Z₂ and Z′₂, each independently is O or —O⁻; one of Z₂ and Z′₂, and one of Z₃ and Z′₃, each independently is —S⁻ or S, and Z₁, Z′₁, and the other of Z₂, Z′₂, Z₃, Z′₃, each independently is O or —O⁻; Z₁ and Z′₁ each independently is —S⁻ or S, and Z₂, Z′₂, Z₃ and Z′₃ each independently is O or —O⁻; Z₂ and Z′₂ each independently is —S⁻ or S, and Z₁, Z′₁, Z₃ and Z′₃ are O or —O⁻; or Z₃ and Z′₃ each independently is —S⁻ or S, and Z₁, Z′₁, Z₂ and Z′₂ are O or —O⁻; (iii) one of Z₁ and Z′₁, one of Z₂ and Z′₂, and one of Z₃ and Z′₃, each independently is —S⁻ or S, and the other of Z₁, Z′₁, Z₂, Z′₂, Z₃ and Z′₃ each independently is O or —O⁻; Z₁ and Z′₁, and one of Z₂ and Z′₂, each independently is —S⁻ or S, and another of Z₂ and Z′₂, Z₃ and Z′₃ each independently is O or —O⁻; Z₁ and Z′₁, and one of Z₃ and Z′₃, each independently is —S⁻ or S, and Z₂, Z′₂, and another of Z₃ and Z′₃ each independently is O or —O⁻; Z₂ and Z′₂, and one of Z₁ and Z′₁, each independently is —S⁻ or S, and another of Z₁ and Z′₁, Z₃ and Z′₃ each independently is O or —O⁻; Z₂ and Z′₂, and one of Z₃ and Z′₃, each independently is —S⁻ or S, and Z₁, Z′₁, and another of Z₃ and Z′₃, each independently is O or —O⁻; Z₃ and Z′₃, and one of Z₁ and Z′₁, each independently is —S⁻ or S, and another of Z₁ and Z′₁, Z₂ and Z′₂ each independently is O or —O⁻; or Z₃ and Z′₃, and one of Z₂ and Z′₂, each independently is —S⁻ or S, and Z₁, Z′₁, and another of Z₂ and Z′₂, each independently is O or —O⁻; (iv) Z₁, Z′₁, one of Z₂ and Z′₂, and one of Z₃ and Z′₃, each independently is —S⁻ or S, and the other of Z₂, Z′₂, Z₃ and Z′₃ each independently is O or —O⁻; Z₂, Z′₂, one of Z₁ and Z′₁, and one of Z₃ and Z′₃, each independently is —S⁻ or S, and the other of Z₁, Z′₁, Z₃ and Z′₃ each independently is O or —O⁻; Z₃, Z′₃, one of Z₁ and Z′₁, and one of Z₂ and Z′₂, each independently is —S⁻ or S, and the other of Z₁, Z′₁, Z₂ and Z′₂ each independently is O or —O⁻; Z₁, Z′₁, Z₂ and Z′₂ each independently is —S⁻ or S, and Z₃ and Z′₃ each independently is O or —O⁻; Z₁, Z′₁, Z₃ and Z′₃ each independently is —S⁻ or S, and Z₂ and Z′₂ each independently is O or —O⁻; or Z₂, Z′₂, Z₃ and Z′₃ each independently is —S⁻ or S, and Z₁ and Z′₁ each independently is O or —O⁻; (v) Z₁, Z′₁, Z₂, Z′₂, and one of Z₃ and Z′₃, each independently is —S⁻ or S, and another of Z₃ and Z′₃ is O or —O⁻; Z₁, Z′₁, Z₃, Z′₃, and one of Z₂ and Z′₂, each independently is —S⁻ or S, and another of Z₂ and Z′₂ is O or —O⁻; or Z₂, Z′₂, Z₃, Z′₃, and one of Z₁ and Z′₁, each independently is —S⁻ or S, and another of Z₁ and Z′₁ is O or —O⁻; or (vi) Z₁, Z′₁, Z₂, Z′₂, Z₃ and Z′₃ each independently is —S⁻ or S.

Particular such compounds are those wherein X is —O⁻, Nu′, or a glucose moiety; Y and Y′ are —OH; n is 1; either one of W₁ and W₂ is —O— and another of W₁ and W₂ is —CH₂—, —CCl₂— or —CF₂—, or both W₁ and W₂ are —CH₂—, —CCl₂— or —CF₂—; and Nu and Nu′, if present, each is (i) an adenosine residue of the formula Ia, wherein R₁, R₂ and R₃ are H; or (ii) an uridine residue of the formula Ib, wherein R₆ is H, and R₇ is O. Specific such compounds exemplified herein are those wherein (i) X is —O⁻; Nu is an adenosine residue of the formula Ia, wherein R₁, R₂ and R₃ are H; Y and Y′ are —OH; n is 1; W₁ is —CH₂—; W₂ is —O⁻; and one of Z₃ and Z′₃ is —S⁻ or S, and Z₁, Z′₁, Z₂, Z′₂, and another of Z₃ and Z′₃, are O or —O⁻ (adenosine 5′-[Pγ-thio]-αβ-methylene triphosphate, herein also identified “APCPP-γ-S”); (ii) X is —O⁻; Nu is an adenosine residue of the formula Ia, wherein R₁, R₂ and R₃ are H; Y and Y′ are —OH; n is 1; W₁ is —O⁻; W₂ is —CH₂—; and one of Z₁ and Z′₁ is —S⁻ or S, and another of Z₁ and Z′₁, Z₂, Z′₂, Z₃ and Z′₃ are O or —O⁻ (adenosine 5′-[Pc-thio]-β,γ-methylene triphosphate, herein also identified “APPCP-α-S”); or (iii) X is —O⁻; Nu is an adenosine residue of the formula Ia, wherein R₁, R₂ and R₃ are H; Y and Y′ are —OH; n is 1; W₁ is —O⁻; W₂ is —CCl₂—; and one of Z₁ and Z′₁ is —S⁻ or S, and another of Z₁ and Z′₁, Z₂, Z′₂, Z₃ and Z′₃ are O or —O⁻ (adenosine 5′-[Pα-thio]-β,γ-(dichloromethylene)triphosphate, herein also identified “APPCCl₂P-α-S”), or a prodrug of APCPP-γ-S wherein X is a glucose moiety linked through the oxygen atom linked to its 1-position; Nu is an adenosine residue of the formula Ia, wherein R₁, R₂ and R₃ are H; Y and Y′ are —OH; n is 1; W₁ is —CH₂—; W₂ is —O⁻; and one of Z₃ and Z′₃ is —S⁻ or S, and Z₁, Z′₁, Z₂, Z′₂, and another of Z₃ and Z′₃, are O or —O⁻ (D-glucosyl-1-adenosine 5′-[γ-thio]-α,β-methylene triphosphate, herein also identified “I-D-glucosyl-Pγ-APCPP-γ-S”). In a specific embodiment, the compound of the present invention is the diastereoisomer A of adenosine 5′-[Pα-thio]-β,γ-(dichloromethylene)triphosphate, (APPCCl₂P-α-S (isomer A)), characterized by being the isomer with a retention time (Rt) of 20.3 min when separated from a mixture of diastereoisomers using a semi-preparative reverse-phase Gemini 5u column (C-18 110A, 250×10 mm, 5 μm; Phenomenex, Torrance, Calif.), and gradient elution from 96.5:3.5 to 95.5:4.5 [100 mM TEAA, pH 7:CH₃CN] over 31 min at a flow rate of 4.5 ml/min. The purity of APPCCl₂P-α-S, isomer A, was evaluated on an analytical reverse-phase HPLC column system [Gemini 5u C-18 110A, 150×3.60 mm, 5 μm (Phenomenex)] in two-solvent systems with either solvent system I or II. Solvent system I consisted of 100 mM TEAA (pH 7) and CH₃CN. Solvent system II consisted of 46 mM PBS (pH 7.4) and CH₃CN. Isomer A of had a retention time of 9.5 min (99% purity) using solvent system I with a TEAA/CH₃CN isocratic elution 96:4 over 15 min at a flow rate of 1 ml/min; and 3.35 min (99% purity) using solvent system II with a PBS/CH₃CN isocratic elution 98:2 over 8 min at a flow rate of 1 ml/min.

In certain particular embodiments, the compound of the present invention is a mononucleoside 5′-phosphorothioate of the general formula I as defined in any one of the embodiments above, wherein X is a group of the formula —O—CH₂—OC(O)—R₁₂ or —NH—(CHR₁₃)—C(O)—OR₁₃; R₁₂ is (C₁-C₄)alkyl; and R₁₃ each independently is (C₁-C₄)alkyl, (C₆-C₁₀)aryl or (C₆-C₁₀)aryl-(C₁-C₄)alkyl, i.e., a mono- or dinucleoside 5′-phosphorothioate in one of two additional forms of a prodrug. According to the invention, at least one of the bridging oxygen atoms of the phosphorothioate in all these compounds is replaced by a group selected from —NH— or —C(R₁₀R₁₁)— as defined above, and one or more of the non-bridging atoms or negatively-charged atoms of the phosphorothioate is either a sulfur atom (S) or a sulfur ion (S⁻).

In certain more particular such embodiments, the compound of the present invention is a mononucleoside 5′-phosphorothioate of the general formula I as defined hereinabove, i.e., when X is a group of the formula —O—CH₂—OC(O)—R₁₂ or —NH—(CHR₁₃)—C(O)—OR₁₃, wherein n is 0, W₂ is —C(R₁₀R₁₁)—, preferably wherein R₁₀ and R₁₁ each is H, Cl or F, and 1, 2 or 3 of Z₁, Z′₁ and Z′₃ is S or —S⁻, i.e., (i) one of Z₁ and Z′₁ is —S⁻ or S, and another of Z₁ and Z′₁, and Z′₃ each independently is O or —O⁻; or Z′₃ is —S⁻ or S, and Z₁ and Z′₁ each independently is O or —O⁻; (ii) one of Z₁ and Z′₁, and Z′₃, each independently is —S⁻ or S, and the other of Z₁ and Z′₁ is O or —O⁻; or Z₁ and Z′₁ each independently is —S⁻ or S, and Z′₃ is O or —O⁻; and (iii) Z₁, Z′₁ and Z′₃ each independently is —S⁻ or S.

In other more particular such embodiments, the compound of the present invention is a mononucleoside 5′-phosphorothioate of the general formula I as defined hereinabove, i.e., when X is group of the formula —O—CH₂—OC(O)—R₁₂ or —NH—(CHR₁₃)—C(O)—OR₁₃, wherein n is 1, either one of W₁ and W₂ is —O— and another of W₁ and W₂ is —C(R₁₀R₁₁)—, or both W₁ and W₂ each independently is —C(R₁₀R₁₁)—, preferably wherein R₁₀ and R₁₁ each is H, Cl or F, and 1, 2, 3, 4 or 5 of Z₁, Z′₁, Z₂, Z′₂ and Z′₃ is S or —S⁻, i.e., (i) one of Z₁ and Z′₁ is —S⁻ or S, and another of Z₁ and Z′₁, Z₂, Z′₂ and Z′₃ each independently is O or —O⁻; one of Z₂ and Z′₂ is —S⁻ or S, and Z₁, Z′₁, another of Z₂ and Z′₂ and Z′₃ each independently is O or —O⁻; or Z′₃ is —S⁻ or S, and Z₁, Z′₁, Z₂ and Z′₂ each independently is O or —O⁻; (ii) one of Z₁ and Z′₁, and one of Z₂ and Z′₂, each independently is —S⁻ or S, and the other of Z₁, Z′₁, Z₂, Z′₂, and Z′₃, each independently is O or —O⁻; one of Z₁ and Z′₁, and Z′₃, each independently is —S⁻ or S, and the other of Z₁ and Z′₁, and Z₂ and Z′₂, each independently is O or —O⁻; one of Z₂ and Z′₂, and Z′₃, each independently is —S⁻ or S, and Z₁, Z′₁, and the other of Z₂ and Z′₂, each independently is O or —O⁻; Z₁ and Z′₁ each independently is —S⁻ or S, and Z₂, Z′₂ and Z′₃ each independently is O or —O⁻; or Z₂ and Z′₂ each independently is —S⁻ or S, and Z₁, Z′₁ and Z′₃ each independently is O or —O⁻; (iii) one of Z₁ and Z′₁, one of Z₂ and Z′₂, and Z′₃, each independently is —S⁻ or S, and the other of Z₁, Z′₁, Z₂ and Z′₂ each independently is O or —O⁻; Z₁ and Z′₁, and one of Z₂ and Z′₂, each independently is —S⁻ or S, and another of Z₂ and Z′₂, and Z′₃ each independently is O or —O⁻; Z₁ and Z′₁, and Z′₃, each independently is —S⁻ or S, and Z₂, Z′₂ are O or —O⁻; Z₂ and Z′₂, and one of Z₁ and Z′₁, each independently is —S⁻ or S, and another of Z₁ and Z′₁, and Z′₃ each independently is O or —O⁻; or Z₂ and Z′₂, and Z′₃, each independently is —S⁻ or S, and Z₁ and Z′₁ each independently is O or —O⁻; (iv) Z₁, Z′₁, one of Z₂ and Z′₂, and Z′₃, each independently is —S⁻ or S, and the other of Z₂ and Z′₂ is O or —O⁻; Z₂, Z′₂, one of Z₁ and Z′₁, and Z′₃, each independently is —S⁻ or S, and the other of Z₁ and Z′₁ is O or —O⁻; or Z₁, Z′₁, Z₂ and Z′₂ each independently is —S⁻ or S, and Z′₃ is O or —O⁻; or (v) Z₁, Z′₁, Z₂, Z′₂, and Z′₃ each independently is —S⁻ or S.

The compounds of the general formula I may be synthesized according to any technology or procedure known in the art, e.g., as described in detail in the Examples section hereinafter.

The compounds of the general formula I may have one or more asymmetric centers, and may accordingly exist as pairs of diastereoisomers. In cases a pair of diastereoisomers exists, the separation and characterization of the different diastereomers may be accomplished using any technology known in the art, e.g., HPLC.

The compounds of the general formula I are in the form of pharmaceutically acceptable salts, wherein B represents a pharmaceutically acceptable cation.

In certain embodiments, the cation B is an inorganic cation of an alkali metal such as, but not limited to, Na⁺, K⁺ and Li⁺.

In other embodiments, the cation B is ammonium (NH₄⁺) or is an organic cation derived from an amine of the formula R₄N⁺, wherein each one of the Rs independently is selected from H, C₁-C₂₂, preferably C₁-C₆ alkyl, such as methyl, ethyl, propyl, isopropyl, butyl, and the like, phenyl, or heteroaryl such as pyridyl, imidazolyl, pyrimidinyl, and the like, or two of the Rs together with the nitrogen atom to which they are attached form a 3-7 membered ring optionally containing a further heteroatom selected from N, S and O, such as pyrrolydine, piperidine and morpholine.

In further embodiments, the cation B is a cationic lipid or a mixture of cationic lipids. Cationic lipids are often mixed with neutral lipids prior to use as delivery agents. Neutral lipids include, but are not limited to, lecithins; phosphatidyl-ethanolamine; diacyl phosphatidylethanolamines such as dioleoyl phosphatidylethanolamine, dipalmitoyl phosphatidylethanolamine, palmitoyloleoyl phosphatidylethanolamine and distearoyl phosphatidylethanolamine; phosphatidyl-choline; diacyl phosphatidylcholines such as dioleoyl phosphatidylcholine, dipalmitoyl phosphatidylcholine, palmitoyloleoyl phosphatidylcholine and distearoyl phosphatidylcholine; fatty acid esters; glycerol esters; sphingolipids; cardiolipin; cerebrosides; ceramides; and mixtures thereof. Neutral lipids also include cholesterol and other 3β hydroxy-sterols. Other neutral lipids contemplated herein include phosphatidylglycerol; diacyl phosphatidylglycerols such as dioleoyl phosphatidylglycerol, dipalmitoyl phosphatidylglycerol and distearoyl phosphatidylglycerol; phosphatidylserine; diacyl phosphatidylserines such as dioleoyl- or dipalmitoyl phosphatidylserine; and diphosphatidylglycerols.

Examples of cationic lipid compounds include, without being limited to, Lipofectin® (Life Technologies, Burlington, Ontario) (1:1 (w/w) formulation of the cationic lipid N-[1-(2,3-dioleyloxy)propyl]-N,N,N-trimethylammonium chloride and dioleoylphosphatidyl-ethanolamine); Lipofectamine™ (Life Technologies, Burlington, Ontario) (3:1 (w/w) formulation of polycationic lipid 2,3-dioleyloxy-N-[2(spermine-carboxamido)ethyl]-N,N-dimethyl-1-propanamin-iumtrifluoroacetate and dioleoylphosphatidyl-ethanolamine), Lipofectamine Plus (Life Technologies, Burlington, Ontario) (Lipofectamine and Plus reagent), Lipofectamine 2000 (Life Technologies, Burlington, Ontario) (Cationic lipid), Effectene (Qiagen, Mississauga, Ontario) (Non liposomal lipid formulation), Metafectene (Biontex, Munich, Germany) (Polycationic lipid), Eu-fectins (Promega Biosciences, San Luis Obispo, Calif.) (ethanolic cationic lipids numbers 1 through 12: C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O₄S₂.8CF₃CO₂H, C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, C₄₁H₇₈NO₈P); Cytofectene (Bio-Rad, Hercules, Calif.) (mixture of a cationic lipid and a neutral lipid), GenePORTER® (Gene Therapy Systems, San Diego, Calif.) (formulation of a neutral lipid (Dope) and a cationic lipid) and FuGENE 6 (Roche Molecular Biochemicals, Indianapolis, Ind.) (Multi-component lipid based non-liposomal reagent).

### Neurodegenerative Diseases and Disorders

Alzheimer's disease (AD) is a progressive neuronal disease characterized by an irreversible neuronal damage which causes memory loss, impaired cognitive functions and loss of speech. The main features of AD include amyloid plaques constituted of the amyloid beta (Aβ), a 39-43 amino acid peptide, neurofibrillary tangles, consisting mainly of paired helical filaments of abnormally hyper-phosphorylated microtubule-associated t protein (Iqbal et al., 2005), oxidative stress and ROS formation (Pratico, 2008), and neuroinflammatory processes (Akiyama et al., 2000).

Aβ forms oligomers which give rise to fibrils. The role of Aβ is still ambiguous, although several possibilities have been suggested such as an antioxidant (Baruch-Suchodolsky and Fischer, 2009) or oxidant role (White et al., 2004), regulation of synaptic vesicle release (Abramov et al., 2009), and antimicrobial activity (Soscia et al., 2010).

Numerous studies indicate that Aβ binds Cu²⁺, Fe³⁺ and Zn²⁺ ions with high affinity (Garzon-Rodriguez et al., 1999; Faller and Hureau, 2009). Nuclear magnetic resonance (NMR) and electron spin resonance (ESR) data showed that Aβ peptide binds the metal-ion through three histidine residues, His6, His13, and His14 in a N₃O manner, while the oxygen atom origin is Tyr10, Glu5, or Asp1 (Faller and Hureau, 2009; Curtain et al., 2001; Karr et al., 2005).

High concentrations of Zn²⁺, Cu²⁺ and Fe²⁺ ions have been found in senile plaques, in histological section of AD patients (Lovell et al., 1998). In vitro it was shown that with the addition of Zn²⁺ at pH 7.4 and Cu²⁺ at pH 6.6 Aβ_{40/42} readily precipitated (Atwood et al., 1998). It was also demonstrated that these metal-ions can crosslink two Aβ peptides by His-M²⁺/M⁺-His intermolecular bridges (Faller, 2009; Miura et al., 2000) or lead to intermolecular cross-linked Aβ due to reaction of two Tyr10 tyrosyl radicals (Atwood et al., 2004). Moreover, Aβ was shown to be neurotoxic when incubated with Cu²⁺ or Fe²⁺ (Dai et al., 2009; Salvador et al., 2010).

A correlation was found between the increase of Fe/Cu/Zn ions concentrations in AD brains, 3-5 times more than in brains of healthy individuals, and the formation of Aβ plaques (Bush, 2003). Furthermore, the high concentrations of Fe/Cu ions were related to enhanced oxidative stress in AD (Jomova et al., 2010). Current therapies are not able to stop AD progression but offer only symptomatic relief and can, in the best case, slow cognitive decline (Lau and Brodney, 2008). These therapies attempt to address neurotransmitter defects (Francis et al., 1999), slow neurodegeneration (Simons et al., 2002), or treat inflammation and oxidative stress (Lim et al., 2000).

Other treatment strategies target Aβ production, aggregation, toxicity, or enhancement of Aβ degradation. These strategies include γ-secretase inhibitors that reduce Aβ production (Panza et al., 2010), neprilysin that promote Aβ degradation (Selkoe, 2001), β-sheet breakers which prevent or slow oligomers/fibril formation (Bartolini et al., 2007), humanized antibodies against Aβ peptide which reduce Aβ load (Bombois et al., 2007), and metal-ion chelators that block Aβ aggregation (Scott and Orvig, 2009).

Several β-sheet inhibitors have been reported (Bartolini et al., 2007); however, these inhibitors do not address the increasing age-related metal-ion concentration that is a key factor for Aβ oligomerization, fibril formation, and oxidative stress.

Metal-ion chelators such as clioquinol (PBT1) (Ritchie et al., 2003) and PBT2 (8-hydroxy quinoline analogue) (Cherny et al., 2008) are moderate affinity binding chelators considered to be ionophores. The mode of action of clioquinol and PBT2 is denoted as metal-protein attenuating compounds (MPAC) (Ritche et al., 2004). Clioquinol and PBT2 lowered Aβ load both in in vitro and in vivo studies (Adlar et al., 2008; LeVine et al., 2009); however, clioquinol failed phase II clinical trials. PBT2 showed better in vivo results than clioquinol in reducing insoluble Aβ in transgenic mice brain (Cherny et al., 2008). Other metal-ion chelators such as deferiprone (Green et al., 2010) and N1,N2-bis(pyridine-2-yl-methyl)-ethane-1,2-diamine (Lakatos et al., 2010) have been recently shown to redissolve Aβ_{40/42}-metal-ion aggregates. Subsequent to metal-ion chelators disassembly of Aβ_{40/42}-M²⁺ aggregates, the free Aβ peptide may be degraded by proteases (Selkoe, 2001) or cleared to the bloodstream (Zlokovic, 2004).

AD is a multi-parameter disease involving highly complex biochemical mechanisms. Therefore, an AD disease modifying drug is preferentially a multifunctional one simultaneously addressing several drug targets. An ideal drug candidate, for instance, lowers the Aβ load in the brain, and in addition serves as MPAC and an antioxidant (Doraiswamy and Finefrock, 2004).

Nucleotide analogues are natural metal-ion chelators (Sigel and Griesser, 2005). For instance, ATP forms stable complexes with various divalent metal ions (e.g., Fe²⁺, Mg²⁺, Zn²⁺ and Cu²⁺), of which the most stable is the Cu²⁺-ATP complex (log K 6.34) that is 1.2-2.5 orders of magnitude more stable than the other complexes (Sigel and Griesser, 2005). Furthermore, ATP and GTP were shown to be the dominant ligands affecting the chelation of iron and transferring it into the cell before it is incorporated into heme and ferritin (Weaver, 1989; Weaver et al., 1993). Related observations were made for the Cu²⁺-ion (Barnea et al., 1991).

Previously, we investigated nucleotides and phosphate analogues as potential antioxidants. Specifically, we found that ATP-γ-S proved a most potent antioxidant inhibiting OH radical production in the Fe²⁺/H₂O₂ system with IC₅₀ of 10 μM (being 100 and 20 times more active than ATP and the potent antioxidant Trolox, respectively). Likewise, nucleotides and phosphates (e.g., ATP, ADP, and thiophosphate) proved potent antioxidants in Cu⁺/Cu²⁺—H₂O₂ systems (Richter and Fischer, 2006; Baruch-Suchodolsky and Fischer, 2008). Modification of a nucleotide by a terminal thiophosphate moiety (e.g. ATP-γ-S and ADP-β-S) resulted in significantly enhanced antioxidant activity as compared to that of the corresponding parent compound. Our previous findings demonstrating the antioxidant activity of nucleoside 5′-phosphorothioate analogues encouraged us to evaluate them as biocompatible and water-soluble agents for the dissolution of Aβ-M²⁺ aggregates.

As shown in the Examples section hereinafter, mononucleoside 5′-phosphorothioate of the general formula I as defined above are capable of protecting primary cortical neuronal cells from damage caused by FeSO₄ and from Aβ₄₂ insult. As particularly shown, APCPP-γ-S protected primary cortical neuronal cells from damage caused by FeSO₄ with IC₅₀ values of 40 nM as compared to ATP-γ-S (IC₅₀ 10 nM), and furthermore, protected primary neurons from Aβ₄₂ insult with IC₅₀ of 200 nM as compared to ATP-γ-S (IC₅₀ 800 nM). These results are consistent with our preliminary results in PC12 cells under oxidative stress (IC₅₀ value obtained for APCPP-γ-S was 0.16 μM). Interestingly, the neuroprotection activity of APCPP-γ-S is due to neither P2Y₁R nor P2Y₂R activation, but may be due to P2Y₁₁ receptor activation (EC₅₀ value of 1 μM). The studies described herein also show that APCPP-γ-S is metabolically stable with no significant degradation after 3 h in mouse blood or brain and liver homogenates; and that upon IV administration to mice, 64% of the APCPP-γ-S injected remained in blood after 90 min. APCPP-γ-S was further found to be of minor toxicity and reduced PC12 cell viability by only 25% at 1000 μM.

It is therefore concluded that mononucleoside 5′-phosphorothioate of the general formula I as defined above, such as APCPP-γ-S, are highly effective neuroprotectants protecting primary neurons from Aβ toxicity and oxidative stress, as well as highly effective agents for dissolution of Aβ aggregates, and effective chelators of Zn/Cu/Fe ions. As specifically shown with respect to APCPP-γ-S, these compounds act not only as metal-ion chelators but also as radical scavengers protecting neurons also through the activation of P2Y receptors. It is expected that other mononucleoside 5′-phosphorothioate of the present invention, such as APCP-α,α′,β,β′-tetra-S and UPCP-α,α′,β,β′-tetra-S exemplified herein, as well as dinucleoside 5′-phosphorothioate of the present invention such as APCPA-α,α′,β,β′-tetra-S and UPCPU-α,α′,β,β′-tetra-S, will have a similar activity.

The activity of the mono- and di-nucleoside 5′-phosphorothioate of the present invention, and in particular that of APCPP-γ-S, makes these compounds attractive candidates for treatment of neurodegenerative diseases or disorders such as Alzheimer's disease (AD), closely associated with the formation of Aβ aggregates, as well as Parkinson's disease, Huntington's disease, amyotrophic lateral sclerosis (ALS), and Creutzfeldt-Jakob disease. A protocol for testing the efficacy of APCPP-γ-S in a mouse model of AD is provided in the Examples section.

### Osteoarthritis

APCPP-γ-S and APPCCl₂P-α-S were found to be NPP1 inhibitors. These analogues were not hydrolyzed by NPP1, 3 and ectonucleotidases, NTPDase1, 2, 3, 8 (<5% hydrolysis), and barely affected the activity of NTPDase1, 2, 3, 8 and NPP3. APCPP-γ-S and APPCCl₂P-α-S inhibited pnp-TMP hydrolysis by NPP1 and NPP3 by 90-100% and 33-44%, respectively, and the hydrolysis of ATP by NTPDase1, 2, 3, 8, by 0-40%. These analogues showed only weak activity (EC₅₀>10 μM) as P2Y_{1,2,11} receptor agonists. APPCCl₂P-α-S was found to be the most potent NPP1 inhibitor currently known, with Kᵢ of 20 nM and IC₅₀ of 0.39 μM. Yet, it also inhibited the hydrolysis of ATP by NTPDase1 by 58%, NTPDase3 by 40% and NPP3 by 33%. APCPP-γ-S (isomer A) was found to be a selective NPP1 inhibitor, with Ki 685 nM and IC₅₀ 0.57 μM, which only slightly inhibited the hydrolysis of ATP by NPP3 (38%), NTPDase1 (0%) and NTPDase3 (22%). Indeed, we found that APPCCl₂P-α-S had a 50-times higher affinity to Zn²⁺ ions as compared to ATP (log K 6.5), making it a better competitor for the zinc-containing NPP1 catalytic site. Preliminary toxicity studies with APPCCl₂P-α-S at PC12 cells indicated a high safety profile. At 100 and 1000 μM, 100% and 75% of the cells remained viable, respectively. APPCCl₂P-α-S when administered IV to mice could be detected in blood (by HPLC) even after 3 h, demonstrating its relative in-vivo stability.

As surprisingly found and shown the Examples section, APPCCl₂P-α-S, which si the most potent NPP1 inhibitor currently known, is further effective in reducing ATP hydrolysis and PPi and CPPD formation in human MVs, chondrocytes and cartilage.

### Pharmaceutical Compositions

In another aspect, the present invention thus provides a pharmaceutical composition comprising a mono- or dinucleoside 5′-phosphorothioate of the general formula I as defined in any one of the embodiments above, but excluding those compounds excluded by means of proviso, or a diastereomer or mixture of diastereomers thereof, and a pharmaceutically acceptable carrier or diluent. In particular embodiments, the pharmaceutical composition of the invention comprises, as an active agent, a mono- or dinucleoside 5′-phosphorothioate of the general formula I selected from those exemplified herein, preferably APCPP-γ-S, APPCP-α-S or APPCCl₂P-α-S.

The pharmaceutical compositions provided by the present invention may be prepared by conventional techniques, e.g., as described in Remington: The Science and Practice of Pharmacy, 19^{th} Ed., 1995. The compositions can be prepared, e.g., by uniformly and intimately bringing the active agent, i.e., the compound of the general formula I as defined above, into association with a liquid carrier, a finely divided solid carrier, or both, and then, if necessary, shaping the product into the desired formulation. The compositions may be in liquid, solid or semisolid form and may further include pharmaceutically acceptable fillers, carriers, diluents or adjuvants, and other inert ingredients and excipients. In one embodiment, the pharmaceutical composition of the present invention is formulated as nanoparticles.

The pharmaceutical compositions of the invention can be formulated for any suitable route of administration, but they are preferably formulated for parenteral, e.g., intravenous, intraarterial, intramuscular, intraperitoneal, intrathecal, intrapleural, intratracheal, subcutaneous, transdermal, sublingual, inhalational, or oral administration. The dosage will depend on the state of the patient, and will be determined as deemed appropriate by the practitioner.

The pharmaceutical composition of the invention may be in the form of a sterile injectable aqueous or oleagenous suspension, which may be formulated according to the known art using suitable dispersing, wetting or suspending agents. The sterile injectable preparation may also be a sterile injectable solution or suspension in a non-toxic parenterally acceptable diluent or solvent. Acceptable vehicles and solvents that may be employed include, without limiting, water, Ringer's solution and isotonic sodium chloride solution.

The pharmaceutical compositions of the invention, when formulated for administration route other than parenteral administration, may be in a form suitable for oral use, e.g., as tablets, troches, lozenges, aqueous, or oily suspensions, dispersible powders or granules, emulsions, hard or soft capsules, or syrups or elixirs. Compositions intended for oral use may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active agent in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

Pharmaceutical compositions according to the invention, when formulated for inhalation, may be administered utilizing any suitable device known in the art, such as metered dose inhalers, liquid nebulizers, dry powder inhalers, sprayers, thermal vaporizers, electrohydrodynamic aerosolizers, and the like.

The pharmaceutical compositions of the invention may be formulated for controlled release of the active agent. Such compositions may be formulated as controlled-release matrix, e.g., as controlled-release matrix tablets in which the release of a soluble active agent is controlled by having the active diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other configurations, the compositions comprise the active agent formulated for controlled release in microencapsulated dosage form, in which small droplets of the active agent are surrounded by a coating or a membrane to form particles in the range of a few micrometers to a few millimeters.

Another contemplated formulation is a depot system based on a biodegradable polymer, wherein as the polymer degrades, the active agent is slowly released. The most common class of biodegradable polymers is the hydrolytically labile polyesters prepared from lactic acid, glycolic acid, or combinations of these two molecules. Polymers prepared from these individual monomers include poly(D,L-lactide) (PLA), poly(glycolide) (PGA), and the copolymer poly(D,L-lactide-co-glycolide) (PLG).

The pharmaceutical composition of the present invention may be used for treatment of a neurodegenerative disease or disorder, e.g., AD, Parkinson's disease, Huntington's disease, ALS, and Creutzfeldt-Jakob disease, as well as osteoarthritis or CPPD deposition

In still another aspect, the present invention relates to a method for treatment of a neurodegenerative disease or disorder in an individual in need thereof, comprising administering to said individual a therapeutically effective amount of a mono- or dinucleoside 5′-phosphorothioate of the general formula I as defined above, or a diastereomer or mixture of diastereomers thereof.

In yet another aspect, the present invention relates to a method for treatment of osteoarthritis or CPPD deposition disease in an individual in need thereof, comprising administering to said individual a therapeutically effective amount of a mono- or dinucleoside 5′-phosphorothioate of the general formula I as defined in any one of the embodiments above, but excluding those compounds excluded by means of proviso, or a diastereomer or mixture of diastereomers thereof.

The invention will now be illustrated by the following non-limiting Examples.

### EXAMPLES

### Experimental

### Materials and Methods

Reactions were performed in oven dried flasks under Ar atmosphere. Tetrakis(acetonitrile)-copper(I) hexafluorophosphate (Cu(CH₃—CN)₄PF₆), BCA disodium salt, trisodiumthiophosphate, DMPO, and sodium triphosphate were purchased from Sigma-Aldrich Chemical Co. DBU and 3-hydroxypropionitrile were purchased from Sigma-Aldrich and distilled under reduced pressure before use. Clioquinol was purchased from Fisher scientific. Adenosine 5′-[γ-thio]-triphosphate, adenosine 5′-[β-thio]diphosphate, guanosine 5′-[β-thio]diphosphate, and guanosine 5′-[γ-thio]triphosphate were synthesized according to literature (Kowalska et al., 2007). Deuterated solvents—D₂O, DMSO-d₆, Tris-d₁₁, NaOD, and DCl—were purchased from Cambridge Isotope Laboratories, Inc. CHCl₃ was distilled over P₂O₅.

Cu(CH₃CN)₄PF₆ was purified before use by dissolving the salt in acetonitrile (HPLC grade) and filtering the insoluble Cu²⁺ salt by a nylon syringe 0.45 μm filter. The filtrate was deaerated with argon stream. The concentration of the Cu⁺ salt was determined by UV spectroscopy by the addition of the specific Cu⁺ indicator, BCA (ε₅₆₂=7700 M⁻¹) (Brenner and Harris, 1995). Crude Aβ₂₈ was purchased from Sigma-Aldrich and was purified by HPLC over a Chromolith performance RP-18E column, 100×4.6 mm, applying alinear gradient of 13% to 45% B in 30 min (A is 0.1% TFA in H₂O and B is 3:1 acetonitrile:A). Solution of the purified peptide was filtered over a PVDF 0.45 μm filter and the peptide purity was determined by ¹H-NMR, RP-HPLC, and MALDI-TOF MS. The concentration of soluble Aβ₂₈/Aβ₄₀ was based on UV measurements using the extinction coefficient of tyrosine residue (ε=1280 M⁻¹ at 280 nm). >95% Pure Aβ_{40/42} TFA salt was purchased from GL-Biochem (Shanghai) and kept at −20° C. Aβ₄₀ was weighted and dissolved in 10 mM NaOH and then freeze-dried (Fezoui et al., 2000). The freeze-dried peptide was dissolved in PBS (10 mM; 2.7 mM potassium chloride and 137 mM sodium chloride), and the concentration of the mixture was determined by UV. Aβ₄₂ was weighted and dissolved in 10 mM NaOH sonicated for 3 min and then freeze-dried.

The concentration of the spin trap, DMPO, was determined by UV spectroscopy (ε₂₂₈ nm=8000 M⁻¹) after purification by active charcoal. Purified DMPO was stored at −18° C. subsequent to deaeration with argon stream. Analysis of OH radicals produced in Cu⁺ and Fe⁺²—H₂O₂/tested compound systems were performed by solution ESR spectroscopy using a Bruker ER100d X-band spectrophotometer.

UV spectra were measured using a Shimadzu UV-VIS2401pc instrument. ¹H and ³¹P-NMR spectra were measured using a Bruker AC-200 (200 and 81 MHz for ¹H and ³¹P NMR, respectively), DMX-600 (600 and 243 MHz for ¹H and ³¹P NMR, respectively), or Avance III-700 (700 MHz for ¹H NMR) spectrometers. DLS measurements were performed using a Malvern Zetasizer Nano ZS Instrument (Worcestershire, UK) at 25° C. TEM images were obtained by Tecnai G2 microscope, FEI Co (Hillsboro, Oreg., USA).

Flash chromatography (silica-gel and C₁₈ reverse phase) was done using a Biotage SP1 instrument. ¹H, ¹³C, and ³¹P-NMR spectra were measured using Bruker AC-200 (200, 50 and 81 MHz for ¹H, ¹³C, and ³¹P NMR), and Bruker DMX-600 (600, 150 and 243 MHz for ¹H, ¹³C, and ³¹P-NMR) machines. Mass spectra analyses were performed on an ESI Q-TOF micro instrument (Waters, UK) and a high resolution MS-MALDI-TOF spectra with autoflex TOF/TOF instrument (Bruker, Germany). Purification of the nucleotides was achieved on a liquid chromatography (LC) (Isco UA-6) system with a Sephadex DEAE-A25 column, which was swelled in 1 M NaHCO₃ in the cold for 1 day.

### Titration of Aβ₂₈-Cu⁺ Complex by Various Chelators Monitored by ¹H-NMR

Stock solutions of 8 mM nucleotides were prepared in D₂O and pD was adjusted to 7 by DCl or NaOD. Clioquinol was dissolved in DMSO-d₆ (80 mM). Stock solutions were deaerated by a stream of Ar.

Pure Aβ₂₈ TFA salt >95% (1.3 mg, 5.5×10⁴ mmol) was dissolved in D₂O and freeze-dried for two times to exchange H₂O molecules with D₂O. The dry substance was dissolved in 10 mM Tris-d₁₁ (40 μl) to obtain 1 mM solution at pD 7, pH adjustment was achieved with NaOD or DCl. This sample was transferred via a syringe to an argon flashed NMR tube, covered with a rubber septum. 4 mM Cu⁺ (0.25 eq, 25 μl) stock solution was added to the Aβ₂₈ solution until the ratio of Aβ₂₈-Cu⁺ reached 1:1 and the mixture became cloudy. The addition of Cu⁺ solution to Aβ₂₈ was monitored by ¹H-NMR spectra 700 MHz (96 scans). The final Aβ₂₈-Cu⁺ concentration was 0.8 mM in 500 μl then, 50 μl, 1 eq of the tested nucleotide solution was added each time via syringe. At the end of the titration, the acetonitrile concentration was 17.2% (v/v).

### ¹H/³¹P-NMR Monitored Cu⁺ Titrations of ADP-β-S

A solution of ADP-β-S (9 mM, 40 μl, pD 8.2 in D₂O) was injected to an Ar purged NMR tube and ¹H/³¹P-NMR spectra were measured. Then, a solution of 6 mM Cu(CH₃CN)₄PF₆ in CD₃CN was added. After each addition H/³¹P-NMR spectra were measured. Overall, 80 μl of Cu⁺ solution was added to the NMR tube (0.87 eq).

Determination of Free Thiol in Thiophosphate and GDP-β-S with Ellman's Reagent Monitored by UV-Vis

A solution of 1 mM Cu(CH₃CN)₄PF₆ (200 μl) was added to Aβ₂₈ (0.118 mM, 169 μl) in 1 mM Tris buffer (pH 7.4). After 30 min 10 mM thiophosphate or GDP-β-S (100 μl) was added. After an additional 1 h, 10 mM Ellman's reagent (10 μl) in methanol was added to give a total volume of 2 ml. The final concentrations of reaction compounds were: 0.1 mM Cu(CH₃CN)₄PF₆, 0.1 mM Aβ₂₈, 0.5 mM thiophosphate or GDP-β-S and, 0.05 mM Ellman's reagent. Oxidation of thiophosphate compounds was monitored by UV spectroscopy (at the wavelength range of 275-500 nm). The control solution contained only the thiophosphate compound and Ellman's reagent in Tris buffer.

### DLS Measurements

PBS buffer, Aβ₄₀, chelator, Zn(NO₃)₂, and Cu(NO₃)₂ solutions were filtered through a 0.45 μM PVDF syringe filter. 496 μM Aβ₄₀ solution in 10 mM PBS (pH 7.4) and 2 mM Zn(NO₃)₂ solution were mixed to obtain 200 μM Aβ₄₀-Zn²⁺ (1:1 ratio) of a cloudy solution. Similarly, 2 mM Cu(NO₃)₂ solution was added to 496 μM Aβ₄₀ in 10 mM PBS (pH 6.6). Aβ₄₀-Zn²⁺/Cu²⁺ (10 μl) solution was transferred to Eppendorf tubes containing 10 mM PBS buffer (64 or 67 μl) followed by the addition of 3 and 6 eq of 2 mM nucleotide (6 or 3 μl) solution and incubation for 45 min at RT. The resulting mixture was incubated for another 30 min at RT and DLS data were then collected in a 70 μl disposable cuvette. The final sample concentrations were 25 μM Aβ₄₀, 25 μM Zn²⁺ or Cu²⁺, and 150 or 75 μM nucleotide.

### TEM Measurements

TEM sample was prepared from 200 μM Aβ-M²⁺ 1:1 solution (pH 7.4 or 6.6 for Aβ₄₀-Zn²⁺ or Aβ₄₀-Cu²⁺, respectively) incubated in a rotary shaker for 9 days at RT. A cloudy mixture with massive sediments was obtained. TEM samples were prepared by diluting the stock solutions with PBS to a concentration of 25 μM with or without APCPP-γ-S. In this way four samples were obtained: (i) 25 μM Aβ₄₀-Cu²⁺; (ii) 25 μM Aβ₄₀-Cu²⁺ containing 150 μM APCPP-γ-S; (iii) 25 μM Aβ₄₀-Zn²⁺; and (iv) 25 μM Aβ₄₀-Zn²⁺ containing 150 μm APCPP-γ-S. The mixtures were incubated in a rotary shaker for 7 h and after vortexing, a sample was transferred to a gold grid, for Aβ₄₀-Cu²⁺, or a copper grid for Aβ₄₀-Zn²⁺. The wet grid was left to dry at RT overnight.

### Disaggregation of Aβ₄₂-Cu²⁺/Zn²⁺ Complexes by Nucleotides Monitored by a Turbidity Assay

Aβ₄₂ was weighted and dissolved in 10 mM NaOH, sonicated for 3 min and then freeze-dried. The freeze-dried Aβ₄₂ was dissolved in 50 mM Tris-HCl (pH 7.4). The mixture was split and the pH of one of the samples was adjusted to 7.4 and the second sample to 6.6 by addition of 200 μM HCl. Two 300 μM Aβ₄₂ mixtures were obtained. From these mixtures, controls and Aβ₄₂-M²⁺ aggregates were prepared by the addition of 1 mM Zn(NO₃)₂ or Cu(NO₃)₂ in DDW: 1. 200 μM Aβ42 (pH 7.4); 2. 200 μM Aβ₄₂ (pH 6.6); 3.4×200 μM Aβ₄₂-Zn²⁺ (pH 7.4); 4.4×200 μM Aβ₄₂-Cu²⁺ (pH 6.6). The mixtures were left at RT for 2 h to form aggregates. 1 mM EDTA, ADP-β-S, and APCPP-γ-S in DDW were added to Aβ₄₂-Zn²⁺/Cu²⁺ aggregates, then the mixtures were diluted with buffer to give the following mixtures: (i) 50 μM Aβ₄₂-Cu²⁺/Zn²⁺; (ii) 50 μM Aβ₄₂-Cu²⁺/Zn²⁺ containing 150 μM chelator (3 eq or) 300 μM chelator (6 eq) final volume 100 μl. The mixtures left at RT for 30 min before measurements, done in duplicate. 80 μl sample was taken for measurements in a quartz cuvette.

### Titration of Aβ₄₀-Cu⁺ Complex by Nucleotides Monitored by ¹H-NMR

¹H-NMR spectrum of 0.25 mM Aβ₄₀ (concentration determined by UV) in 10 mM TRIS-d₁₁ (500 μl, pD 11) was measured at 278 K. The pD was adjusted to 7.8 by the addition of 0.1 N DCl (33 μl), and ¹H-NMR spectrum was measured (700 MHz, 80 scans), as well as after each of the following additions: (A) 8.3 mM Cu⁺ (15 μl, 1 eq); (B) 6 mM compound 7 (21 μl, 1 eq); (C) 6 mM compound 7 (42 μL, 3 eq); (D) of 6 mM APCPP-γ-S (62.4 μl, 6 eq); (E) of 6 mM APCPP-γ-S (62.4 μl, 9 eq). At the end of the titration, the acetonitrile concentration was 2% (v/v) in 735.8 μl, pD 8.4.

### ESR OH Radical Assay

ESR settings for OH radicals detection were as follows: microwave frequency, 9.76 GHz; modulation frequency, 100 KHz; microwave power, 6.35 mW; modulation amplitude, 1.2 G; time constant, 655.36 ms; sweep time 83.89 s; and receiver gain 2×10⁵ in experiments with Cu⁺ and Fe²⁺.

1 mM Cu(CH₃CN)₄PF₆ in acetonitrile (10 μl) or 1 Mm FeSO₄ (10 μl) were added to 5-500 μM tested compound (10 μl) solutions. All final solutions of Cu(CH₃CN)₄PF₆ contained 10% v/v acetonitrile. Afterwards, 1 mM Tris buffer, pH 7.4, (10 μl) was added to the mixture. After mixing for two seconds, 100 mM DMPO (10 μl) were quickly added followed by the addition of 100 mM H₂O₂ (10 μl). The final sample pH values for the Cu⁺ and Fe²⁺ systems ranges between 7.2-7.4. Each ESR measurement was performed 150 sec after the addition of H₂O₂. All experiments were performed at RT, in a final volume of 100 μl.

### Evaluation of the Resistance of Particular Analogues to Hydrolysis by NPP1,3

The percentage of hydrolysis of the analogues tested by human NPP1,3 was evaluated as follows: 67 μg or 115 μg of human NPP1 or NPP3 extract, respectively, was added to 0.579 ml the incubation mixture (1 mM CaCl₂, 200 mM NaCl, 10 mM KCl and 100 mM Tris, pH 8.5) and pre-incubated at 37° C. for 3 min. Reaction was initiated by the addition of 0.015 ml of 4 mM analogue; and was stopped after 30 min or 1 h for NPP1 or NPP3, respectively, by adding 0.350 ml ice-cold 1 M perchloric acid. These samples were centrifuged for 1 min at 10,000 g. Supernatants were neutralized with 140 μl 2 M KOH in 4° C. and centrifuged for 1 min at 10,000 g. The reaction mixture was filtered and freeze-dried.

Each sample was dissolved in 200 μl HPLC-grade water and 20 μl sample was injected onto an analytical HPLC column (Gemini analytical column (5μ C-18 557 110A; 150 mm×4.60 mm)), using isocratic elution with 85%-97% 100 mM TEAA (pH 7) and 15%-3% AcN, flow rate 1 ml/min. The percentage of the buffer and AcN depended on the chemical structure of the substrate.

The hydrolysis rates of all analogues by NPP1 or NPP3 were determined by measuring the change in the integration of the HPLC peaks for each analogue over time vs. control. The percentage of compound degradation was calculated versus control, to take into consideration the degradation of the compounds due to the addition of acid to stop the enzymatic reaction. Therefore, each of the samples was compared to a control which was transferred to acid, but to which no enzyme was added. The percentage of degradation was calculated from the area under the curve of the nucleoside monophosphate peak, after subtraction of the control, which is the amount of the nucleoside monophosphate peak formed due to chemical acidic hydrolysis.

### Evaluation of the Resistance of Particular Analogues to Hydrolysis by NTPDase1,2,3,8

The percentage of hydrolysis of the analogues tested by human NTPDase-1,2,3,8 was evaluated as follows: 2.8 μg or 4.3 μg of human NTPase1 or NTPDase2 extract, respectively, was added to 0.579 ml the incubation mixture (10 mM CaCl₂ and 160 mM Tris, pH 7.4) and pre-incubated at 37° C. for 3 min. The reaction was initiated by the addition of 0.012 ml of 4.24 mM analogue solution; and was stopped after 1 h for NTPase1,2,3,8, by adding 0.350 ml ice-cold 1 M perchloric acid. These samples were centrifuged for 1 min at 10,000 g. Supernatants were neutralized with 140 μl 2 M KOH in 4° C. and centrifuged for 1 min at 10,000 g. The reaction mixture was filtered and freeze-dried.

Each sample was dissolved in 200 μl HPLC-grade water and a 20 μl sample was injected to an analytical HPLC column (Gemini analytical column (5μ C-18 557 110A; 150 mm×4.60 mm)), and eluted using isocratic elution with 78%-97% 100 mM TEAA (pH 7) and 22%-3% AcN, flow rate 1 ml/min. The percentage of the buffer and AcN depended on the chemical structure of the substrate.

The hydrolysis rates of all analogues by NTPDase-1,2,3,8 were determined by measuring the change in the integration of the HPLC peaks for each analogue over time vs. control. The percentage of compound degradation was calculated vs. control, to take into consideration the degradation of the compounds due to the addition of acid to stop the enzymatic reaction. Therefore, each of the samples was compared to a control which was transferred to acid, but to which no enzyme was added. The percentage of degradation was calculated from the area under the curve of the nucleoside monophosphate peak, after subtraction of the control, which is the amount of the nucleoside monophosphate peak formed due to chemical acidic hydrolysis.

### Inhibition of NTPDase Activity Assays

Activity was measured as previously described (Kukulski et al., 2005) in 0.2 ml of incubation medium Tris-Ringer buffer (in mM, 120 NaCl, 5 KCl, 2.5 CaCl₂, 1.2 MgSO₄, 25 NaHCO₃, 5 glucose, 80 Tris, pH 7.3) at 37° C. with or without the analogue tested (final concentration 100 μM), and with or without 100 μM ATP (for NTPDases) or AMP (for ecto-5′-nucleotidase) as a substrates. The analogues were added alone when tested as potential substrate, and with ATP when tested for their effect on nucleotide hydrolysis. NTPDases protein extracts were added to the incubation mixture and pre-incubated at 37° C. for 3 min. The reaction was initiated by the addition of substrate (ATP and/or the analogue tested); and was stopped after 15 min with 50 μl of malachite green reagent. The released inorganic phosphate (Pi) was measured at 630 nm according to Baykov et al. (1988).

### Inhibition of NPP Activity Assays

Evaluation of the effect of adenosine-5′-tetrathio bisphosphonate, di-adenosine 5′,5″-tetrathiobisphosphonate, and ADP-β-S on human NPP1 and 3 activity was carried out either with pnp-TMP or ATP as the substrate (Belli and Goding, 1994). Pnp-TMP hydrolyses were carried out at 37° C. in 0.2 ml of the following incubation mixture: in mM, 1 CaCl₂, 130 NaCl, 5 KCl and 50 Tris, pH 8.5, with or without the analogue tested and/or substrates. Substrates and analogues were all used at the final concentration of 100 μM. Recombinant human NPP1 or NPP3 cell lysates were added to the incubation mixture and pre-incubated at 37° C. for 3 min. The reaction was initiated by the addition of the substrate. For pnp-TMP hydrolysis, the production of para-nitro-phenol was measured at 310 nm, 15 min after the initiation of the reaction.

Evaluation of the activity of human NPP1 and NPP3 with ATP (Sigma-Aldrich, Oakville, ON, Canada) and each one of the analogues tested was carried out at 37° C. in 0.2 ml of the following mixture: (in mM) 1 CaCl₂, 140 NaCl, 5 KCl, and 50 Tris, pH 8.5; (Sigma-Aldrich, Oakville, ON, Canada). Human NPP1 or NPP3 extract was added to the reaction mixture and pre-incubated at 37° C. for 3 min. The reaction was initiated by addition of ATP or one of the analogues at a final concentration of 100 μM; and was stopped after 20 min by transferring a 0.1 ml aliquot of the reaction mixture to 0.125 ml ice-cold 1 M perchloric acid (Fisher Scientific, Ottawa, ON, Canada). The samples were centrifuged for 5 min at 13,000×g. Supernatants were neutralized with 1 M KOH (Fisher Scientific, Ottawa, ON, Canada) at 4° C. and centrifuged for 5 min at 13,000×g. An aliquot of 20 ml was separated by reverse-phase HPLC to evaluate the degradation of ATP and the level of the analogue tested using a SUPELCOSIL™ LC-18-T column (15 cm×4.6 mm; 3 mm Supelco; Bellefonte, Pa., USA) with a mobile phase composed of 25 mM TBA, 5 mM EDTA, 100 mM KH₂PO₄/K₂HPO₄, pH 7.0 and 2% methanol at a flow rate of 1 ml/min.

### Example 1

### Synthesis of adenosine 5′-[Pγ-thio]-α,β-methylenetriphosphate, APCPP-γ-S

As depicted in Scheme 1, CDI (143 mg, 0.88 mmol) was added at RT to a solution of ADP-α,β-methylene (75 mg, 0.17 mmol) in dry DMF (2 ml) in a flamed-dried, nitrogen-flushed two-necked round bottom flask and stirred for 3 h. TLC on a silica gel plate (isopropanol:NH₄OH:H₂O 11:2:7) indicated the disappearance of the starting material and the formation of a less polar product. Dry MeOH (28 μl) was added and the reaction was stirred for 8 min, ZnCl₂ (354 mg, 2.65 mmol) was added followed by thiophosphate (Bu₃NH⁺)₂ salt and tri-n-octyl amine and ADP-α,β-methylene trioctylammonium salt (190 mg, 1.06 mmol) in dry DMF (1 ml). The reaction was stirred for 3 h, and EDTA (1.18 g, 3.17 mmol) in distilled water (15 ml) was then added to the solution at RT. After a few minutes 1 M TEAB was added until the pH of the solution changed to pH˜8. The colorless clear solution was freeze-dried overnight. The resulting residue was separated on an activated Sephadex DEAE-A25 column (0-0.4 M NH₄HCO₃; total volume 700 ml). The relevant fractions were collected, freeze-dried, and excess NH₄HCO₃ was removed by repeated freeze-drying with deionized water to yield the product as a yellow powder. Analogue APCPP-γ-S was separated on a semipreparative reverse phase Gemini 5u column and isocratic elution with 96:4 (TEAA buffer pH 7:CH₃CN) over 20 min at a flow rate of 5 ml/min. Retention time: 9.0 min (20 mg, 19%). Finally, the purified analogue was passed through a Sephadex-CM C-25 Na⁺ form column to exchange triethylammonium ions for Na⁺. APCPP-γ-S TEAA salt: ¹H-NMR: 8.58 (s, H8), 8.26 (s, H2), 6.09 (J=5.8 Hz, H1′), 4.90 (m, H2′), 4.56 (m, H3′), 4.36 (m, H4′), 4.20 (m, H5′), 3.20 (m, Et₃N), 2.48 (t, J=20 Hz, CH₂), 2.00 (s, CH₃CO₂H), 1.30 ppm (m) Et₃N. ³¹P-NMR: 39.02 (d, J=32 Hz, Pγ), 18.15 (d, J=9 Hz, Pα), 6.88 (dd, J=9 Hz, J=32 Hz, Pβ) ppm. MS-ES m/z: 519 (M-H)−. HRMS-FAB (negative) m/z: calculated for C₁₁H₁₇N₅O₁₁P₃S²⁻: 519.9853. found: 519.982.

### Example 2

### Synthesis of adenosine 5′-[Pα-thio]-β,γ-methylenetriphosphate, APPCP-α-S

As depicted in Scheme 2, a solution of 2-chloro-4H-1,3,2-benzodioxaphosphorin-4-one (98 mg, 0.48 mmol) in anhydrous DMF (0.75 ml) was added via syringe to a solution of 2′,3′-orthoformate-protected adenosine (2a) (100 mg, 0.32 mmol) and anhydrous pyridine (250 μl) in 0.5 ml of anhydrous DMF at 0° C. under nitrogen. After the resulting solution was stirred at RT for 1 h, tributylamine (500 μl) was added, followed by a solution of bis(tributylammonium)methylenediphosphonate (68 mg, 0.38 mmol) in anhydrous DMF (0.5 ml). The reaction mixture was stirred at RT for 2 h, and then sulfur (21 mg, 0.64 mmol) was added at 0° C. The solution color changed to orange and afterward to brown. After being stirred at room temperature for 1.5 h, the mixture was dripped into a cold 1 M TEAB solution (10 ml) until pH≈7 was attained. The resulting mixture was stirred at room temperature for 30 min. During that time the color of the solution changed to yellow. The solution was extracted (2×10 ml) with ether. The aqueous phase was freeze-dried twice. The product was then deprotected by addition of 10% HCl until pH 2.3 was attained, and the mixture was stirred for 3 h. Afterward 24% NH4OH was added to give pH≈9, and the mixture was stirred for another 45 min and freeze-dried overnight.

The crude residue was separated on a DEAE-Sephadex A25 column with a linear gradient of ammonium bicarbonate (from 0.1 to 0.4 M ammonium bicarbonate, total gradient volume 600 ml). The relevant fraction was freeze-dried four times to afford 20 mg (9% yield) of adenosine 5′-Pα-thio-β,γ-methylenetriphosphate ammonium salt. Final separation of isomers A and B (two diastereoisomers) was carried out by HPLC on a semipreparative reversed-phase column with a TEAA/CH₃CN gradient from 96.6:3.4 to 95:5 over 22 min at a flow rate of 4.5 ml/min: retention time t_{R}(isomer A)=15.8 min, t_{R} (isomer B)=21.4 min.

Data for Isomer A:

¹H NMR (D₂O, 200 MHz) δ 8.62 (s, H-8, 1H), 8.27 (s, H-2, 1H), 6.15 (d, J=6.0 Hz, H-1′, 1H), 5.00 (m, H2′, 1H), 4.60 (m, H-3′, 1H), 4.42 (m, H-4′, 1H), 4.28 (m, H-5′, 2H), 2.28 (t, J=20.0 Hz, CH₂, 2H) ppm; ³¹P NMR (D₂O, 81 MHz) δ 42.9 (d, J=33.5 Hz, Pα-S, 1P), 13.0 (br m, Pγ and Pβ, 2P) ppm; TLC (2:7:11 NH₄OH/H₂O/2-propanol) R_{f}=0.22. The following purity data were obtained on an analytical column: t_{R}=6.48 min (96% purity) using solvent system I with a TEAA/CH₃CN isocratic elution at 95:5 over 10 min at a flow rate of 1 ml/min; t_{R}=2.94 min (95% purity) using solvent system II with a PBS/CH₃CN isocratic elution at 98:2 over 8 min at a flow rate of 1 ml/min.

Data for Isomer B:

¹H NMR (D₂O, 200 MHz) δ 8.67 (s, H-8, 1H), 8.25 (s, H-2, 1H), 6.15 (d, J=6.0 Hz, H-1′, 1H), 5.00 (m, H2′, 1H), 4.59 (m, H-3′, 1H), 4.41 (m, H-4′, 1H), 4.28 (m, H-5′, 2H), 2.31 (t, J=21.0 Hz, CH2, 2H) ppm; ³¹P NMR (D₂O, 81 MHz) δ 43.2 (d, J=32.4 Hz, Pα-S, 1P), 13.2 (br s, Pγ, 1P), 11.9 (br d, Pβ, J=32.4 Hz, 1P) ppm; HRMS ESI (negative) m/z calcd for C₁₁H₁₇N₅O₁₁P₃S⁻ 519.9864. found 519.9853; low-resolution mass spectra were measured for both isomers, and the HRMS spectrum was measured for one of the isomers; TLC (2:7:11 NH₄OH/H₂O/2-propanol) R_{f}=0.22. The following purity data were obtained on an analytical column: t_{R}=9.12 min (97% purity) using solvent system I with a TEAA/CH₃CN isocratic elution at 95:5 over 15 min at a flow rate of 1 ml/min; t_{R}=4.17 min (97% purity) using solvent system II with a PBS/CH₃CN isocratic elution at 98:2 over 10 min at a flow rate of 1 ml/min.

### Example 3

### Synthesis of adenosine 5′-[Pα-thio]-β,γ-(dichloromethylene)triphosphate, APPCCl₂P-α-S

As depicted in Scheme 3, a solution of 2-chloro-4H-1,3,2-benzodioxa phosphorin-4-one (117 mg, 0.58 mmol) in anhydrous DMF (1 ml), was added via syringe to a solution of 2′,3′-orthoformate protected adenosine (100 mg, 0.32 mmol, 3a) and anhydrous pyridine (260 μl) in 1.5 ml of anhydrous DMF at 0° C. under nitrogen. After stirring at RT for 1 h, tributylamine (626 μl) was added, followed by a solution of bis-(tetrabutylammonium)dichloromethylendiphosphonate (127 mg, 0.42 mmol) in anhydrous DMF (1 ml). The reaction mixture was stirred at RT for 2 h, then sulfur (25 mg, 0.77 mmol) was added at 0° C. The solution color changed to orange and then to brown while stirring at RT for 1.5 h. The mixture was dripped to a cold 1 M TEAB solution (10 ml) until pH˜7 was attained. The resulting mixture was stirred at RT for 30 min. During that time the color of the solution changed to yellow. The solution was extracted with ether (2×10 ml). The aqueous phase was freeze-dried twice. The product was then deprotected by addition of 10% HCl until pH 2.3 was obtained and the mixture was stirred for 3 h. Afterwards 24% NH₄OH was added to give pH˜9 and the mixture was stirred for another 45 min and freeze-dried overnight.

The crude residue was separated on a DEAE-Sephadex A25 column (0-0.4 M NH₄HCO₃; total volume 600 ml). The relevant fraction was freeze-dried for four times to afford 43 mg (17% yield) of adenosine 5′-Pα-thio-β,γ-(dichloromethylene)triphosphate ammonium salt. The separation of isomers A and B (two diastereoisomers) was carried out by HPLC on a semipreparative reverse-phase column with a TEAA/CH₃CN gradient from 96.5:3.5 to 95.5:4.5 over 31 min at a flow rate of 4.5 ml/min. Retention time: t_{R}(isomer A)=20.3 min, t_{R}(isomer B)=30.6 min.

Data for Isomer A:

¹H NMR (D₂O, 200 MHz) δ 8.72 (s, H-8, 1H), 8.29 (s, H-2, 1H), 6.16 (d, J=6.0 Hz, H-1′, 1H), (H2′ and H-3′ signals are hidden by the water signal at 4.78), 4.63 (m, H-4′, 1H), 4.35 (m, H-5′, 2H), 3.35 (t, Et₃N, 24H), 2.05 (s, AcOH, H), 1.42 (d, Et₃N, 36H) c; ³¹P NMR (D₂O, 81 MHz) δ 44.0 (d, J=35.4 Hz, Pα-S, 1P), 8.0 (d, J=19.3 Hz, Pγ, 1P), −1.0 (dd, J=19.3 Hz, J=35.4 Hz, Pβ, 1P) ppm; HRMS ESI (negative) m/z: C₁₁H₁₅Cl₂N₅O₁₁P₃S⁻ calculated 587.9084. found 587.9073, low resolution mass was measured for both isomers and HRMS was measured for one of the isomers; TLC (2:7:11 NH₄OH/H₂O/2-propanol) R_{f}=0.35. The following purity data were obtained on an analytical column: t_{R}=9.5 min (99% purity) using solvent system I with a TEAA/CH₃CN isocratic elution 96:4 over 15 min at a flow rate of 1 ml/min; t_{R}=3.35 min (99% purity) using solvent system II with a PBS/CH₃CN isocratic elution 98:2 over 8 min at a flow rate of 1 ml/min.

Data for Isomer B:

¹H NMR (D₂O, 200 MHz) δ 8.65 (s, H-8, 1H), 8.29 (s, H-2, 1H), 6.16 (d, J=6.2 Hz, H-1′, 1H), (H2′ signal is hidden by the water signal at 4.78), 4.64 (m, H-3′, 1H), 4.45 (m, H-4′, 1H), 4.38 (m, H-5′, 2H), 3.15 (t, Et₃N, 24H), 2.05 (s, CH₃CO₂H, 3H), 1.38 (d, Et₃N, 36H) ppm; ³¹P NMR (D₂O, 81 MHz) δ 43.9 (d, J=35.5 Hz, Pα-S, 1P), 8.00 (d, J=19.1 Hz, Pγ, 1P), −0.9 (dd, J=19.1 Hz, J=35.5 Hz, Pβ, 1P) ppm; TLC (2:7:11 NH₄OH/H₂O/2-propanol) R_{f}=0.36. The following purity data were obtained on an analytical column: t_{R}=10.0 min (98% purity) using solvent system I with a TEAA/CH₃CN isocratic elution 95:5 over 15 min at a flow rate of 1 ml/min; t_{R}=4.54 min (98% purity) using solvent system II with a PBS/CH₃CN isocratic elution 98:2 over 10 min at a flow rate of 1 ml/min.

### Example 4

### Titration of Aβ₂₈-Cu⁺ Complex by Various Phosphate-Based Chelators Monitored by ¹H-NMR

¹H-NMR is a sensitive analytical tool that can be utilized, by means of signal width and peak shifts, to observe Aβ-metal-ion coordination, Aβ precipitation, and Aβ resolvation. Since Aβ₄₀ can readily form aggregates at physiological pH (Atwood et al., 1998), we first studied Aβ₂₈, as a more soluble fragment of Aβ₄₀, to evaluate the possibility of application of Cu^{+/2+} chelators for resolvation of Aβ-Cu^{+/2+} oligomers and aggregates. Cu⁺ was selected to induce aggregation due to its diamagnetic properties which enable NMR monitoring of the resolvation process. Moreover, Aβ-Cu⁺ as a reduced form of Aβ-Cu²⁺ aggregates, is of interest since it was suggested to promote initiation of ROS production leading to neuronal apoptosis (Shearer and Szalai, 2008).

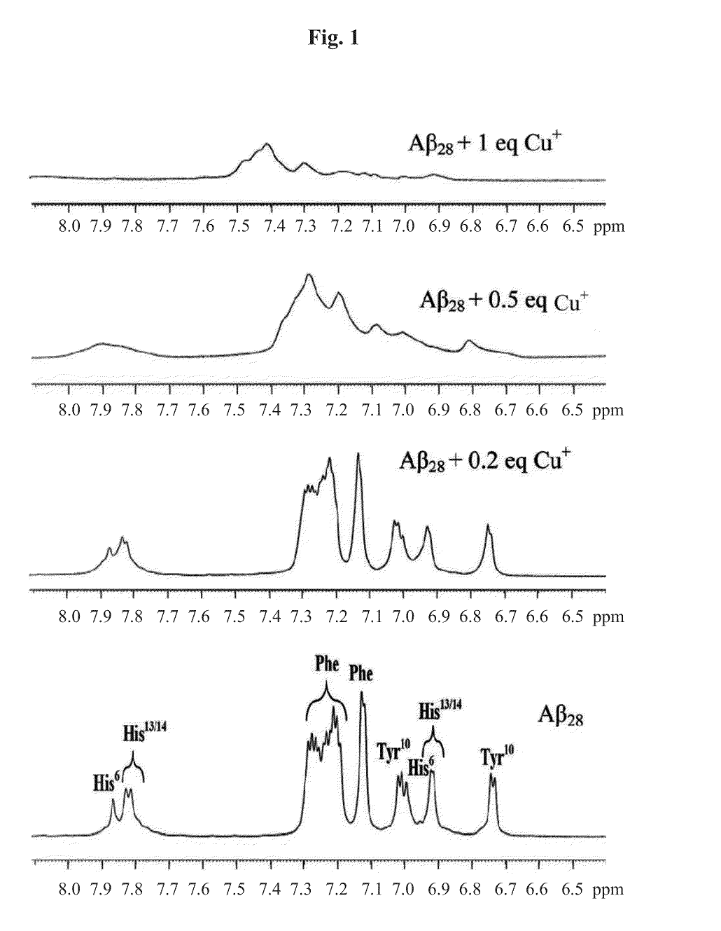

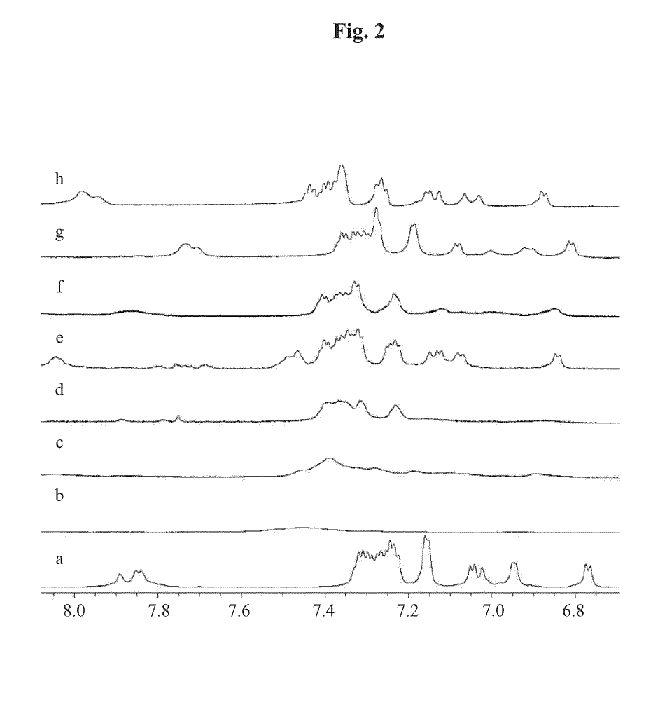

We first conducted ¹H-NMR monitored titrations of Aβ₂₈ in tris-d₁₁ at pD 7 with Cu⁺ to obtain a 1:1 Aβ₂₈-Cu⁺ complex (FIG. 1). Several measures were taken to ensure that Cu⁺ will not oxidize to Cu²⁺: The NMR tube was flushed with argon, oxygen was excluded from the deuterated solvents by bubbling argon, Cu(CH₃CN)₄PF₆ was used as the Cu⁺ source and the concentration of acetonitrile, as stabilizing ligand for Cu⁺, was not less than 15%. The 0.8 mM Aβ₂₈-Cu⁺ complex was titrated by one of the following chelators: thiophosphate, triphosphate, ADP-β-S, GDP-β-S, GTP-γ-S and clioquinol (FIG. 2). By addition of 0.2 eq Cu⁺ the signals of Aβ₂₈ were broadened, and after addition of 1 eq Cu⁺ the peaks were shifted downfield and aromatic Aβ₂₈ signals merged into one very broad signal. To this solution, clioquinol was then added as a standard chelator known for its ability to redissolve metal-ion induced Aβ aggregates (Ritchie et al., 2003). After addition of 6 eq clioquinol the Aβ₂₈-Cu⁺ spectrum slightly sharpened, yet, no signal pattern emerged, and a yellow solid was observed in the NMR tube (FIG. 2c vs. 2b). When 6 eq of triphosphate were added, the Aβ₂₈-Cu⁺ spectrum showed only Phe peaks reappearances without any His signals (FIG. 2d). Thiophosphate (6 eq) was apparently a superior Cu⁺ chelator, the addition of which resulted in a significant sharpening of the Aβ₂₈ spectrum due to the removal of Cu⁺ from Aβ₂₈ (FIG. 2e). Yet, a black solid formed in the NMR tube, possibly a thiophosphate-Cuⁿ⁺ complex. Addition of 6 eq of GDP-β-S resulted in partial recovery of Aβ₂₈ aromatic signals (FIG. 2f). However, unlike the case of thiophosphate, upon addition of GDP-β-S a clear solution was obtained. ADP-β-S proved to be a better chelator (FIG. 2g) and upon addition of 5 eq the Aβ₂₈ spectrum resembled that of pure Aβ₂₈. Furthermore, with ADP-β-S the turbid solution of Aβ₂₈-Cu⁺ turned clear. GTP-γ-S was found to be the best chelator in this series. At only 3.2 eq of GTP-γ-S the Aβ₂₈-Cu⁺ complex spectrum sharpened (FIG. 2h) and looked as that of pure Aβ₂₈ (FIG. 2a). Furthermore, a clear solution was obtained.

Since Cu⁺ is a soft metal-ion it prefers soft ligands such as thiophosphate and nucleoside-5′-phosphorothioate analogues, resulting in a significantly better dissolution of Aβ₂₈-Cu⁺ oligomers as compared to hard ligands such as clioquinol and triphosphate.

GTP-γ-S was found to be the most promising Cu⁺-chelator decomposing Aβ-Cu⁺ oligomers and dissolving Aβ₂₈-Cu⁺ aggregates better than ADP-β-S and GDP-β-S. Namely, a longer phosphate chain binds Cu⁺ ion tighter. Surprisingly, ADP-β-S performed better than GDP-β-S implying the adenine moiety coordinates Cu⁺ better than guanine. Furthermore, we found that ADP-β-S is more stable than GDP-β-S and GTP-γ-S as observed in ³¹P-NMR spectra measured at the end the titration (data not shown).

### Example 5

### Elucidation of the Mode of Chelation of Cu⁺ by Phosphorothioate Compounds Based on ¹H/³¹P-NMR and UV Measurements

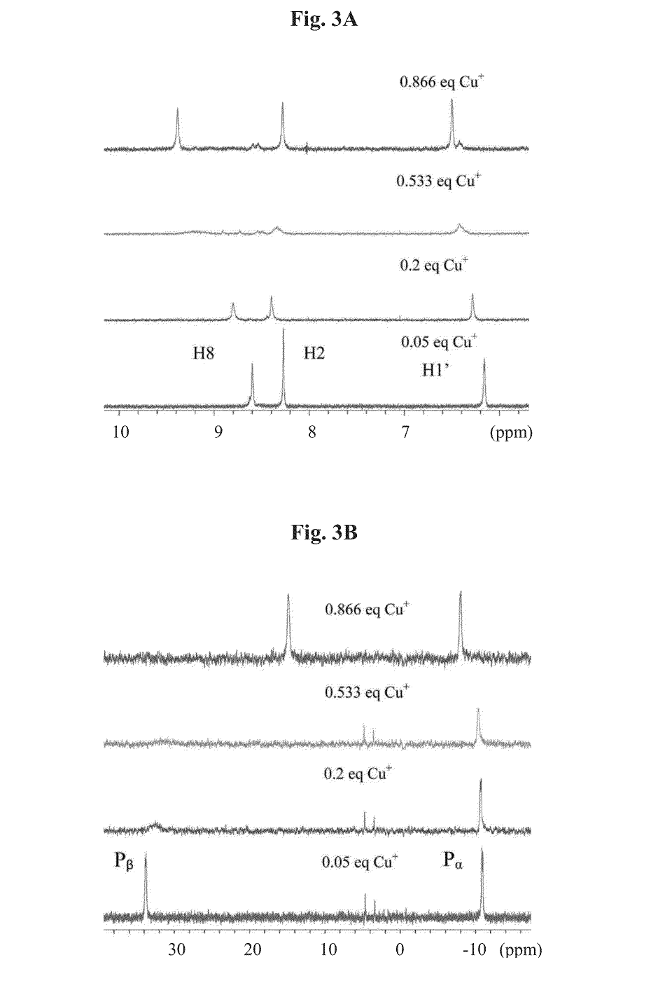

To investigate the mode of Cu⁺ chelation by nucleoside-5′-phosphorothioate analogues, we monitored Cu⁺ titration of ADP-β-S by ¹H/³¹P-NMR (FIG. 3). Changes in the ¹H/³¹P-NMR spectrum were observed upon titration of 9 mM ADP-β-S with up to 0.87 eq of Cu⁺. With the addition of Cu^{+ 1}H-NMR spectra (FIG. 3A) exhibited downfield shift of the adenine H8 signal. Specifically, H8 signal shifted from 8.6 to 9.1 ppm and broadened upon addition of 0.53 eq Cu⁺. However, by the addition of 0.87 eq Cu⁺ H8 signal sharpened and shifted to 9.4 ppm. In ³¹P-NMR spectra we observed an upfield shift of Pβ upon the addition of Cu⁺ (FIG. 3B). P_{β} broadened at 0.53 eq Cu⁺ and after the addition of 0.87 eq Cu⁺, P_{β} reappeared and shifted upfield by 16 ppm. The changes in the ¹H/³¹P-NMR spectrum, broadening and shifting of P_{β} and H8 signals of compound ADP-β-S indicated that the phosphate chain particularly P_{β} phosphorothioate and N7 coordinate with Cu⁺.

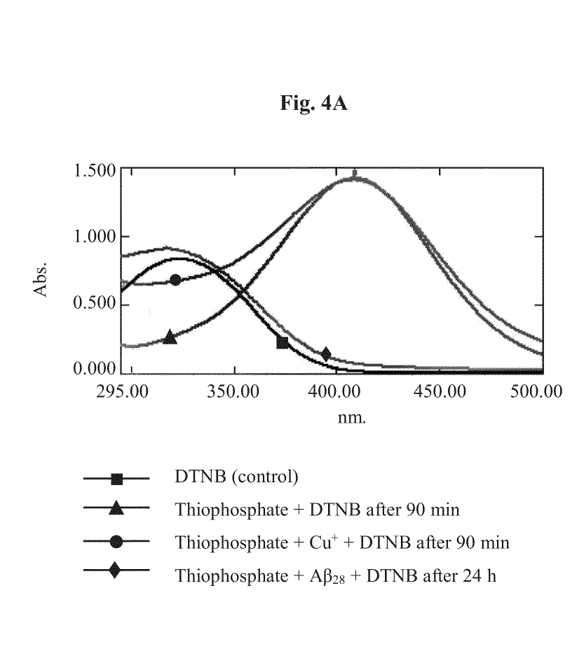

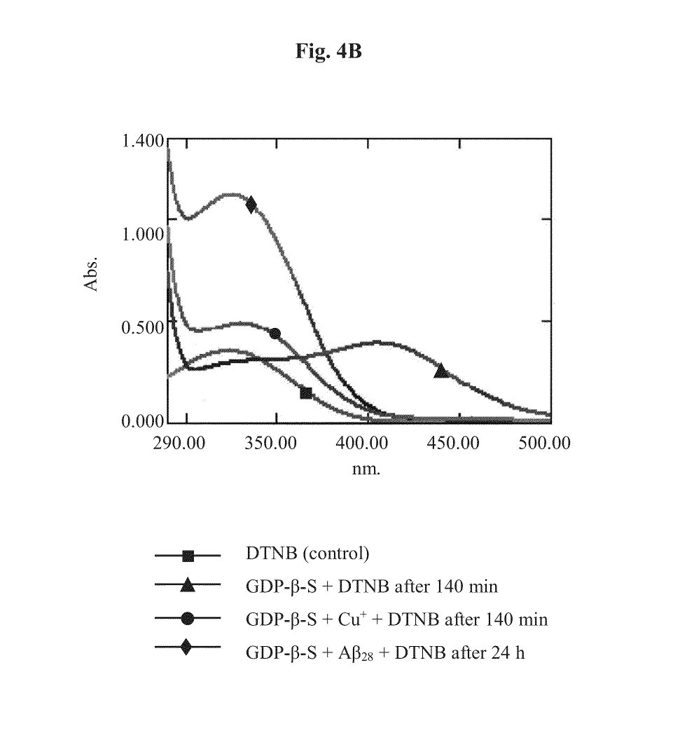

The presence of free thiol in thiophosphate analogues-Cu⁺ solution was determined by Ellman's reagent, 5,5′-dithiobis-(2-nitrobenzoic acid) (DTNB). Ellman's reagent reacts with free thiol to give new disulfide and free 2-nitro-5-thiobenzoate (NTB²⁻) resulting in a yellow mixture absorbing at 412 nm (Goody and Eckstein, 1971). Thiophosphate reacts with Ellman's reagent in the presence or absence of Cu⁺ to give the disulfide (FIG. 4A). However, upon the addition of Aβ₂₈-Cu⁺ to 5 in 1:5 ratio (respectively), no disulfide product was formed even after 24 h (FIG. 4A). BCA, a specific Cu⁺ indicator (Kd 2.16×10⁻¹⁵) (Yatsunyk and Rosenzweig, 2007), was used to quantify Cu⁺ in the sample which was found to be 95% of the starting Cu⁺ amount. Like thiophosphate, GDP-β-S reacts with Ellman's reagent to give the disulfide product; however, upon the addition of Cu⁺ or Aβ₂₈-Cu⁺ and monitoring by UV spectrum, no NTB²⁻ product could be detected (FIG. 4B). Addition of BCA to the sample showed that Cu⁺ remained unchanged. In both cases of thiophosphate and GDP-β-S, a disulfide was formed when reacted with Ellman's reagent. However, unlike disulfide formation with 5-Cu⁺ system, in the case of GDP-β-S—Cu⁺, a disulfide product was not formed. Apparently, the tight complex formed between GDP-β-S and Cu⁺ does not allow the formation of NTB²⁻ upon reaction of GDP-β-S with Ellman's reagent. Interestingly, in both cases of Aβ complexes, Aβ₂₈-Cu⁺-thiophosphate/GDP-β-S, the disulfide product was not observed. This finding possibly indicates that Cu⁺-ion in Aβ₂₈-Cu⁺ complex is still capable of tight interaction with phosphorothioate GDP-β-S. In this way reaction of the thiol with Ellman's reagent is avoided.

### Example 6

### Reduction of Aβ₄₀-M²⁺ Aggregate Size by ATP-γ-S, ADP-β-S, APCPP-γ-S, and GDP-β-S as Monitored by DLS

DLS is used to measure the size of very small particles (0.6 nm to 6 μm) in solution, and it is a well-established technique that had been utilized to measure the hydrodynamic diameter (d_{H}) of monomer or aggregated Aβ₄₀ (Lomakin et al., 1997). However, in the DLS technique a particle is considered as a sphere. Since Aβ is non-spherical by nature, the calculated d_{H} is subject to changes in Aβ conformation.

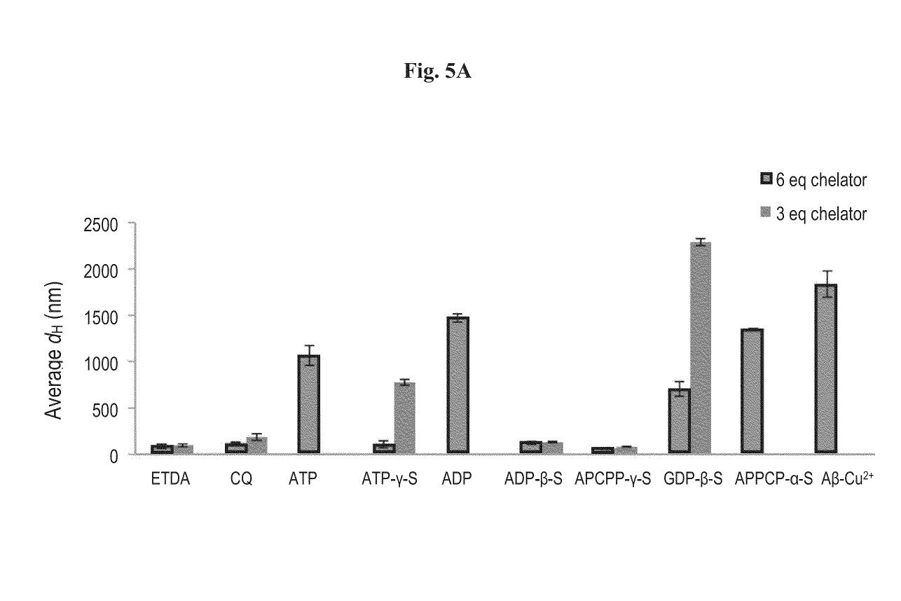

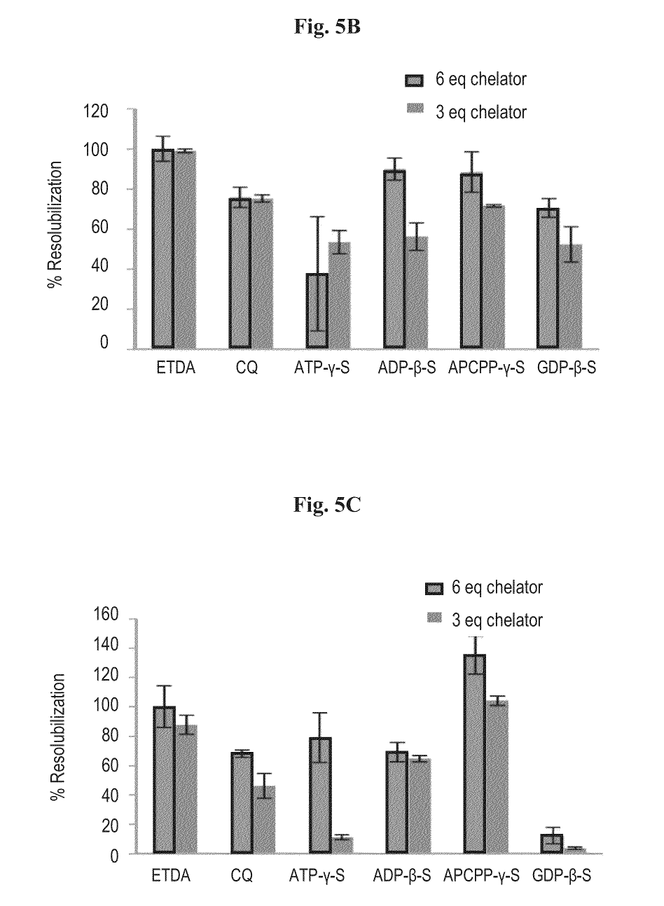

Monomeric Aβ₄₀ was incubated with either Cu²⁺ or Zn²⁺ for 45 min to form a 1:1 peptide metal-ion complex. A precipitate was observed in both cases. We found that for Aβ₄₀-Cu²⁺ the particle size distribution reached a constant value at 1836 nm after 45 min from 1 eq metal-ion addition to Aβ₄₀ monomer. For the Aβ₄₀-Zn²⁺ particles the instrument readings indicated a different size distribution. Next, the chelators ATP, ATP-γ-S, ADP, ADP-β-S, APCPP-γ-S, or GDP-β-S were added and incubated for 30 min, followed by measurement of the d_{H} of the resulting particles (FIG. 5). We evaluated the capacity of the phosphorothioate analogues to dissolve Aβ₄₀-Cu²⁺ or Zn²⁺ aggregates, as compared to EDTA and clioquinol known for their ability to dissolve Aβ-M²⁺ aggregates (Ritchie et al., 2003; Huang et al., 1997). The reduction of d_{H} of Aβ₄₀-Cu²⁺ aggregates by EDTA, clioquinol, or tested compounds was compared to the d_{H} of Aβ-Cu²⁺ aggregate itself (FIG. 5A). In addition, the efficacy of the chelators ATP-γ-S, ADP-β-S, APCPP-γ-S and GDP-β-S to resolubilize Aβ₄₀-Cu²⁺ and Aβ₄₀-Zn²⁺ is described in FIGS. 5B and 5C relative to EDTA and clioquinol. EDTA efficiency in reducing Aβ particle size is considered as 100%.

The d_{H} of Aβ₄₀-Cu²⁺ in the presence of phosphorothioate compounds was smaller compared to that of ADP and ATP (FIG. 5A). Consistent with ¹H-NMR data, ADP-β-S performed 5.4-fold better than GDP-β-S in reducing d_{H} of Aβ₄₀-Cu²⁺ aggregates. Surprisingly, ATP-γ-S did not reduce aggregate d_{H} better than ADP-β-S, possibly because ADP-β-S is more stable in the experimental system. For this reason, it was decided to study more stable derivatives of ATP-γ-S such as APCPP-γ-S and APPCP-α-S. We anticipated that APCPP-γ-S would perform better than APPCP-α-S due to the presence of a terminal thiophosphate moiety. Indeed, compound APCPP-γ-S was highly efficient in reducing d_{H}, more than any of the studied chelators. Although both APCPP-γ-S and ATP-γ-S are nucleoside-5′-triphosphate analogues bearing a terminal thiophosphate moiety, APCPP-γ-S reduced Aβ₄₀-Cu²⁺ particle size to 64 nm vs. 110 nm with compound ATP-γ-S. Clioquinol was less effective than ATP-γ-S, ADP-β-S, and APCPP-γ-S, decreasing the d_{H} of Aβ₄₀-Cu²⁺ to 127 nm. Surprisingly, APCPP-γ-S was more effective even than EDTA in resolubilizing Aβ₄₀-Cu²⁺ (FIG. 5C). ADP-β-S and APCPP-γ-S were equi-efficacious in resolubilizing Aβ₄₀-Zn²⁺ aggregates showing 88-90% of EDTA efficacy (FIG. 5B). Yet, both compounds performed better than clioquinol (ca. 75% of EDTA efficacy) (FIG. 5B, 5C). Interestingly, APCPP-γ-S dissolved Aβ₄₀-Cu²⁺ better than EDTA although the latter is a chelator with high affinity to Cu²⁺ and Zn²⁺ (Log K values for Cu²⁺ and Zn²⁺ EDTA complexes are 18.8 and 16.5, respectively) (Furia, 1980).

### Example 7

### Monitoring Size Reduction of Aβ₄₀-M²⁺ Aggregates by Nucleoside-5′-Phosphorothioate Analogues Using TEM

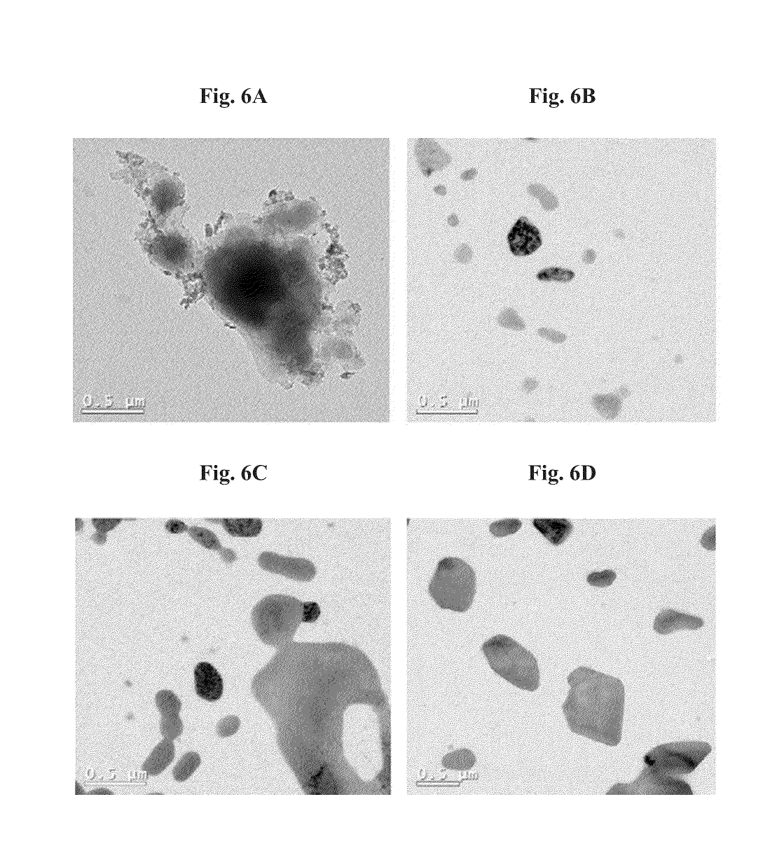

To validate the DLS data indicating the efficiency of APCPP-γ-S, we monitored the dissolution of Aβ₄₀-M²⁺ aggregates in the presence of APCPP-γ-S by TEM. Aβ₄₀-Cu²⁺ and Aβ₄₀-Zn²⁺ 1:1 complexes were incubated for 9 days at RT, resulting in a significant sediment. Aβ₄₀-Cu²⁺ aggregates of 2.5 μm in diameter (FIG. 6A) and Aβ₄₀-Zn²⁺ aggregates of different sizes from 100 nm to 2.5 μm (FIG. 6C) were observed by TEM measurements. APCPP-γ-S (6 eq) was then added and the mixture was incubated for 7 h. Addition of APCPP-γ-S to Aβ₄₀-Cu²⁺ and Aβ₄₀-Zn²⁺ aggregates resulted in a significant reduction in the aggregate size to less than 250 nm for Aβ₄₀-Cu²⁺ and less than 500 nm for Aβ₄₀-Zn²⁺ aggregates, FIGS. 6B and 6D, respectively.

### Example 8

### Re-Solubilization of Aβ₄₀-Cu⁺ Aggregates by APCPP-γ-S Monitored by ¹H-NMR

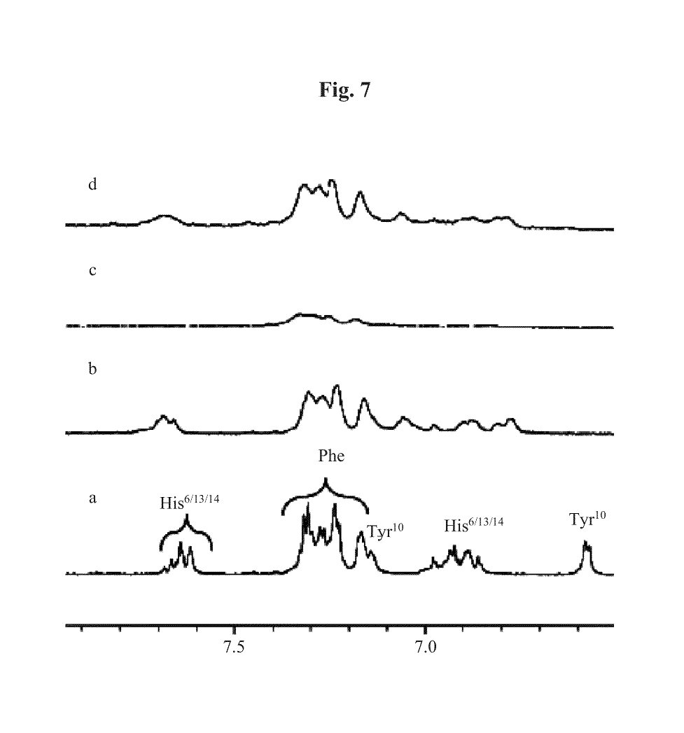

APCPP-γ-S, the most promising chelator identified here, was used also to resolubilize Aβ₄₀-Cu⁺ aggregates. This process was monitored by ¹H-NMR similar to that described above for Aβ₂₈. First, the ¹H-NMR spectrum of 0.25 mM Aβ₄₀ monomer at 278 K, pD 11, was measured (FIG. 7a), after adjustment of Aβ₄₀ solution pD to 7.8 the signals shifted and broadened (FIG. 7b). Upon addition of 1 eq 8.3 mM Cu(CH₃CN)₄PF₆ the signals dramatically further broadened (FIG. 7c). When APCPP-γ-S was added (6 eq) all signals reappeared (FIG. 7d), as seen for Aβ₄₀ at pD 7.8 (FIG. 7b), indicating Cu⁺⁻ coordination by APCPP-γ-S and its removal from Aβ₄₀-Cu⁺ complex. Addition of 9 eq of APCPP-γ-S did not sharpen the spectrum any further.

### Example 9

### Re-Solubilization of Aβ₄₂-Zn²⁺/Cu²⁺ Aggregates by ADP-β-S and APCPP-γ-S Monitored by Turbidity Assay

Aggregation of Aβ-metal ion complexes results in a turbid mixture that increases the light scattering in solution, leading to a higher absorbance at 405 nm (Storr et al., 2009).

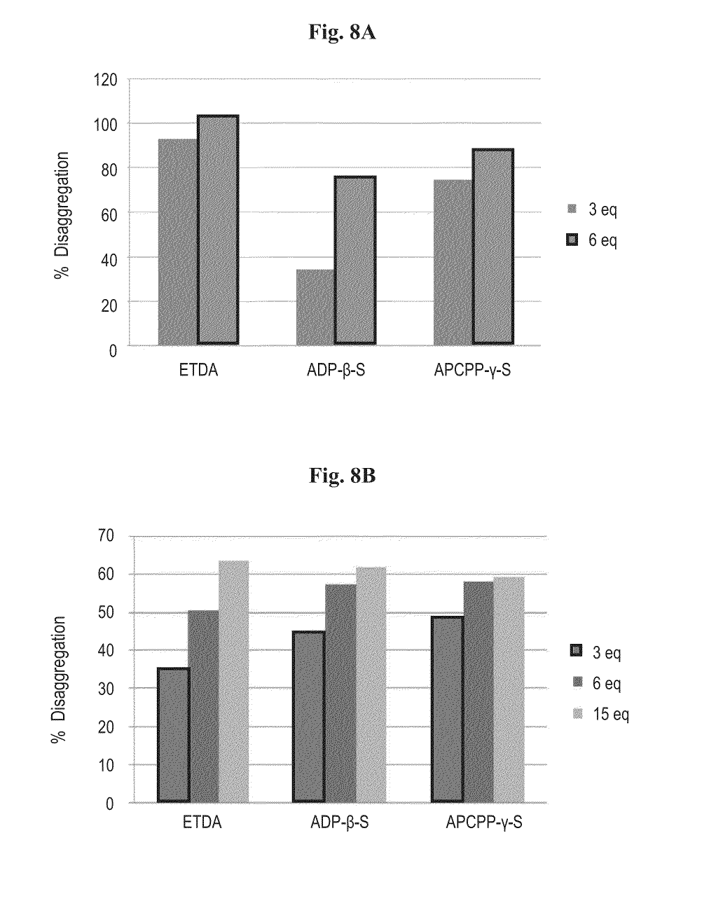

Aggregation of monomeric Aβ₄₂ was achieved by adding Zn(NO₃)₂ to a Aβ₄₂ solution at pH 7.4, and Cu(NO₃)₂ to a Aβ₄₂ solution at pH 6.6. Those 200 μM Aβ₄₂-M²⁺ mixtures, left at RT for 2 h, became turbid, and exhibited an increase of absorbance at 405 nm. The resultant Aβ₄₂ mixtures were assayed for the resolubilization capacity of ADP-β-S and APCPP-γ-S in comparison to EDTA. EDTA was highly effective in decreasing the absorbance of Aβ₄₂-Zn²⁺ mixtures (FIG. 8A). APCPP-γ-S was found to be less effective than EDTA by 21% and 12% at 3 and 6 eq, respectively, and ADP-β-S was less effective than EDTA by 63% and 27% at 3 and 6 eq, respectively (FIG. 8A). However, in the case of Aβ₄₂-Cu²⁺ aggregates, ADP-β-S and APCPP-γ-S were more effective than EDTA at aggregate re-solubilization up to 28% and 12% at 6 and 3 eq, respectively. At 15 eq chelator, EDTA, ADP-β-S and APCPP-γ-S were almost equi-efficacious (FIG. 8B). The turbidity assay confirmed the DLS data that although EDTA has a higher affinity for Zn²⁺ and Cu²⁺ than APCPP-γ-S, the latter was highly effective in decreasing the turbidity of Aβ₄₂-M²⁺ mixtures. Moreover, consistent with the DLS data, APCPP-γ-S was more effective than EDTA in the re-solubilization Aβ₄₂-Cu²⁺ aggregates.

### Example 10

### ESR OH Radical Assay

To study the antioxidant effect of ADP-β-S and APCPP-γ-S, ESR was used to monitor the modulation of .OH formation from H₂O₂ by the Cu⁺ or Fe²⁺ induced Fenton reaction. For this purpose we applied DMPO as a spin trap. The OH radical formed by the reaction of Fe²⁺/Cu⁺ with H₂O₂ is trapped by DMPO, and the DMPO-OH adduct is then detected by ESR. The addition of chelators to Fe²⁺/Cu⁺—H₂O₂ mixture lowers the DMPO-OH signal due to metal-ion chelation and radical scavenging (Richter and Fischer, 2006).

The inhibition of radical production by the chelators ADP-β-S and APCPP-γ-S (expressed in IC₅₀ and IC₉₀ values, Table 1) was compared to the inhibitory effect of common antioxidants including ascorbic acid, GSH and the metal-ion chelator EDTA. Based on our previous reports (Richter and Fischer, 2006; Baruch-Suchodolsky and Fischer, 2008) we expected ADP-β-S and APCPP-γ-S to be more potent inhibitors than ADP and ATP since the formers bear a sulfur substitution at the P_{β}/P_{γ} position. Unlike phosphate, thiophosphate is a soft ligand that binds preferably soft and borderline metal-ions. Indeed, this prediction was found to be true for ADP-β-S and APCPP-γ-S in the Fe²⁺/H₂O₂-system, with IC₅₀ values in the range of 86-100 M (Table 1). Yet, in the Cu⁺/H₂O₂-system, the IC₅₀ values for ADP-β-S and APCPP-γ-S were in the range of 300-400 μM, whereas the parent compounds were mediocre inhibitors of the Cu⁺-induced Fenton reaction (IC₅₀ values of 226 and 183 μM for ADP and ATP). ADP-β-S and APCPP-γ-S were better antioxidants than ascorbic acid and glutathione in the Fe²⁺/H₂O₂-system, the IC₅₀ values of which were 93 and 216 μM, respectively. However, in the Cu⁺/H₂O₂-system GSH was highly efficient with IC₅₀ value of 63 μM, while ascorbic acid was a poor OH radical inhibitor with IC₅₀>500 μM. EDTA, as a better metal-ion chelator, was indeed more efficient than ADP-β-S and APCPP-γ-S in reducing OH radicals production in both systems with IC₅₀ values of 64 and 62 μM, respectively.

TABLE 1
Inhibition of OH radical production in Fenton system by adenine
nucleotides, phosphate, and control antioxidants, as
monitored by ESR

| | IC₅₀ (μM) | | IC₉₀ (μM) | |
|---|---|---|---|---|
| Compound | Cu⁺ | Fe²⁺ | Cu⁺ | Fe²⁺ |
| EDTA | 64 ± 1 | 62 ± 1 | 110 ± 7 | 98 ± 1 |
| Ascorbic acid | N/A | 93 ± 7 | N/A | N/A |
| GSH | 63 ± 5 | W216 ± 4 | N/A | 491 ± 5 |
| ADP | 226 ± 2 | N/A | N/A | N/A |
| ADP-β-S | 408 ± 14 | 100 ± 4 | N/A | 210 ± 1 |
| ATP | 183 ± 1 | N/A | N/A | N/A |
| APCPP-γ-S | 312 ± 23 | 86 ± 3 | N/A | 211 ± 7 |
| Phosphate | N/A | 451 ± 11 | N/A | N/A |

Antioxidant IC₅₀/IC₉₀ values represent the compound's concentration that inhibits 50%/90% of the OH radical amount produced, respectively.
N/A = not available, the minimal amount of radical production exceeds 50% (IC₅₀) or 10% (IC₉₀).

ADP-β-S and APCPP-γ-S were less potent antioxidants in Cu⁺/H₂O₂ system than in Fe²⁺/H₂O₂ system, probably due to oxidation of the phosphorothioate moiety to form a disulfide product in the presence of H₂O₂(Richter and Fischer, 2006), thus concealing the terminal sulfur which might be required for binding Cu⁺ ion. However, the disulfide product apparently binds Fe²⁺-ion better than ADP and ATP, probably by creating a full-coordination sphere as proposed before (Richter and Fischer, 2006). The full coordination sphere provided by the disulfide dimer of ADP-β-S and APCPP-γ-S prevents an electron transfer from Fe²⁺, thus making both ADP-β-S and APCPP-γ-S potent antioxidants.

### Example 11

### Nucleoside 5′-Phosphorothioate Analogues are Potent Antioxidants at PC12 Cells

In this study, nucleoside 5′-phosphorothioate analogues were explored as inhibitors of Fe(II)-induced oxidative stress in PC12 cells used as a neuronal model. Reduction of ROS production in PC12 cells by each one of the tested analogues was measured by DCFH-DA, a radical sensitive indicator. After DCFH-DA was removed, the tested analogue was added to the cells at a final concentration of 0.2-200 μM. Oxidation was initiated by the addition of FeSO₄ (0.16 μM) to the wells. The plates were incubated for 1 h at 37° C., during which the absorbance was read by a Tecan fluorometer at 485/530 nm.

ADP-β-S, GDP-β-S, GTP-γ-S, ATP-γ-S and APCPP-γ-S inhibited ROS formation with IC₅₀ values of 26, 10, 5, 0.18 and 0.16 μM, respectively (values represent mean±S.D of three experiments, P<0.05; data not shown). It should be noted that GDP-β-S and ADP-β-S were 4.5- and 3-fold more stable in PC12 cells than GDP and ADP, respectively. In addition, all the phosphorothioate analogues tested were nontoxic up to 200 μM, and did not harm the basal level of ROS production in cells.

### Example 12

### Nucleoside 5′-Phosphorothioate Analogues are Neuro-Protectants of Primary Neurons Exposed to Oxidative Damages by FeSO₄ or FeSO₄/H₂O₂

Cultured cortical neurons in 96-well plates were treated with different concentrations of FeSO₄, or hydrogen peroxide and FeSO₄, for 24 h at 37° C. Following exposure to various insults, the cells were treated with ATP-γ-S and GDP-β-S as described. Cells were subsequently incubated for a further 18-24 h as indicated before being assessed for viability measures. Neurons were treated with ATP-γ-S and GDP-β-S at three concentrations (25, 100 or 200 Mm) simultaneously with 1.5, 3 or 6 μM FeSO₄ for 24 h. Following 24 h incubation in the presence of both FeSO₄ and ATP-γ-S and GDP-β-S cell viability measures were assessed by XTT assay. All experiments were performed in triplicate.

### Application of FeSO₄ and H₂O₂ to Cultured Neurons Cells

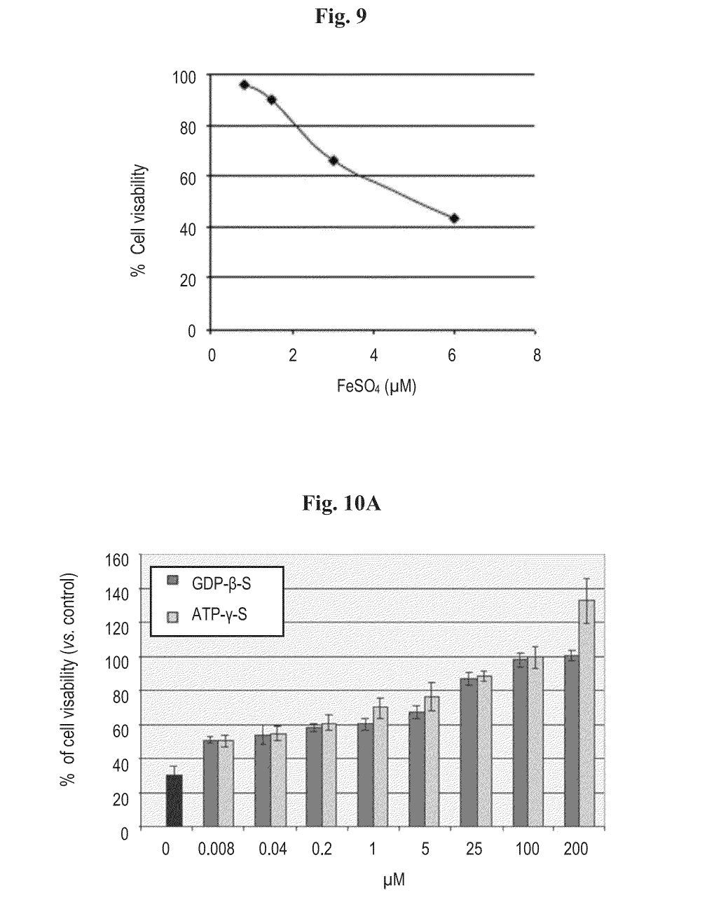

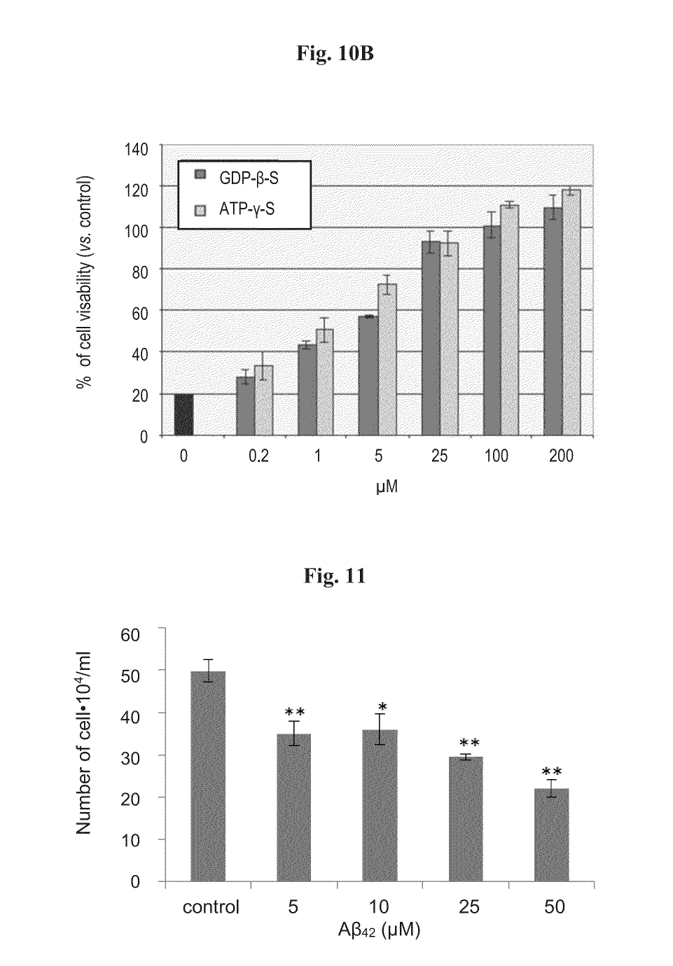

FeSO₄ induced a concentration-dependent decrease in cell viability as assessed by XTT assay and morphological assessment following 24 h of exposure (FIG. 9). The neuroprotective effect of ATP-γ-S and GDP-β-S (due to iron chelation) was evaluated in cortical neurons exposed to FeSO₄ for 24 h (FIG. 10). Co-application of FeSO₄ (3 μM) with ATP-γ-S and GDP-β-S (FIG. 10A) resulted in 100 and 130% protection, respectively. Their IC₅₀ values of ATP-γ-S and GDP-β-S were 0.01 and 0.008 μM, respectively. When the cells were treated with co-application of FeSO₄ (3 μM) and H₂O₂ (100 μM) the IC₅₀ values of ATP-γ-S and GDP-β-S were 1 and 4 μM, respectively (FIG. 10B).

The IC₅₀ values of the nucleoside-5′-phosphorothioate analogues tested were compared to those of the natural nucleotides (Table 2). On the average IC₅₀ values of the synthetic compounds is ˜0.01 μM compared to the natural with IC₅₀ of ˜25 μM, indicating that ATP-γ-S, GTP-γ-S, and ADP-β-S are highly potent neuroprotectants active at the low nanomolar concentrations.

TABLE 2
IC₅₀ values of various nucleoside-5′-phosphorothioate analogues vs.
natural nucleotides

| Nucleotide | IC₅₀ (μM) | Nucleotide | IC₅₀ (μM) |
|---|---|---|---|
| ADP | 19 ± 1.9 | ADP-β-S | 1.2 ± 0.007 |
| GDP | 21 ± 3.1 | GDP-β-S | 0.08 ± 0.003 |
| ATP | 30 ± 2.1 | ATP-γ-S | 0.01 ± 0.008 |
| GTP | 32 ± 2.3 | GTP-γ-S | 0.04 ± 0.01 |
| Trolox | 23 ± 3.1 | | |
| Citrate | 25 ± 3.3 | | |

### Example 13

### APCPP-γ-S Protects Primary Neuron Culture Against Aβ₄₂ Insult

In this study, the neuroprotective activity of APCPP-γ-S in neuronal cells exposed to toxic Aβ₄₂ was evaluated. At first we measured the number of the viable neuronal cells after treatment with Aβ₄₂ (5-50 μM) (FIG. 11). At 50 μM Aβ₄₂, 50% of the neuronal culture remained vital. Next, we measured the protective effect of APCPP-γ-S: primary neurons were treated with APCPP-γ-S (0.04-25 μM) and 50 μM Aβ₄₂ for 48 h.

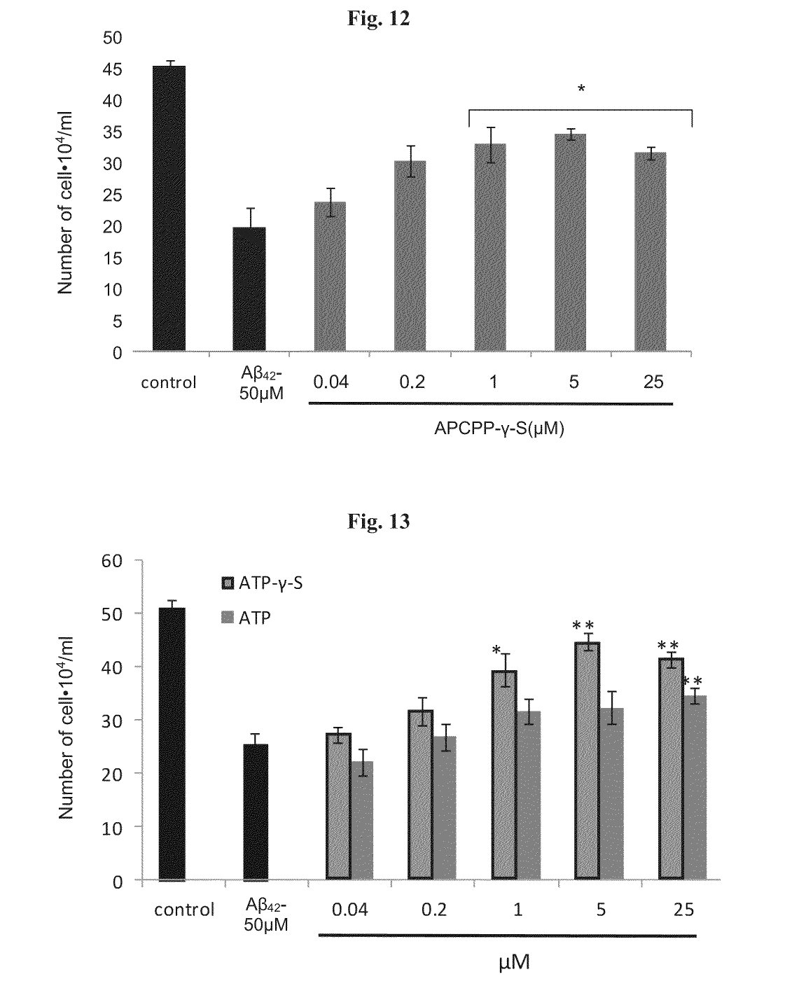

FIG. 12 shows the viability of primary neuronal cells due to the treatment with APCPP-γ-S, in a dose-dependent manner. APCPP-γ-S maintained 50% of neuronal cells at 0.2 μM, while at a similar experiment ATP-γ-S maintained 50% of neuronal cells only at 0.8 μM, and ATP maintained 45% of neuronal cells at 25 μM (FIG. 13).

### Example 14

### Evaluation of APCPP-γ-S, APPCP-α-S and APPCCl₂P-α-S as P2Y_{1/11/2} Receptor Agonists

Antioxidant, antiapoptotic and anti-inflammatory activities were described to be mediated by P2Y receptors. Promising subtypes are the P2Y₁ and P2Y₁₁ receptors (Fujita et al., 2009; Shinozaki et al., 2005). High potency at P2Y receptors enables nucleotides to evoke signals at low concentration which is advantageous from a pharmacological point of view. Modifications at the phosphate groups may change the preference of the receptor for the nucleotide analogues. In this study, the potency of APCPP-γ-S, in which the P_{α}/P_{β} position was modified by a methylene group to improve the chemical and metabolic stability, and one of the non-bridging oxygen atoms at the P, position was replaced by a sulphur atom to improve the antioxidant activity; as well as APPCP-α-S and APPCCl₂P-α-S, in which the P_{β}/P_{γ} position was modified by either a methylene or chloromethylene group, respectively, and one of the non-bridging oxygen atoms at the P_{α} position was replaced by a sulphur atom, to activate P2Y₁, P2Y₂ and P2Y₁₁ receptor was evaluated.

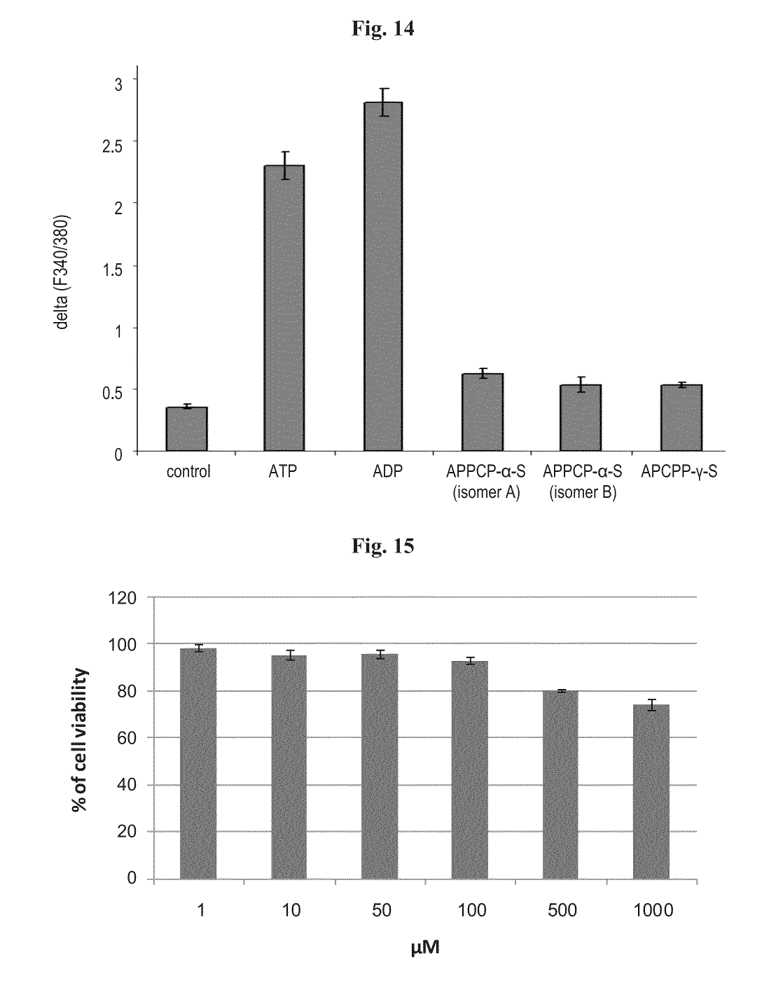

APCPP-γ-S showed very weak potency at the P2Y₁ receptor (no activity up to 10 μM). Interestingly, APCPP-γ-S showed potency at the P2Y₁₁ receptor (EC₅₀=1 μM) being 6.7 times more potent than the natural ligand ATP. APCPP-γ-S was found to be neither agonist nor antagonist at the P2Y₂ receptor (FIG. 14).

APPCP-α-S showed very weak potency at the P2Y₁ receptor (EC₅₀>10 μM). Isomer A was not an agonist of the P2Y₁₁-receptor, and Isomer B was 7-fold more potent than the endogenous P2Y₁₁-receptor ligand, ATP (EC₅₀=6.7 μM).

APPCCl₂P-α-S showed very weak potency at the P2Y₁ receptor (EC₅₀>10 μM). Isomer A was not an agonist of the P2Y₁₁-receptor, and isomer B was 2-fold more active than ATP as a P2Y₁₁-receptor agonist. Isomer A (100 μM) had also no activity as agonist at the P2Y₂ receptor (this analogue did not inhibit the typical response to UTP observed for the P2Y₂R in 1321N1 cells; data not shown).

### Methods

Cell Culture and Transfection.

GFP constructs of human P2Y₂-R, P2Y₁-R and P2Y₁₁-R were stably expressed in 1321N1 astrocytoma cells, which lack endogenous expression of P2X- and P2Y-receptors. The respective cDNA of the receptor gene was cloned into a pEGFPN1 vector and after transfection, using FuGENE 6 Transfection Reagent (Roche Molecular Biochemicals, Mannheim, Germany), cells were selected with 0.5 mg/ml G418 (geneticine; Merck Chemicals, Darmstadt, Germany) and grown in DMEM supplemented with 10% serum (FCS), 100 U/ml penicillin and 100 U/ml streptomycin at 37° C. and 5% CO₂. The expression and cell membrane localization of the respective P2Y receptors was confirmed through the analysis of the GFP fluorescence. The functionality of the expressed GFP-labeled receptor in cells was verified by recording a change of [Ca²⁺]ᵢ after stimulation with the appropriate receptor agonist.

Single Cell Calcium Measurements.

1321N1 astrocytoma cells transfected with the respective plasmid for P2Y-R-GFP expression plated on coverslips (22 mm diameter) and grown to approximately 80% density, were incubated with 2 μM fura 2/AM and 0.02% pluronic acid in Na-HBS buffer (Hepes buffered saline: 145 mM NaCl, 5.4 mM KCl, 1.8 mM CaCl₂, 1 mM MgCl₂, 25 mM glucose, 20 mM Hepes/Tris pH 7.4) for 30 min at 37° C. The cells were superfused (1 ml/min, 37° C.) with different concentrations of nucleotide in Na-HBS buffer. The nucleotide-induced change of [Ca²⁺] was monitored by detecting the fluorescence intensities with excitations at 340 nm and 380 nm. Only GFP-labeled cells were analyzed. Microsoft Excel (Microsoft Corp., Redmond, Wash., USA) and SigmaPlot (SPSS Inc., Chicago, Ill., USA) were used to derive the concentration-response curves and EC₅₀ values from the average response amplitudes obtained in at least three independent experiments (Ecke et al., 2006; Ecke et al., 2008). Only cells with a clear GFP-signal and with the typical calcium response kinetics upon agonist pulse application were included in the data analysis. The nucleotide induced change of [Ca²⁺]ᵢ was monitored by detecting the respective emission intensity of fura 2/AM at 510 nm with 340 nm and 380 nm excitations (Ubl et al., 1998). The average maximal amplitude of the responses and the respective standard errors were calculated from ratio of the fura 2/AM. The GFP-tagged P2Y receptors are suitable for pharmacological and physiological studies, as previously reported (Tulapurkar et al., 2004; Tulapurkar et al., 2006; Zylberg et al., 2007).

### Example 15

### APCPP-γ-S is Metabolically Stable

In order to use APCPP-γ-S as a neuroprotectant agent and test its activity in an animal model, we first evaluated the metabolic stability of APCPP-γ-S in several tissues such as liver, brain and blood. In this assay we took samples from mice brain, liver and blood. The tissue samples were homogenated by sonication and split to 0.5 ml aliquots. Each sample was mixed with 0.1 mg of APCPP-γ-S and incubated at 37° C. for 10, 30, 90 and 180 min. After incubation, the samples were collected and extracted with chloroform at 1:1 v/v ratio. The aqueous layer was removed and freeze-dried. Samples were loaded onto an activated Starta X-AW weak anion exchange cartridge, washed with H₂O (1 ml) and eluted with MeOH:H₂O (1:1, 1 ml) followed by NH₄OH:MeOH:H₂O (2:25:73, 1 ml), and then freeze-dried. The resulting residue was analyzed by HPLC. All chromatographic analyses were performed at 30° C. using SUPELCOSIL™ LC-18-S HPLC Column (5 μm particle size, L×I.D. 25 cm×2.1 mm), flow rate 0.2 ml/min under isocratic elution conditions with the following buffer composition: [50 mM potassium phosphate, 100 mM triethylamine, 0.1 mM MgCl₂, pH 6.5 (adjusted with phosphoric acid)]:Acetonitrile (98.5:1.5). Each analysis cycle was set to 30 minutes. The chromatographic flow was monitored at 260 nm and integrated using EZChrom Elite Software.

As shown from the chromatograms (data not shown), APCPP-γ-S could be detected even after 180 min in brain, liver and blood.

### Example 16

### APCPP-γ-S is of Limited Toxicity at PC12 Cells Up to 1000 μM

In this study, the toxicity of APCPP-γ-S, at 1-1000 μM, at PC12 cells was tested by MTT assay. FIG. 15 shows that APCPP-γ-S was not toxic to PC12 cells after 24 h of incubation up to 100 μM. At 1000 μM, 75% of the cells were still viable.

### Example 17

### Pharmacokinetics and BBB Permeation of APCPP-γ-S

As shown in Example 14, APCPP-γ-S is stable in human blood serum as well as in brain, liver and blood from mice. In this study, we evaluated APCPP-γ-S blood-brain barrier (BBB) permeability and pharmacokinetics in F1 (C57B×S29) mice. APCPP-γ-S was injected intravenously (IV) at 40 mg/Kg into four mice (each mouse was injected with 1.5 mg of APCPP-γ-S). The mice were sacrificed after 30 minutes (2 of the 4) or 90 minutes (thr other 2 of the 4), and samples from brain and blood were taken to analysis. Each sample was collected into 0.5 ml saline, sonicated and extracted with chloroform, applied onto an anion exchange cartridge, and freeze-dried. The resulting residue was analyzed by HPLC. We detected the presence of 67.5 and 64% of the injected amount of APCPP-γ-S in the blood samples after 30 and 90 minutes, respectively. In the brain sample we obtained ca. 2% permeability (data not shown).

### Example 18

### Synthesis of Nucleoside 5′-Phosphorothioate Prodrugs

In order to achieve oral bioavailability and intracellular delivery of the nucleotide analogues exemplified herein, e.g., APCPP-γ-S and ADP-β-S, various prodrug strategies have been explored, mainly focusing on the partial masking of these analogues' negatively charged backbone by bioreversible, lipophilic groups, that provide the prodrug permeability through different membrane tissues. The prodrug is then decomposed, either spontaneously or enzymatically, in the brain, and releases the biologically active compound.

The BBB forms an interface between the circulating blood and the brain, and possesses various carrier-mediated transport systems for small molecules such as glucose and amino acids to support and protect CNS function (Ohtsuki and Terasaki, 2007). Hence, the development of drugs that structurally mimic substrates of influx transport is an effective strategy to increase BBB permeability.

Synthesis of ADP-β-S Analogue Based on Conjugation with D-Glucose

It was assumed that by coupling these nucleoside-5′-phosphorothioate analogues identified herein as promising neuroprotectants, i.e., ADP-β-S and APCPP-γ-S, with D-glucose, which is the main energy source for the brain and transported by GLUT1, it will be possible to increase the permeability of these nucleoside-5′-phosphorothioate analogues through glucose transporters. As depicted in Scheme 4, D-glucopyranoside-1-α-thiophosphate, synthesized according to the literature (Singh et al., 1988), was coupled with AMP in the presence of CDI and ZnCl₂ to yield 1-D-glucosyl-β-ADP-β-S.

Synthesis of APCPP-γ-S Analogue Based on Conjugation with D-Glucose (Scheme 5)

### Adenosine-2′,3′-Methylidene

Adenosine (2 g, 7.49 mmol) was dissolved in dry DMF (13.1 ml) under N₂ atmosphere. TsOH (2.85 g, 15 mmol) was added to the reaction flask as solid. Then, trimethylorthoformate (41 ml, 37.5 mmol) was introduced into the flask. After 3 days of reaction a DOWEX (free base form) was added with cooling of the reaction flask in ice water bath. The mixture was stirred for 2 h and filtered. The filtrate was evaporated to get yellow oil. Three extractions with CHCl₃ (70 ml)/NaHCO₃ (70 ml) were performed followed by extraction with brine (70 ml). An organic phase was dried with Na₂SO₄, filtered and evaporated. Crystallization from acetone with cooling in ice water bath was performed to get 1.15 g (49.6%) of the compound. ¹H-NMR (CDCl₃, 200 MHz): 8.50 (s, 1H), 8.14 (s, 1H), 7.2 (bs, 2H), 6.25 (s, 1H), 6.15 (d, 1H), 4.65 (m, 1H), 4.45 (m, 1H), 4.40 (m, 1H), 4.02 (m, 2H), 3.47 (s, 1H), 3.41 (s, 1H) ppm, ¹³C-NMR (CDCl₃, 50 MHz): 155.7, 152.2, 150.0, 139.8, 125, 120, 97.5, 86.5, 74.5, 73.0, 70.1, 52.1 ppm, MS (ESI, negative mode): 309.

### Adenosine-2′,3′-Methylidene-5′-Tosyl

Adenosine-2′,3′-methylidene (523 mg, 1.7 mmol) was dissolved in dry DCM (30 ml) under N₂ atmosphere at RT. 4-dimethylaminopyridine (837 mg, 6.9 mmol) was dissolved in dry DCM (3 ml) and introduced dropwise into the reaction flask. Tosyl chloride (795 mg, 4 mmol) after crystallization and drying was dissolved in dry DCM (5 ml) and dropped into the reaction flask. After 3 h the reaction was monitored by TLC (DCM:MeOH 9:1) and almost complete consumption of the reagent was observed. Three extractions with saturated sodium bicarbonate (40 ml) were performed. The organic phase was evaporated to get 380 mg (47%) of the product. ¹H-NMR (CDCl₃, 200 MHz): 8.50 (s, 1H), 8.14 (s, 1H), 7.56 (dd, J=6 Hz, 2 Hz, 2H), 7.2 (bs, 2H), 7.12 (dd, J=6 Hz, 2 Hz, 2H), 6.25 (s, 1H), 6.15 (d, 1H), 4.65 (m, 1H), 4.45 (m, 1H), 4.40 (m, 1H), 4.02 (m, 2H), 3.47 (s, 1H), 3.41 (s, 1H), 2.31 (s, 3H) ppm, ¹³C-NMR (CDCl₃, 50 MHz): 155.7, 152.2, 150.0, 144.3, 140.7, 139.8, 130.5, 128.3, 125, 120, 97.5, 86.5, 74.5, 73.0, 70.1, 52.1, 24 ppm, MS (ESI, negative mode): 463.

### HPLC Purification for Adenosine-2′,3′-Methylidene-5′-Tosyl

The purification was performed on silica column of Biotage apparatus with ethanol (strong solvent) and dichloromethane (weak solvent) elution. The gradient of the strong solvent was: 0%-3%-3 CV; 0%-8%-10 CV; 8%-10%-3 CV; 10%-10%-3 CV; 10%-90%-1 CV; and 90%-90%-2 CV. The first of a two peaks was collected and evaporated to get mixture of two stereoisomers of the product.

### Adenosine-5′-Methylene-Diphosphate

Adenosine-2′,3′-methylidene-5′-tosyl (285 mg, 0.6 mmol) was dissolved in dry DMF (1 ml) in a two-neck flask under N₂ atmosphere. Methylene-diphosphate (265 mg, 1.5 mmol) tetrabutylammonium salt (obtained by passing methylene-diphosphonic acid through Sephadex CM resin (tetrabutylammonium form)) was evaporated for 3 times with dry DMF (1 ml each time). After 2 days, the reaction was monitored by ³¹P-NMR and signals of both methylene-diphosphate and the product were observed (16.4 ppm (s), 14.8 (d, J=19.4 Hz), 18.9 (d, J=19.4 Hz)). The solvent was evaporated and the product was deprotected by 10% HCl (0.5 ml, pH 2.3) treatment with stirring for 2.5 h. 10% Ammonium hydroxide solution (0.3 ml, pH 9) was added and the solution was stirred for 45 min. The solution was freeze-dried and the residue was applied to DEAE-Sephadex anion-exchange column for LC purification with ammonium bicarbonate buffer (pH 7.5, 600 ml+600 ml of water) with 0.0 M-0.3 M gradient. Final purification was achieved by reverse phase HPLC separation with TEAA (0.0 M-0.4 M) eluent to get 58 mg (21%) of the product. ¹H-NMR (D₂O, 200 MHz): 8.50 (s, 1H), 8.14 (s, 1H), 7.2 (bs, 2H), 6.25 (s, 1H), 6.15 (d, 1H), 4.65 (m, 1H), 4.45 (m, 1H), 4.40 (m, 1H), 4.15 (m, 2H), 3.47 (s, 1H), 3.41 (s, 1H) ppm, ¹³C-NMR (D₂O, 50 MHz): 155.0, 153.0, 150.0, 139.1, 123, 120, 97.5, 86.5, 74.5, 72.0, 70.1, 52.1, 41 ppm, ³¹P-NMR (D₂O, 83 MHz): 16.4 (s), 14.8 (d, J=19.4 Hz), 18.9 (d, J=19.4 Hz) ppm, MS (ESI, negative mode): 464.

The D-glucose was added to a HEPES buffer solution (20 mmol, 4.76 g) containing sucrose phosphorylase (60 U), sodium thiophosphate (0.30 mmol, 54 mg), sucrose (342.3 mg, 1.0 mmol), and magnesium sulfate (0.30 mmol, 73.9 mg) at 25° C. The solution was stirred for 24 h at RT. The reaction progress was monitored by TLC chromatography (H₂O:isopropanol:NH₄OH 6:12:2) with the Ellman's reagent development which stained phosphorothioate compounds. Two spots were observed on the TLC plate: R_{f}=0.355 (product), R_{f}=0.067 (reagent). At the end of the reaction (determined by TLC) the solution was filtered through Amicon PM-30 filter and freeze dried. The product was separated by LC (DEAE-Sephadex anion-exchange column, from 0.0 M to 0.3 M of TEAB buffer) monitored by TLC and Ellman's reagent staining. The fraction containing product was freeze dried to get 126.5 mg of product (46% yield). ¹H-NMR (D₂O, 200 MHz): 5.6 (d, 1H, J=3.4 Hz), 3.7-3.85 (m, 3H), 3.35-3.45 (m, 2H), 3.5-3.6 (m, 1H) ppm. ¹³C-NMR (D₂O, 50 MHz): 92, 73, 72, 71, 69, 60 ppm. ³¹P-NMR (D₂O, 83 MHz) δ 43.9 ppm (s). MS (ESI, negative mode): 275.

### Glucose-1-α-ATP-α,β-Methylene-γ-S

Adenosine-methylenediphosphate and glucose-1-α-thiophosphate were converted to corresponding trioctylammonium and tributylammonium salts by passing them through Sephadex CM resin (trioctylammonium and tributylammonium form, respectively). Adenosine-methylenediphosphate (114 mg, 0.093 mmol) was evaporated for 3 times with dry DMF (1 ml each time) and introduced into a two-neck dry flask under N₂ atmosphere in dry DMF (1 ml). CDI (1,1′-carbonyldiimidazole, 151 mg, 0.93 mmol) was introduced into the reaction flask as solid. After 6 h the reaction was monitored by TLC (water:isopropanol:NH₄OH 7:11:2) indicating almost complete consumption of the adenosine-methylenediphosphate. Dry methanol (38 μl, 0.93 mmol) was added and after 8 min ZnCl₂ (59 mg, 0.43 mmol) was introduced into reaction flask. After 2 min glucose-1α-thiophosphate (227 mg, 0.279 mmol) was added. TLC (water:isopropanol:NH₄OH 7:11:2) was performed after 21 h showing no further progress of the reaction. After 22 h the reaction was stopped by addition of EDTA (192 mg, 0.516 mmol), water (5 ml and TEAB (1 M, 0.5 ml) till pH 7.5 was attained. The mixture was freeze-dried, and the residue was purified by LC on DEAE-Sephadex anion-exchange column eluting with 0.0 M-0.3 M gradient ammonium bicarbonate buffer (pH 7.5, 600 ml of each). Fractions containing product were freeze-dried several times to get 4.1 mg of the product (4.5% yield). The final purification was performed by HPLC separation with TEAA buffer and acetonitrile with gradient of 3%-20% of acetonitrile. Two diastereomers were collected separately at the 8% of acetonitrile. ¹H-NMR (D₂O, 200 MHz): 8.71 (s), 8.47 (s), 8.23 (s), 7.99 (d, J=8.5 Hz), 7.74 (d, 8.5 Hz), 7.63 (s), 7.43-7.28 (m), 6.93-6.86 (m), 6.07 (d, J=5.6 Hz), 4.3-4.47 (m), 4.1-4.16 (m), 3.6-3.75 (m) ppm. ¹³C-NMR (D₂O, 50 MHz): 155.0, 153.0, 150.0, 139.1, 120.3, 103.5, 97.5, 86.5, 78.1, 74.5, 72.0, 71.9, 71.5, 70.1, 69.3, 60.5, 20.3 ppm, ³¹P-NMR (D₂O, 83 MHz): 18.35 (m), 19.38 (m), 34.25 (m) ppm.

### Example 19

### Synthesis of Uridine/Adenosine-5′-Tetrathiobisphosphonate and Di-Uridine/Di-Adenosine 5′,5″-Tetrathiobisphosphonate

As depicted in Scheme 6, in order to synthesize uridine/adenosine-5′-tetrathio bisphosphonate we applied methylene-bis(1,3,2-dithiaphospholane-2-sulfide), 6b, prepared from bis-methylene(phosphonicdichloride) that was treated with 1,2-ethanedithiol and 10 mol % AlCl₃ in CHBr₃. As has been shown, primary alcohols can successfully react with 6b to yield O,O′-diester-methylenediphosphonotetrathioate analogues (Amir et al., 2013). Compounds 6c-u and 6c-a were obtained from 6a-u and 6a-a, respectively, in a one-pot reaction. First, 2′,3′-methoxymethylidene uridine, 6a-u (or 2′,3′-methoxymethylidene adenosine, 6a-a) was treated with 6b in the presence of molecular sieves in DCM for 24 h, and then, a mixture of 1 eq. of DBU in DCM was added dropwise over 1 h period. The reaction progress was monitored by ³¹P-NMR, the formation of doublets at 100.1 and 90.4 ppm indicated the formation of intermediate 6c-u (or 6c-a), also indicated by the cloudy reaction mixture turning immediately clear. Without isolating the product, 3-hydroxypropionitrile (6 eq.) and DBU (1 eq.) were added to the reaction flask at 45° C. ³¹P-NMR indicated the formation of 6d-u (doublets at 104.8 and 104.5 ppm). The work-up of the reaction included filtration of the molecular sieves and evaporation of the solvent. This one-pot synthesis is highly moisture sensitive, thus the use of molecular sieves in this process is necessary. Steps a and b were performed in a one-pot reaction because attempts to isolate products 6c-u and 6c-a on a reverse phase column resulted in hydrolytic ring opening of the thiophospholane ring, as indicated by MS analysis and ³¹P-NMR (peaks at 105 and 67 ppm).

Products 6d-u and 6d-a were separated on a silica gel column applying CHCl₃:MeOH (85:15) eluent. Further purification was performed on medium pressure chromatography on a reverse phase column using IM TEAA (pH=7):CH₃CN (78:22) eluent. Products 6d-u and 6d-a were obtained in 37% and 28% yield, respectively. Products 6e-u and 6e-a were obtained after treatment with tBuO⁻Na⁺ in THF resulting in β-elimination. The formed acrylonitrile was scavenged with ethanethiol while products 5e-u and 5e-a precipitated from the reaction mixture together with EtSNa salt. The solvent was removed by decantation and the solid residue was dissolved in water and titrated with 10% HCl followed by the addition of 40% NH₄OH, for the removal of the methoxymethylidene protecting group. The residue was subjected to Sephadex DEAE ion-exchange chromatography to yield the desired products UPCP-α,α′,β,β′-tetra-S and APCP-α,α′,β,β′-tetra-S in 45% and 55% yield, respectively (from 6d-u and 6d-a, respectively).

As depicted in Scheme 7, this new synthetic route was expanded further to obtain the corresponding di-uridine/di-adenosine 5′,5″-tetrathiobisphosphonate. Intermediates 7c-u and 7c-a were synthesized from reaction of 7b with 7a-u and 7a-a, respectively. Due to the high moisture sensitivity of this reaction step, compounds 7a-u (or 7a-a) and 7b were stirred together with molecular sieves in dry acetonitrile overnight. Then, DBU (2.1 eq.) was added at 60° C., and the completion of the reaction was monitored by ³¹P-NMR. After 2 h, the desired intermediate 7c-u (or 7c-a) was obtained. The work-up and the removal of the protecting group were performed as mentioned above for uridine/adenosine 5′-tetrathiobisphosphonate. Final purification of the di-uridine/di-adenosine 5′,5″-tetrathio bisphosphonate obtained was carried out by HPLC, on a reverse-phase column, applying IM TEAA (pH=7):CH₃CN eluent. Di-uridine 5′,5″-tetrathio bisphosphonate and di-adenosine 5′,5″-tetrathio bisphosphonate were obtained in 30% and 36% yield, respectively.

### UDP-β-Tetrathiobisphosphonate Tris-Ammonium Salt, UPCP-α,α′,β,β′-Tetra-S

To a two necked round bottom flask containing molecular sieves, 6b (150 mg, 0.462 mmol), 6a-u (264.65 mg, 0.924 mmol) and DCM (4.5 ml) were added. The mixture was stirred overnight under nitrogen atmosphere, and then, a mixture of DBU (0.462 mmol, 0.07 ml) in DCM (4.5 ml) was added dropwise, over a period of 1 h. ³¹P-NMR showed the formation of 6c-u (doublets at 100.10 and 90.45 ppm). 3-Hydroxypropionitrile (2.772 mmol, 0.19 ml) and DBU (0.462 mmol, 0.07 ml) were then added. The reaction mixture was stirred under nitrogen at 45° C. for 30 min. ³¹P-NMR showed the formation of 6d-u (doublets at 104.8 and 104.5 ppm). The mixture was filtered and the molecular sieves were washed with DCM. After evaporation of the solvent, 6d-u was separated on silica column using CHCl₃:MeOH (85:15). This fraction was further purified on a reverse phase column using TEAA IM (pH=7):CH₃CN (78:22) eluent, to give 6d-u in 37% yield (130 mg). ¹H NMR (acetone-d₆; 600 MHz): δ 8.42 (d; J=7.8 Hz; 1H), 6.06-6.07 (m; 2H), 5.77 (d; J=7.8 Hz; 1H), 5.48 (dd; J=6.0 Hz; J=1.8 Hz; 1H), 5.19 (dd; J=6.0 Hz; J 3.6=Hz; 1H), 4.43-4.46 (m; 2H), 4.30-4.32 (m; 3H), 3.51 (td; J=13.2 Hz; J=1.8 Hz; 2H), 3.27 (s; 3H), 2.88 (td; J=7.5 Hz; J=1.8 Hz) ppm. ³¹P NMR (acetone-d₆; 81 MHz): δ 105.28 (d; J=25.8 Hz; P_{α}), 104.18 (d; J=25.8 Hz; P_{β}) ppm. ¹³C NMR (acetone-d₆; 151 MHz): δ 163.6, 151.5, 143.6, 119.2, 118.1, 103.3, 90.9, 85.4 (d; J=9.8 Hz), 84.9, 81.8, 64.6 (d; J=6.6 Hz), 62.7 (t; J=60.6 Hz), 60.1 (d; J=6.5 Hz), 50.9, 19.9 (d; J=8.6 Hz) ppm. HR MALDI (negative): Calcd for C₁₅H₂₀N₃O₈P₂S₄, 559.960. found, 559.957.

Product 6d-u (130 mg, 0.17 mmol) was dissolved in THF (3 ml) and ethylmercaptane (3 ml). Then potassium tert-butoxide (57.3 mg, 0.51 mmol) was added in portions. After 2 h, ³¹P-NMR indicated the presence of only the starting material in the solution and a mixture of the starting material and the product in the precipitate obtained in the reaction. The solution was treated with an additional portion of potassium tert-butoxide (57.3 mg, 0.51 mmol). The solid residue was dissolved again in THF (3 ml) and ethylmercaptane (3 ml). After 1.5 h, ³¹P-NMR showed no starting material in the solution and the desired product, 6e-u, was observed in the precipitate. The solvent was removed by decantation and the solid was dissolved in water and freeze-dried. Product 6e-u was dissolved in water and then titrated with 10% HCl until pH=2.4 was achieved. The mixture was stirred at RT for 3 h. Then 40% NH₄OH was added until pH=9 and stirred for 45 min. The mixture was freeze-dried. The residue (100 mg, yield: 90%) was subjected to ion-exchange chromatography (on a DEAE Sephadex column, swollen overnight in 1 M NaHCO₃ at 4° C.). The product was eluted applying a gradient of 0-0.5 M (800 ml each) of ammonium bicarbonate solution, pH 7.6, to obtain UPCP-α,α′,β,β′-tetra-S in 45% yield (40 mg). ¹H NMR (D₂O; 600 MHz): δ 8.14 (d; J=7.8 Hz; 1H), 5.95 (d; J=8.4 Hz; 1H), 5.91 (d; J=5.4 Hz; 1H), 4.50 (dd; J=4.8 Hz; J=4.2; 1H), 4.44 (t; J=5.4 Hz; 1H), 4.25-4.26 (m; 3H), 3.43 (t, J=13.2 Hz, PCH₂P, 2H) ppm. ³¹P NMR (D₂O; 81 MHz): δ 106.26 (d; J=22.5 Hz; P_{α}), 78.06 (d; J=22.5 Hz; P_{β}) ppm. ¹³C NMR (D₂O; 151 MHz): δ 166.1, 151.8, 142.4, 102.5, 87.9, 83.5, 73.7, 69.9, 62.9, 60.3 (t; J=56.3 Hz) ppm. HR MALDI (negative): Calcd for C₁₀H₁₅N₂O₇P₂S₄, 464.923. found, 464.920.

### ADP-α,β-Tetrathiobisphosphonate Tris-Ammonium Salt, APCP-α,α′,β,β′-Tetra-S

Product APCP-α,α′,β,β′-tetra-S was prepared according to the above procedure for the preparation of UPCP-α,α′,β,β′-tetra-S. Compound 6c-a was obtained from 6a-a (285.80 mg, 0.924 mmol) and 5b (150 mg, 0.462 mmol) in 28% yield (100 mg). ¹H NMR (acetone-d₆ 600 MHz): δ 9.09 (s; 1H), 9.03 (s; 1H), 8.19 (s; 1H), 8.18 (s; 1H), 6.46 (d; J=3.6 Hz), 6.22 (d; J=3.6 Hz), 6.16 (s; 1H), 5.98 (s; 1H), 5.45-5.63 (m; 3H), 5.41-5.42 (m; 1H), 4.59-4.62 (m; 1H), 4.53-4.58 (m; 1H), 4.26-4.30 (m; 4H), 3.55-3.62 (m; 4H), 3.42 (s; 3H), 3.27 (s; 3H), 2.87-2.89 (m; 4H) ppm. ³¹P NMR (acetone-d₆; 81 MHz): δ 105.35 (d; J=25.9 Hz; P_{α}), 104.02 (d; J=25.9 Hz; P_{β}) ppm. ¹³C NMR (acetone-d₆; 151 MHz): δ 156.5, 156.4, 153.5, 150.3, 141.6, 141.5, 119.6, 119.5, 119.1, 117.8, 90.4, 89.9, 87.1 (d; J=9.9 Hz), 86.1, 85.5 (d; J=9.7 Hz), 85.4, 82.8, 82.4, 65.0 (d; J=6.9 Hz), 64.5 (d; J=6.8 Hz), 61.5-62.3 (m; PCP), 59.7-59.9 (m; CH₂—O), 52.4, 50.8 ppm. HR MALDI (negative): Calcd for C₁₆H₂₁N₆O₆P₂S₄, 582.987. found, 582.987.

After LC separation, APCP-α,α′,β,β′-tetra-S was obtained in 55% yield (38 mg). ¹H NMR (D₂O; 600 MHz): δ 8.74 (s; 1H), 8.27 (s; 1H), 6.12 (d; J=5.4 Hz; 1H), 4.90 (t; J=5.4 Hz; 1H), 4.68 (dd; J=4.2 Hz; J=4.8 Hz; 1H), 4.28-4.44 (m; 3H), 3.47 (t, J=13.2 Hz, PCH₂P, 2H) ppm. ³¹P NMR (D₂O; 81 MHz): δ 104.99 (d; J=21.1 Hz; P_{α}), 90.59 (d; J=21.1 Hz; P_{β}) ppm. ¹³C NMR (D₂O; 150 MHz): δ 154.4, 151.1, 148.8, 140.9, 118.5, 87.1, 84.0, 74.3, 70.5, 62.9, 57.8 (t; J=59.0 Hz) ppm. HR MALDI (negative): Calcd for C₁H₁₆N₅O₅P₂S₄, 487.950. found, 487.952.

### Di-Uridine-5′,5″-Diphosphate-α,β-Methylene-α,β-Tetra-Thiophosphate-Bis-Triethylammonium Salt, UPCPU-α,α′,β,β′-Tetra-S

To a two necked round bottom flask containing molecular sieves, 7a-u (281.60 mg, 0.984 mmol), 7b (80 mg, 0.246 mmol) and dry acetonitrile (7 ml) were added. The mixture was stirred overnight under nitrogen atmosphere. Then DBU (0.520 mmol, 0.08 ml) was added and the mixture was stirred at 60° C. for 2 h. ³¹P-NMR showed the formation of the desired product 7c-u (singlet at 105.1 ppm). The mixture was filtered and the molecular sieves were washed with CHCl₃. After evaporation of the solvent, 7c-u was separated on a silica-gel column using CHCl₃:MeOH (90:10).

Most of product 7c-u (73 mg) was dissolved in water and then titrated with 10% HCl until pH=2.4 was achieved. The mixture was stirred at RT for 3 h. Then 40% NH₄OH was added until pH=9 and stirred for 45 min. The mixture was freeze-dried. The residue was purified on a reverse phase column using 1M TEAA (pH=7):CH₃CN (92:8) eluent, to give UPCPU-α,α′,β,β′-tetra-S in 30% yield (65 mg). Final purification was carried out by HPLC, using a semipreparative reverse-phase column, applying an isocratic TEAA/CH₃CN 92:8 in 15 min (4 ml/min): t_{R} 9.36 min.

³¹P NMR (D₂O; 81 MHz): δ 104.02 (s; 2P) ppm. ¹H NMR (D₂O; 600 MHz): δ 8.15 (d; J=8.4 Hz; 1H), 5.95-5.98 (m; 2 Hz), 4.49 (dd; J=4.8 Hz; J=4.2 Hz; 1H), 4.44 (t; J=4.8 Hz; 1H), 4.28-4.32 (m; 3H), 3.51 (t; J=13.8 Hz; PCH₂P; 2H), 3.19 (q; J=7.2 Hz; 5H), 1.27 (t; J=7.2 Hz; 8H) ppm. ¹³C NMR (D₂O; 150 MHz): δ 166.4, 151.9, 142.2, 102.4, 88.4, 83.2 (t; J=4.8 Hz), 73.9, 69.8, 62.3, 57.4 (t; J=65 Hz), 46.5, 8.1 ppm.

### Di-Adenosine-5′,5″-Diphosphate-α,β-Methylene-α,β-Tetra-Thiophosphate-Bis-Triethylammonium Salt, APCPA-α,α′,β,β′-Tetra-S

Compound APCPA-α,α′,β,β′-tetra-S was prepared according to the same procedure as for UPCPU-α,α′,β,β′-tetra-S. Compound 7c-a was obtained from 7a-a (371 mg, 1.19 mmol) and 7b (100 mg, 0.308 mmol). Compound APCPA-α,α′,β,β′-tetra-S was obtained after the removal of the methoxymethylidene protecting group from the intermediate 7c-a and purified on a reverse phase column using 1M TEAA (pH=7):CH₃CN (93:7) eluent, to give APCPA-α,α′,β,β′-tetra-S in 36% yield (100 mg). Final purification was carried out by HPLC, using a semipreparative reverse-phase column, applying an isocratic elution with TEAA/CH₃CN 90:10 in 15 min (4 ml/min): t_{R}8.35 min.

¹H NMR (D₂O; 600 MHz): δ 8.50 (s; 1H), 8.06 (s: 1H), 6.01 (d; J=5.4 Hz), 4.61 (t; J=4.2 Hz; 1H), 4.43-4.47 (m; 1H), 4.28-4.35 (m; 2H), 3.59 (t; J=14.4 Hz; PCH₂P; 2H), 3.19 (q; J=7.2 Hz; 5H), 1.27 (t; J=7.2 Hz; 8H) ppm. ³¹P NMR (D₂O; 81 MHz): δ 104.44 (s; 2P) ppm. ¹³C NMR (D₂O; 150 MHz): δ 154.7, 152.5, 148.2, 139.6, 117.6, 87.2, 84.0 (t; J=5.1 Hz), 75.6, 70.7, 62.2, 57.9 (t; J=68.7 Hz), 46.5, 8.1 ppm.

### Example 20

### Evaluation of Chemical Properties of Adenosine-5′-Tetrathio Bisphosphonate and Di-Adenosine 5′,5″-Tetrathiobisphosphonate

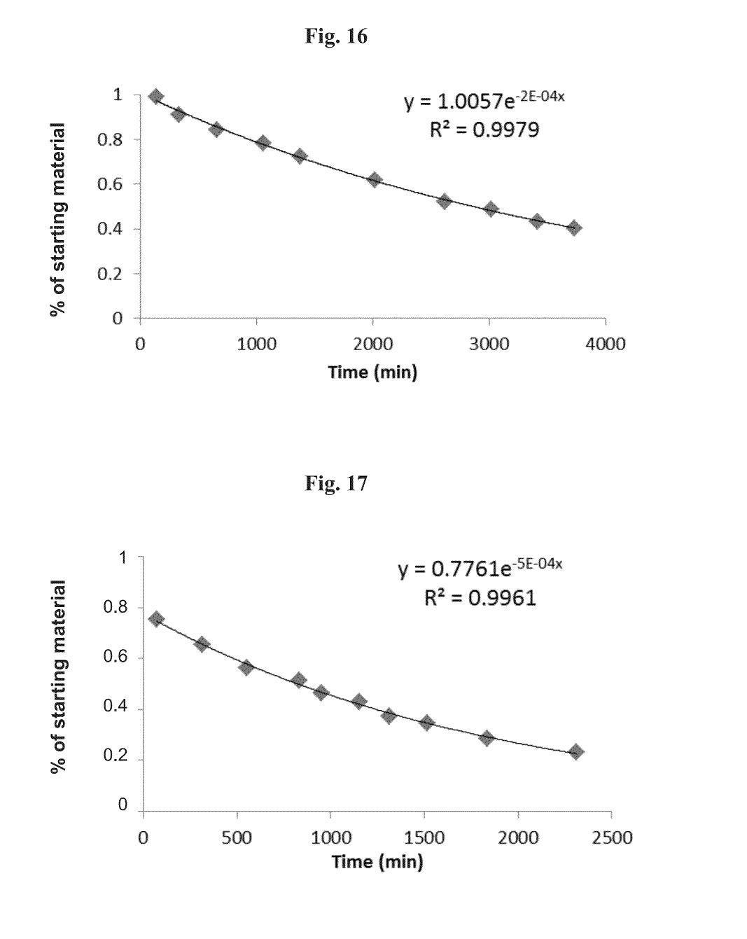

In order to study and evaluate the chemical stability of adenosine-5′-tetrathio bisphosphonate and di-adenosine 5′,5″-tetrathiobisphosphonate to basic and acidic condition, as well as to air-oxidation, kinetic measurements were performed by monitoring the changes in the percentage of these amalogues, using ³¹P-NMR (FIGS. 16-18).

The evaluation of the stability of adenosine-5′-tetrathio bisphosphonate by ³¹P-NMR was first conducted at pD=1.5 for four days. In the course of the experiment, new signals emerged in ³¹P-NMR spectra at 104.4, 92.3, 89.2, 86.1, 67.8 ppm and the percentage of starting material was obtained from the ratio of integration between the starting material and the total peaks in the spectrum. As shown in FIG. 16, adenosine-5′-tetrathio bisphosphonate was relatively stable under these conditions with calculated half-life of 44 h. Mass spectrum (ESI-QTOF negative) analysis of freeze-dried adenosine-5′-tetrathio bisphosphonate after four days at pD 1.5 revealed the fragmentation products shown in Scheme 8.

In the mass spectrum, we observed the signal that can be correlated to 8b, m/z 488, and the fragmentations of the hydrolysis products. The combination of mass analysis with ³¹P-NMR data for 8b subjected to acidic media for 4 days reveals that the signals at 104.4 and 67.8 ppm are correlated to the asymmetric hydrolysis product 8a m/z 472. Moreover, the mass spectroscopic analysis and the ³¹P-NMR shift at 92.3 ppm revealed the presence of MDPT, 239 m/z. In addition, the shift at 86.1 ppm in ³¹P-NMR can be correlated with the formation of oxidized MDPT product 8c (237 m/z). The singlet at 89.2 ppm can be correlated with compound 8d that formed by an intramolecular nucleophilic attack and the loss of water. Moreover, four-membered ring heterocyclic compounds such as 8d were reported before, and the typical ³¹P-NMR signal at ˜90 ppm we found here for 8d is in accordance with previous findings (Toyota et al., 1993).

Next, we studied the stability of adenosine-5′-tetrathio bisphosphonate under basic conditions, pD=11. We found that compound 8b is highly stable under these conditions. After two weeks, the ³¹P-NMR spectrum was identical to the starting material, without any indication of decomposition. We associate this with the repulsion between the negative charges of the tetrathio-bisphosphonate moiety in adenosine-5′-tetrathio bisphosphonate and OH⁻ ions. In addition, intramolecular nucleophilic attack and formation of disulfide bond are less likely to occur under these conditions.

Furthermore, we tested the stability of adenosine-5′-tetrathio bisphosphonate under air-oxidizing conditions, by performing ³¹P-NMR measurements in an open rotating NMR tube. The half-life of adenosine-5′-tetrathio bisphosphonate under these conditions was 14 h. The MS and ³¹P-NMR analysis of the freeze-dried sample of adenosine-5′-tetrathio bisphosphonate after 3 days indicated the formation of an intramolecularly oxidized product. The new asymmetric centers that formed after the oxidation of adenosine-5′-tetrathio bisphosphonate resulted in complex multiplets signals in the ³¹P-NMR spectrum (˜105 and ˜65 ppm).

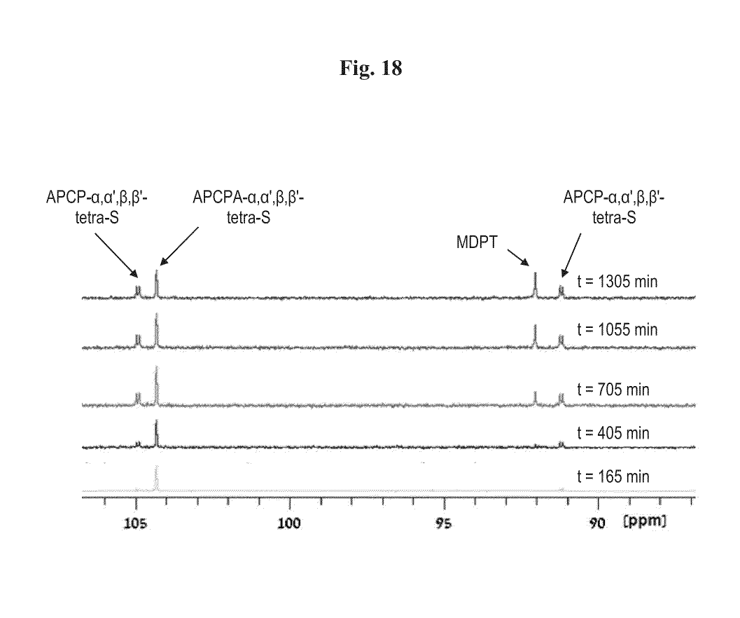

Di-adenosine 5′,5″-tetrathiobisphosphonate exhibited half-life of 9 h under pD 1.5. The combination of mass analysis with ³¹P-NMR data for di-adenosine 5′,5″-tetrathiobisphosphonate subjected to acidic media for 2 days revealed that di-adenosine 5′,5″-tetrathiobisphosphonate undergoes decomposition, first to mono-nucleotide, adenosine 5′-tetrathiobisphosphonate. ³¹P-NMR showed two indicative doublets at 105 and 91 ppm (FIG. 18) that correspond to the chemical shifts of adenosine 5′-tetrathiobisphosphonate. Then, MDPT, was formed as indicated by a singlet at 92 ppm. These observations are supported by MS analysis of the freeze-dried sample.

Di-adenosine 5′,5″-tetrathiobisphosphonate was highly stable under air-oxidizing conditions in an open NMR tube for 3 days. No change in di-adenosine 5′,5″-tetrathiobisphosphonate was observed. The dinucleotide scaffold increased the resistance to oxidation and formation of a disulfide bond.

At pD=1, adenosine 5′-tetrathiobisphosphonate was completely stable even after two weeks. The stability of di-adenosine 5′,5″-tetrathiobisphosphonate was identical to that of adenosine 5′-tetrathiobisphosphonate under these conditions. Moreover, ¹H-NMR indicated that the bridging methylene hydrogen atoms are exchangeable, since the methylene typical triplet signal had broadened and the integration of this peak decreased. The exchange of the hydrogen atoms with deuterium at pD 11 implies on the acidity of the phosphonate methylene group.

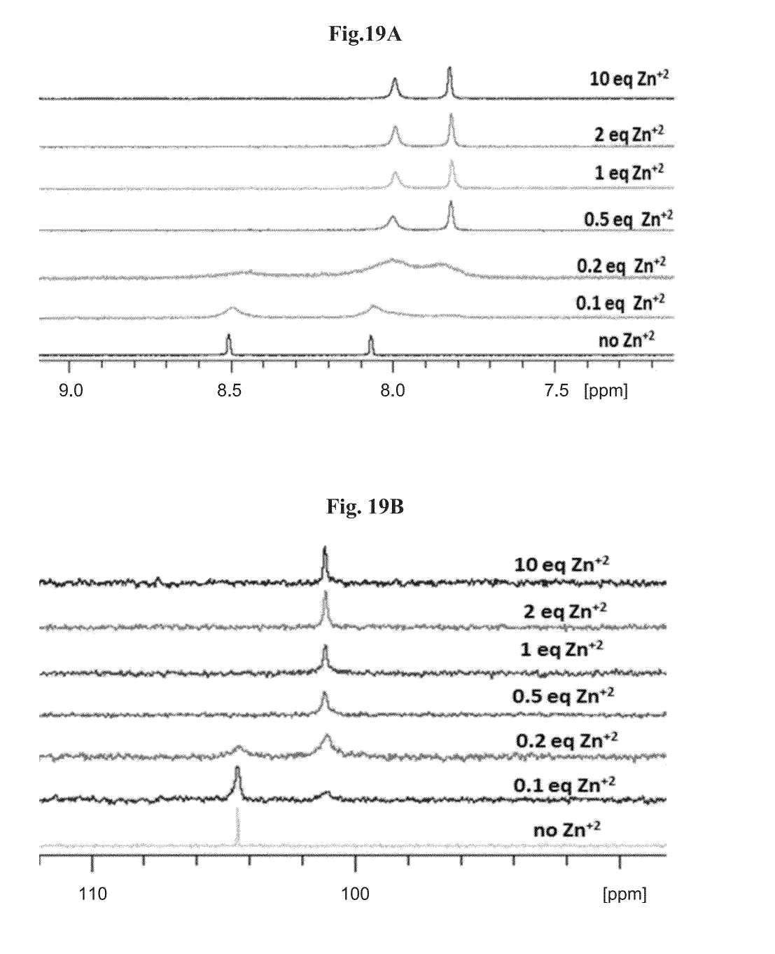

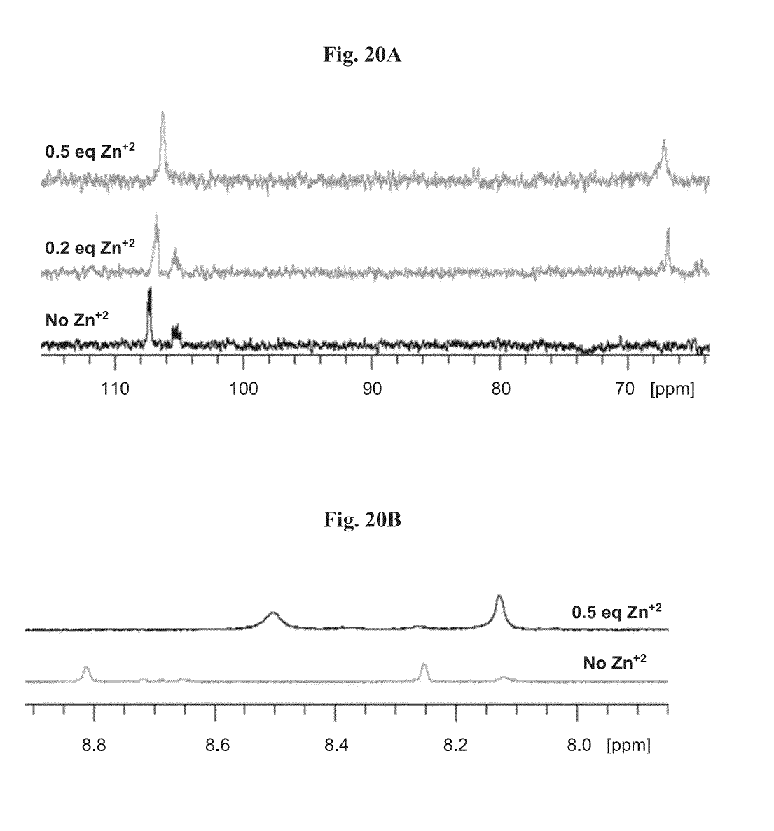

In order to determine the Zn²⁺-coordination to adenosine 5′-tetrathiobis phosphonate and di-adenosine 5′,5″-tetrathiobisphosphonate, we preformed Zn²⁺-titration monitored by ¹H- and ³¹P-NMR spectroscopy (FIGS. 19-20). The shift of NMR signals as well as their line-broadening indicates Zn²⁺-coordination to several atoms in both analogues tested. Solutions of the analogues tested were titrated by 0.1-10 eq Zn²⁺ and monitored by ¹H- and ³¹P-NMR at 400 and 160 MHz, respectively. Relatively low nucleotide concentrations were used (3-5 mM) to avoid inter-molecular base stacking. The titration was performed with 0.2-0.35 M Zn²⁺ solutions in D₂O at pD 7.4 and 300K. Chemical shifts (δ_{H}, δₚ) were measured at different Zn²⁺ concentrations.

Addition of 0.1 eq Zn²⁺ to di-adenosine 5′,5″-tetrathiobisphosphonate caused line-broadening and an upfield shift. Line-broadening is a result of dynamic equilibrium between the free ligand, e.g. said analogue, and the Zn²⁺-ligand complex. The singlet at 103 ppm corresponds to the free ligand, and the emerging singlet at 100.5 ppm corresponds to the Zn²⁺-ligand complex. After addition of 0.5 eq Zn²⁺ only one singlet at 100.5 ppm is observed. Line-sharpening indicates that there is no free ligand, i.e., all molecules of the analogue are engaged in Zn²⁺ complex. These results are consistent with common tetrahedral geometry of zinc complexes, in which two ligands of di-adenosine 5′,5″-tetrathiobisphosphonate form a complex with one zinc ion. The Δδ value of di-adenosine 5′,5″-tetrathiobisphosphonate due to metal-ion binding is 3 ppm. The addition of up to 10 eq of Zn²⁺ resulted in no change in ³¹P-NMR spectrum.

Data of ¹H-NMR monitored Zn²⁺-titrations for di-adenosine 5′,5″-tetrathiobis phosphonate are presented in FIG. 20A and the results show a similar trend. After addition of only 0.1/0.2 eq of Zn²⁺, shift of signals and line-broadening was evident for di-adenosine 5′,5″-tetrathiobisphosphonate. H8 was shifted upfield by 0.5 ppm while H2 was shifted by 0.2 ppm. The shifts of H8 imply that N7 is a coordination site of Zn²⁺. Whereas the upfield shifts of H2 in the presence of zinc ions possibly result from stacking interactions (Stern et al., 2010). Addition of Zn²⁺ to a solution of adenosine 5′-tetrathiobisphosphonate resulted in upfield shifts, both in ³¹P— and ¹H-NMR monitored titrations. Upon the addition of 0.2 eq Zn²⁺ there are two types of species, free adenosine 5′-tetrathiobisphosphonate and Zn²⁺-adenosine 5′-tetrathiobisphosphonate complex. When 0.5 eq Zn²⁺ were added all adenosine 5′-tetrathiobisphosphonate molecules were engaged in zinc complex and P_{β} signal shifted 38 ppm upfield. The tremendous shift of ˜40 ppm indicates that the sulfur modification has a high affinity to Zn²⁺ and the terminal phosphate is involved in metal-ion binding, as was shown for the terminal thiophosphate analogues ATP-γ-S, ADP-β-S and GDP-β-S. Data of ¹H-NMR monitored Zn²⁺-titrations for adenosine 5′-tetrathiobisphosphonate show line-broadening and upfield shifts of H2 and H8. The upfield shift of H8 by 0.3 ppm implies that N7 is a coordination site of Zn²⁺, as also found for di-adenosine 5′,5″-tetrathiobisphosphonate.

### Example 21

### Adenosine-5′-Tetrathiobisphosphonate and Di-Adenosine-5′,5″-Tetrathiobisphosphonate are Highly Stable to Hydrolysis by Human Ectonucleotidases Compared to ADP-β-S and GDP-β-S, and Aβ₂A, Respectively

Since NPP hydrolyzes the Pα-β bond, adenosine-5′-tetrathiobisphosphonate was compared with ADP-β-S to examine the effect of methylene group and extra thiophosphonate groups on the stability and inhibition of NPP1. In addition, the effect of the nucleobase was examined by comparing ADP-β-S with GDP-β-S.

NPP1 activity was measured at pH 8.5. Human NPP1 preparation was added to the incubation buffer at 37° C., and the reaction was started by the addition of a particular nucleotide analogue, and terminated after 1-2 h by addition of perchloric acid. The nucleotide degradation products were separated and quantified by HPLC, and the concentrations of reactants and products were determined from the relative areas for their absorbance maxima peaks. The acid used to terminate the enzymatic reaction can cause partial degradation of the nucleotide analogues. Therefore, the percentage of degradation for each analogue due to the acidic treatment was assessed in the absence of enzyme, and this value was subtracted from the percentage of analogue degradation in the presence of enzyme. Human NPP1 hydrolyzed all analogues tested to NMP and either pyrophosphate or inorganic phosphate, wherein the identity of the degradation products was determined by comparing their retention times to those of controls.

TABLE 3
Hydrolysis of adenosine-5′-tetrathiobisphosphonate, di-adenosine-5′,5″-
tetrathiobisphosphonate, ADP-β-S and GDP-β-S by human ectonucleotidases

| | Relative hydrolysis (% ± SD of ADP or AP₂A hydrolysis) | | | |
|---|---|---|---|---|
| Human | adenosine-5′-tetra | di-adenosine-5′,5″-tetra | | |
| ectonucleotidase | thiobisphosphonate | thiobisphosphonate | ADP-β-S | GDP-β-S |
| NPP1 | ND | ND | ND | 98 ± 1 |
| NPP3 | ND | ND | 25 ± 2.8 | 32 ± 2.1 |
| NTPDase1 | 1 ± 0.2 | ND | ND | — |
| NTPDase2 | 1 ± 0.2 | ND | ND | — |
| NTPDase3 | ND | 2.0 ± 0.1 | 2 ± 0.1 | — |
| NTPDase8 | 7 ± 1 | 3.0 ± 0.1 | 14 ± 0.1 | — |

As shown in Table 3, over 2 h period, adenosine-5′-tetrathiobisphosphonate, di-adenosine-5′,5″-tetrathiobisphosphonate and ADP-β-S were not metabolized at all by NPP1, but the latter was significantly hydrolyzed by NPP3 at 25%. However, GDP-β-S was significantly metabolized by both NPP1 and 3 at 21% and 32%, respectively, indicating higher rate of hydrolysis for the guanine nucleotide compared with that for the adenine nucleotide. Adenosine-5′-tetrathiobisphosphonate, di-adenosine-5′,5″-tetrathio bisphosphonate, and ADP-β-S modified with dithiophosphonate and thiophosphate groups were both stable towards NPP1, NPP3 and NTPDases hydrolysis. The terminal thiophosphate group in ADP-β-S and the methylene group in adenosine-5′-tetrathiobisphosphonate and di-adenosine-5′,5″-tetrathiobisphosphonate conferred stability to NPP and NTPDase hydrolysis, since the latter bond, P_{α-β}, is cleaved in ADP analogue.

In the next study, adenosine-5′-tetrathiobisphosphonate, di-adenosine-5′,5″-tetrathiobisphosphonate and ADP-β-S were evaluated as inhibitors of ectonucleotidases, using the protocol described in Experimental.

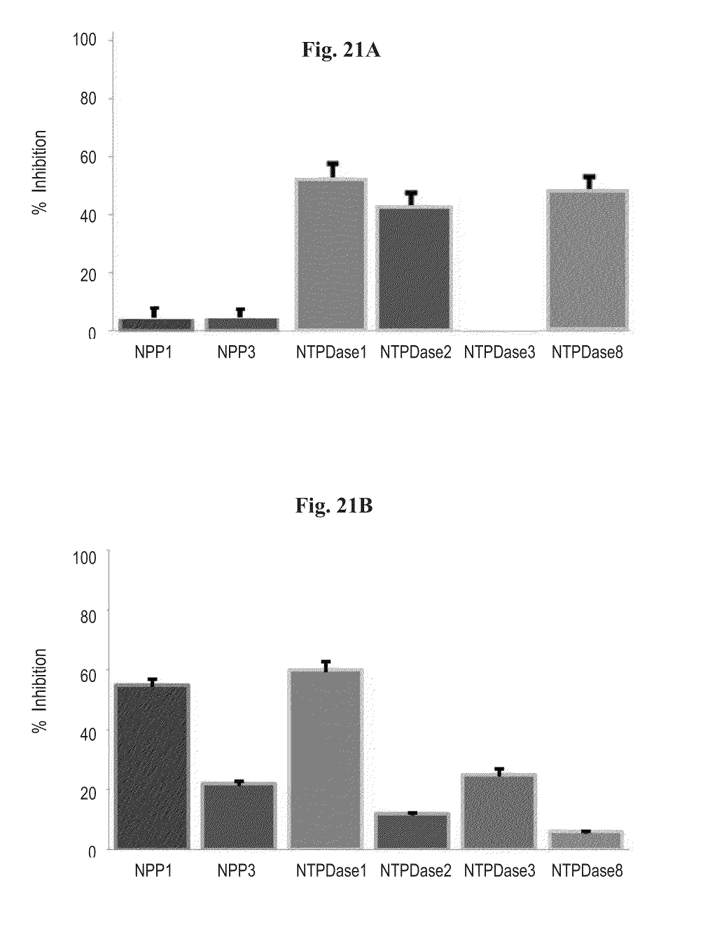

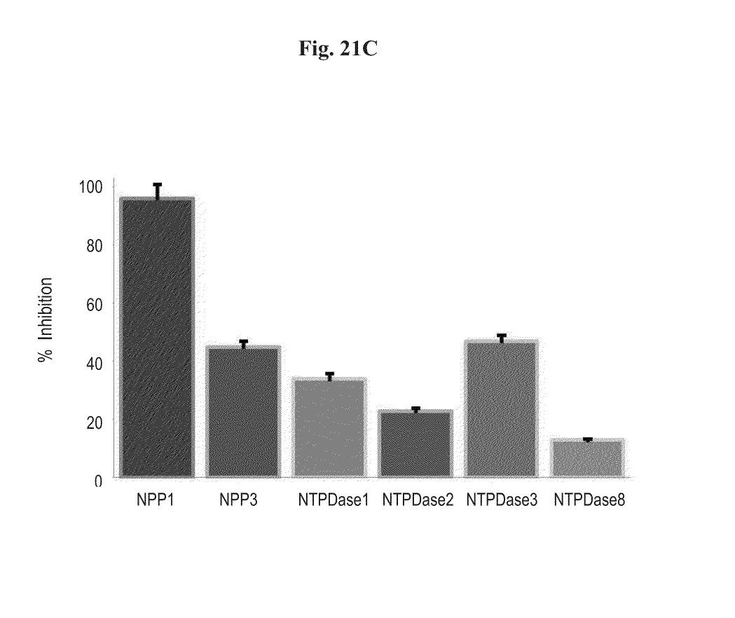

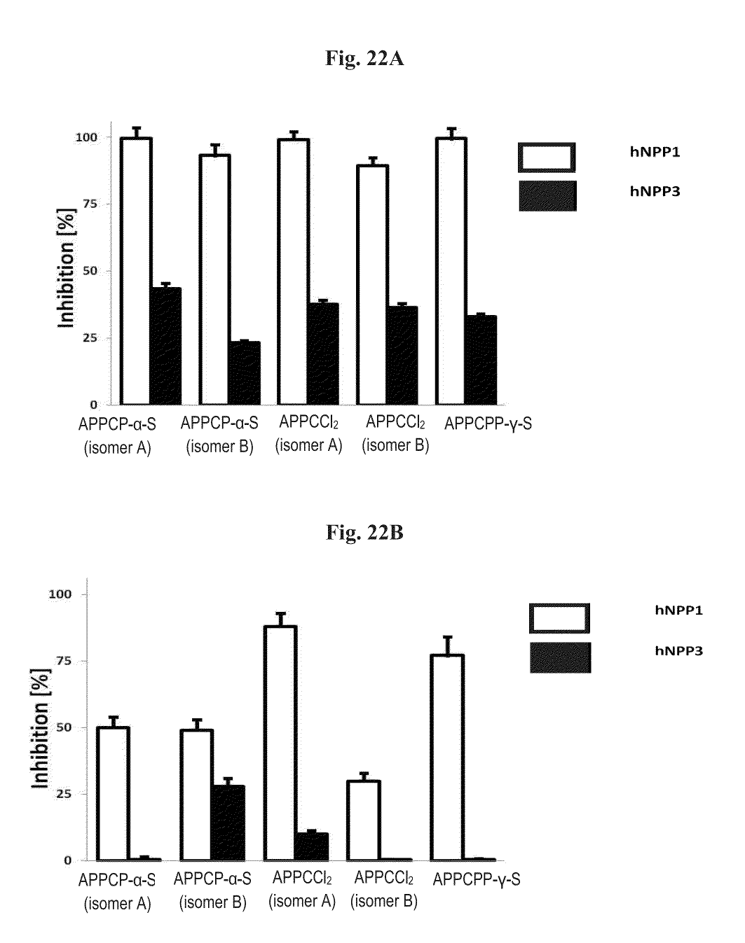

As shown in FIGS. 21A-21C, at 100 μM, adenosine-5′-tetrathiobisphosphonate inhibited NPP1 and NPP3 by 4% and 7%, respectively. In contrast, NTPDase1, 2 and 8 were inhibited by 54%, 42%, and 49%, respectively. Adenosine-5′-tetrathiobis phosphonate thus does not inhibit NPP1, and it is also not an NTPDase1 selective inhibitor. Di-adenosine-5′,5″-tetrathiobisphosphonate inhibited the pnp-TMP hydrolysis by NPP1 and NPP3 at ˜60% and 20%, respectively. Likewise, this analogue inhibited the hydrolysis of ATP by NTPDase1 by ˜60%; however, NTPDase2, 3 and 8 were inhibited by 5-20% only, indicating that di-adenosine-5′,5″-tetrathiobisphosphonate is neither a potent nor selective NPP1 inhibitor, as it inhibits both NPP1 and NTPDase1 by ca. ˜60%. It is thus concluded that although di-adenosine-5′,5″-tetrathiobisphosphonate was found to be a highly chemically and metabolically stable analogue, possibly due the methylene group replacing the phosphate bridging oxygen, it cannot be applied as a NPP1 inhibitor due to lack of protein selectivity. At 100 μM, ADP-β-S inhibited NPP1 by 95%, while NPP3 and NTPDase1, 2, 3 and 8 were inhibited by less than 50%. However, ADP-β-S is also a very good agonist of P2Y_{1,12,13} receptors. This protein-inselectivity precludes the use of ADP-β-S as a NPP1 inhibitor.

### Example 22

### APPCP-α-S, APPCCl₂P-α-S and APCPP-γ-S are not Substrates of NTPDase-1,2,3,8, NPP1,3, or TNAP

Experiments were conducted with protein extracts from COS-7 cells transfected separately with an expression vector encoding each ectonucleotidase i.e., NPP1, NPP3 and NTPDase1, -2, -3, and -8. The protein extracts of non-transfected COS-7 cells exhibited less than 5% NTPDase or NPP activity compared with COS-7 cells transfected with NTPDases or NPPs, thus allowing the analysis of each ectonucleotidase in its native membrane bound form.

APPCP-α-S, APPCCl₂P-α-S and APCPP-γ-S (100 μM, n=3) were stable to hydrolysis by NTPDase1, -2, -3 and -8 when compared to ATP (4.4-5.5% hydrolysis over 1 h, Table 4). APPCCl₂P-α-S and APCPP-γ-S (100 μM) also were neither catabolized by NPP1 nor by NPP3. APPCP-α-S isomer A (100 μM) was fully stable to NPP1 hydrolysis and hardly hydrolysed by NPP3 over 1 h (˜1%) compared to the physiological substrate ATP, and APPCP-α-S isomer B was weakly hydrolyzed by both NPP1 and NPP3 (˜4%).

The metabolic stability of APPCCl₂P-α-S (isomer A) and APCPP-γ-S was further proven by their resistance to enzymatic hydrolysis by TNAP. APPCCl₂P-α-S (isomer A) was fully stable to TNAP hydrolysis during 1 h vs. 100% hydrolysis of ATP, and APCPP-γ-S was negligibly hydrolysed (2.5%) (data not shown).

TABLE 4
Hydrolysis of APPCP-α-S, APPCCl₂P-α-S and APCPP-γ-S by human
ectonucleotidases

| Human | APPCP-α-S | | APPCCl₂P-α-S | | |
|---|---|---|---|---|---|
| ectonucleotidase | A | B | A | B | APCPP-γ-S |
| NTPDase1 | 4.4 ± | 5.4 ± 0.2 | 5.3 ± 0.2 | 5.4 ± | 5.3 ± 0.2 |
| | 0.2 | | | 0.2 | |
| NTPDase2 | 4.7 ± | 5.5 ± 0.3 | 5.2 ± 0.1 | 5.5 ± | 4.7 ± 0.2 |
| | 1.7 | | | 0.2 | |
| NTPDase3 | 4.2 ± | 4.8 ± 0.2 | 5.2 ± 0.2 | 5.4 ± | 4.8 ± 0.2 |
| | 0.2 | | | 0.2 | |
| NTPDase8 | 4.4 ± | 5.3 ± 0.2 | 4.3 ± 0.1 | 5.3 ± | 5.2 ± 0.2 |
| | 0.2 | | | 0.2 | |

The adenosine triphosphate analogues were incubated in the presence of the indicated ectonucleotidases at the concentration of 100 γM. The activity with 100 μM ATP was set as 100% which was 807±35, 1051±45, 240±17 and 122±7 [nmol Pi·min⁻¹·mg protein⁻¹] for NTPDase1, -2, -3 and -8, respectively. For NPP1 and NPP3, 100% of the activity with 100 γM ATP as the substrate was 67±5 and 54±2 [nmol of nucleotide·min⁻¹·mg protein⁻¹], respectively. Data presented are the means±SD of results from 3 experiments carried out in triplicates.

### Example 23

### APPCP-α-S, APPCCl₂P-α-S and APCPP-γ-S are not Selective Inhibitors of NPP1

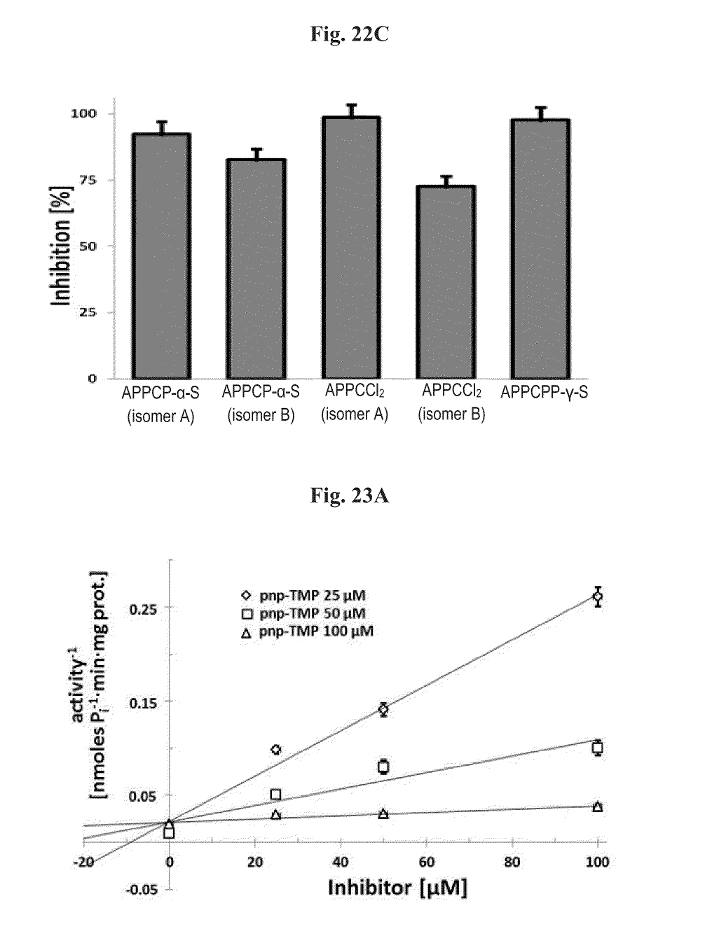

The effect of APPCP-α-S, APPCCl₂P-α-S and APCPP-γ-S on NPP and NTPDase activities and their selectivity were tested using a synthetic substrate (pNP-TMP) as well as a natural substrate (ATP), respectively. APPCP-α-S, APPCCl₂P-α-S and APCPP-γ-S at (100 μM, n=3) effectively inhibited pNP-TMP (100 μM) hydrolysis by NPP1 by over 90% (FIG. 22A). The hydrolysis of the physiological substrate ATP by NPP1 was inhibited more potently by APPCCl₂P-α-S (isomer A) and APCPP-γ-S vs. APPCP-α-S (isomers A and B) and APPCCl₂P-α-S (isomer B) (FIG. 22B). Similar inhibition was observed when osteocarcinoma cells (HTB85 cells, also known as SaOS 2) were used as a native source of NPP1 (FIG. 22C). APPCP-α-S, APPCCl₂P-α-S and APCPP-γ-S, at 100 μM, were NPP1 selective inhibitors, since they inhibited NPP3 activity by only 23-43% (FIG. 22A-B).

APPCP-α-S (isomers A and B), APPCCl₂P-α-S (isomers A and B) did not interfere with the hydrolysis of ATP by human NTPDase1, -2, -3 and -8 (Table 2). Analogue APCPP-γ-S at 100 μM inhibited human NTPDase1 and -3 by 60% and 40%, respectively (Table 5). Both compounds APPCCl₂P-α-S (isomere A) and APCPP-γ-S at 100 μM showed low inhibition of TNAP, 17% and 8% respectively.

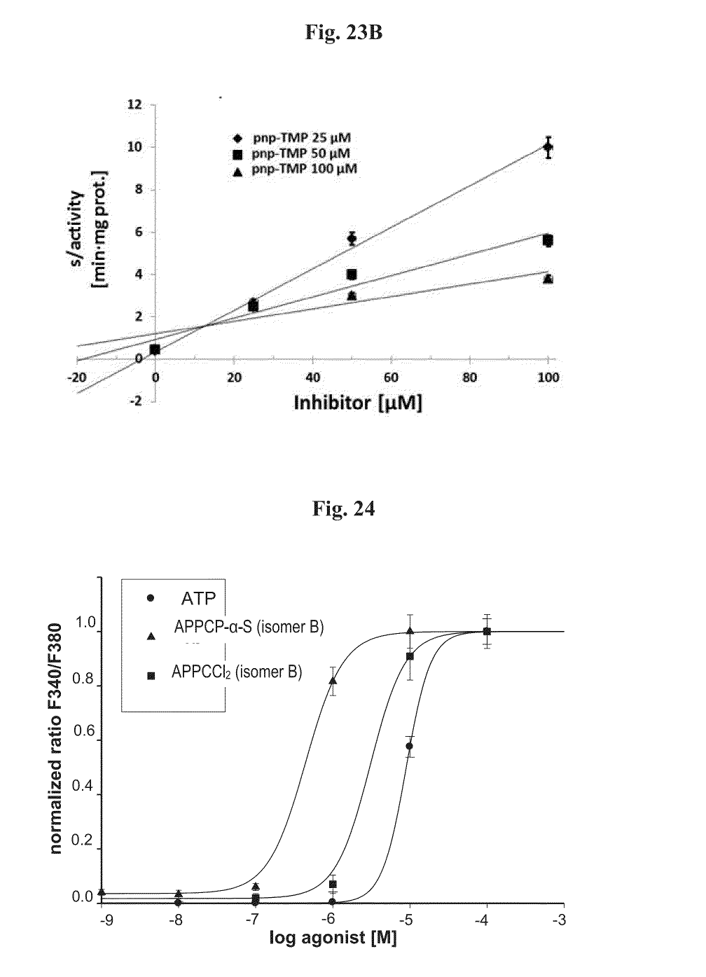

We have estimated IC₅₀ (a parameter that shows the ability of a molecule to inhibit an enzyme under specific conditions and substrate concentration), and inhibition constant K (a kinetic parameter that represents an absolute value for each tested inhibitor) towards NPP1. The kinetic parameters indicate that analogue APCPP-γ-S is the most potent inhibitor of NPP1 with Kᵢ value of 20 nM (Table 6, FIG. 23). Analogue APPCCl₂P-α-S (isomer A) was also a good NPP1 inhibitor exhibiting a Kᵢ value of 685 nM. Under these experimental conditions the IC₅₀s of both analogues were also the lowest being 390 nM and 600 nM for APCPP-γ-S and APPCCl₂P-α-S (isomer A), respectively (Table 6). Using the methods of Dixon (FIG. 23A and Table 6) and Cornish-Bowden (FIG. 23B and Table 6) that estimate dissociation constant for EIS complex (Kᵢ′), we determined that the inhibitors APPCP-α-S, APPCCl₂P-α-S and APCPP-γ-S presented in Table 6, showed mixed type inhibition, predominantly competitive.

TABLE 5
The effect of APPCP-α-S, APPCCl₂P-α-S and APCPP-γ-S on human
NTPDase1, -2, -3, -8 activity

| | APPCP-α-S | | APPCCl₂P-α-S | | |
|---|---|---|---|---|---|
| Enzyme | A | B | A | B | APCPP-γ-S |
| NTPDase1 | 58.4 ± 2.1 | 0.5 ± 0.02 | 0.5 ± 0.01 | 21.6 ± 1.0 | 18.7 ± 0.8 |
| NTPDase2 | 16.3 ± 0.6 | 9.9 ± 0.4 | 11.2 ± 0.5 | 12.7 ± 0.5 | 15.4 ± 0.7 |
| NTPDase3 | 40.2 ± 2.0 | 18.7 ± 0.8 | 21.8 ± 1.0 | 24.0 ± 1.2 | 26.8 ± 1.1 |
| NTPDase8 | 7.0 ± 0.3 | 0.5 ± 0.01 | 4.9 ± 0.2 | 1.5 ± 0.06 | 0.50 ± 0.02 |

ATP was used as the substrate in the presence of one of the analogues tested. Both substrate and the analogues were studied at 100 μM. The 100% activity was set with the substrate ATP alone which was 807±35, 1051±45, 240±17 and 122±7 [nmol Pi·min⁻¹·mg protein⁻¹] for NTPDase1, -2, -3 and -8, respectively. Data presented are the mean±SD of 3 experiments carried out in triplicates.

TABLE 6
Kinetic parameters and IC50 of NPP1 inhibition

| | APPCP-α-S | | APPCCl₂P-α-S | | |
|---|---|---|---|---|---|
| Inhibitor | A | B | A | B | APCPP-γ-S |
| Kᵢ [μM] | 4.5 ± 0.03 | 1.3 ± 0.01 | 0.685 ± 0.005 | 15.2 ± 0.1 | 0.02 ± 0.0001 |
| Kᵢ′ [μM] | 4.5 ± 0.003 | −71.5 ± 0.5 | −12.5 ± 0.1 | −192.0 ± 1 | <9.0 ± 0.05 |
| IC₅₀ [μM] | 16.3 ± 0.04 | 18.7 ± 0.03 | 0.6 ± 0.01 | 31.2 ± 0.1 | 0.39 ± 0.001 |

For Kᵢ and Kᵢ′ determinations, pNP-TMP (substrate) and the analogues tested were used in the concentration range of 2.5·10⁻⁵-1·10⁻³ M. For IC₅₀ determinations, pNP-TMP concentration was 5·10⁻⁵ M and inhibitors ranged from 5·10⁻⁷ to 1·10⁻³ M. All experiments were performed three times in triplicates.

### Example 24

### The Efficacy of APCPP-γ-S in a Mouse Model for AD

Six-month old homozygous 3×Tg-AD mice (known as a mouse model of Alzheimer's disease) are daily injected (I.P.) during three months with either APCPP-γ-S or its prodrug 1-D-glucosyl-Pγ-APCPP-γ-S (2 mg/kg and 20 mg/kg), wherein mice treated with Menamtine (30 mg/kg) are used as positive controls. Age-matched mice injected with PBS, as well as age-matched non-Tg mice, are used as controls.

Animal Behavioral Testing:

Morris Water Maze. The Morris Water Maze (MWM) is used, and the parameters measured during the probe trial include (1) initial latency to cross the platform location; (2) number of platform location crosses; and (3) time spent in the quadrant opposite to the target quadrant. Novel object recognition. This task measures the time spent exploring the familiar object and the novel object is calculated. Time spent with the novel object as compared to time spent with both objects is used as memory index.

Protein Analysis, Immunohistochemistry.

Mice are sacrificed and their brains are tested with the following antibodies: Anti-Aβ (6E10), anti-APP (22C11), and Aβ (_{40/42}) anti-Tau HT7, anti-GSK3β-p, anti-β-actin, anti-p38, and anti-CDK5.

PKC and GSK3β Activities.

PKC activity is measured using a kit from Streegen (Victoria, Canada) and the GSK3β activity is measured using a kit from SIGMA.

### Example 25

### APPCCl₂P-α-S, an NPP1 Inhibitor, is Effective in Reducing ATP Hydrolysis and PPi and CPPD Formation in Human MVs, Chondrocytes and Cartilage

### Hydrolysis of Extracellular ATP in the Presence of Human Chondrocyte Culture

In this study, the metabolic hydrolysis of ATP was evaluated. ATP and APPCCl₂P-α-S (isomer A) were taken at a final concentration of 100 μM and incubated in the absence or the presence of human chondrocyte cells for 0, 3, 6 and 8 h. Following incubation, the samples were collected and heated at 80° C. for 15 min and extracted with chloroform, applied onto an anion exchange cartridge, and freeze-dried. The resulting residue was analyzed by HPLC. The hydrolysis rate of ATP was determined by measuring the change in the integration of the respective HPLC peaks with time.

As shown in FIG. 25, during 8 h of incubation in the absence of chondrocyte cells, the amount of ATP decreased by 18%, while in the presence of chondrocyte cells it decreased by 56%. Co-application of the NPP1 inhibitor APPCCl₂P-α-S (isomer A) and ATP to human chondrocyte cells resulted in only 22% degradation, indicating that APPCCl₂P-α-S (isomer A) inhibits several enzymes involved in nucleotide degradation.

### Evaluation of Inhibition of ATP Hydrolysis in Matrix Vesicles by APPCCl₂P-α-S

Increased chondrocyte PPi production, and PPi-generating NPP1 activity are linked with CPPD crystal deposition disease, common in aging and osteoarthritic cartilage. In this study, NTMP (p-nitrophenyl-tymidine-5′-monophosphate) was used as a substrate that mimics endogenous ATP. Its hydrolysis by endogenous enzyme (NPP1) from matrix vesicles (MVs), human chondrocytes or cartilage pieces results in elevated OD at 405 nm.

In order to test the ability of the NPP1 inhibitor APPCCl₂P-α-S (isomer A) to inhibit NTMP hydrolysis in MVs, MVs at 2.6 mg/ml (final concentration) were incubated with 0.1 mM NTMP in 2-[(2-Hydroxy-1,1-bis(hydroxymethyl)ethyl)amino]ethanesulfonic acid (TES; Sigma catalog number T1375), pH 7.37, in the presence or absence of 0.1 mM of said NPP1 inhibitor for 0, 15′, 30′, 1, 2, 4, 8 hours at 37° C. At each time point the reaction was read at 405 nm.

In order to test the ability of the NPP1 inhibitor to inhibit NTMP hydrolysis in human chondrocyte cells, human chondrocytes 1.5×10⁴ per well were seeded in 96 well plate in 100 μl DMEM/10% fetal calf serum (FCS). One day before the experiment, the medium was changed to DMEM only (w/o phenol red). Human chondrocytes were incubated with 0.1 mM NTMP in TES, pH 7.37, in the presence or absence of 0.1 mM of the NPP1 inhibitor for 0, 15′, 30′, 1, 2, 4, 6, 18 hours at 37° C. At each time point the reaction was read at 405 nm.

In order to test the ability of the NPP1 inhibitor to inhibit NTMP hydrolysis in cartilage pieces, 12 pieces of cartilage of a uniform diameter were weighed and placed in wells of a 96-well plate overnight in DMEM w/o phenol red with penicillin-streptomycin-glutamine (PSG). The pieces of cartilage were incubated with 0.1 mM NTMP in TES, pH 7.37, in the presence or absence of 0.1 mM of the NPP1 inhibitor for 0, 15′, 30′, 1, 2, 4, 6, 8 hours at 37° C. At each time point 200 μl were removed from each well and the reaction was read at 405 nm. In all experiments, the NPP1 inhibitor inhibited the hydrolysis of NTMP as compared to non-treated matrices.

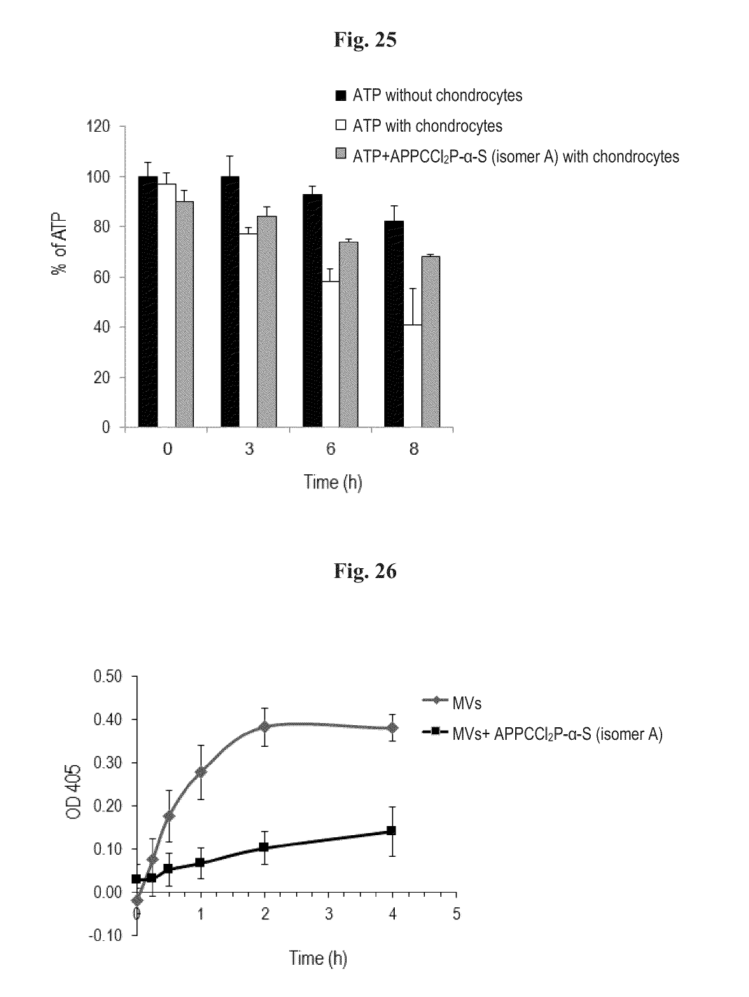

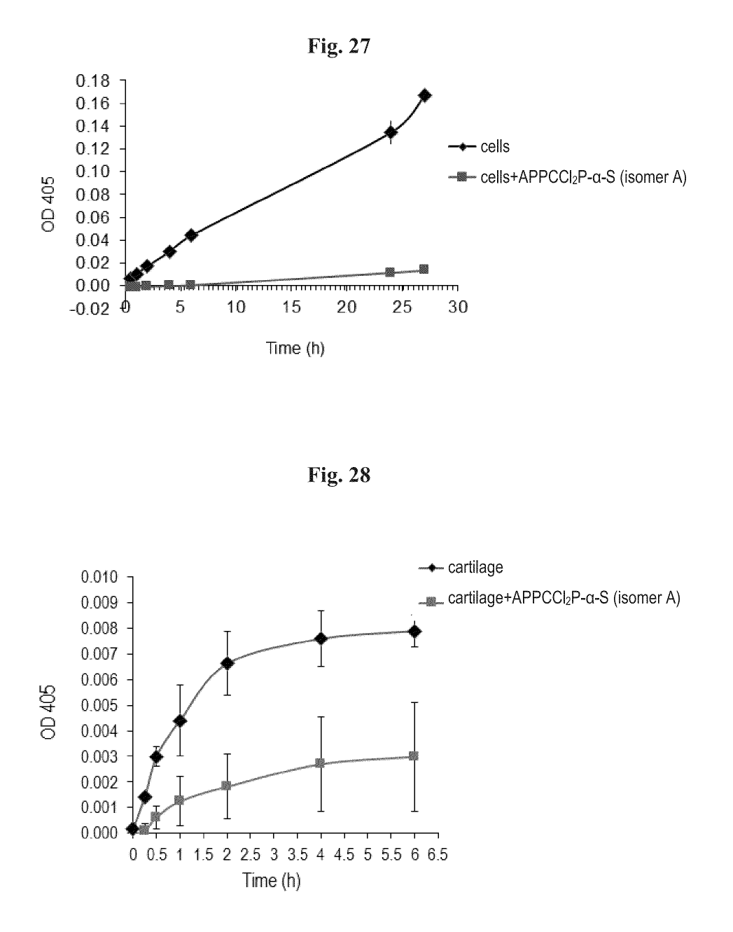

FIGS. 23-25 show the ability of APPCCl₂P-α-S (isomer A) to inhibit the hydrolysis of NTMP in MVs (FIG. 26), human chondrocyte cells (FIG. 27) and cartilage pieces (FIG. 28).

### Determination of the Amount of Pyrophosphate Obtained Due to ATP Hydrolysis in the Presence of Chondrocytes

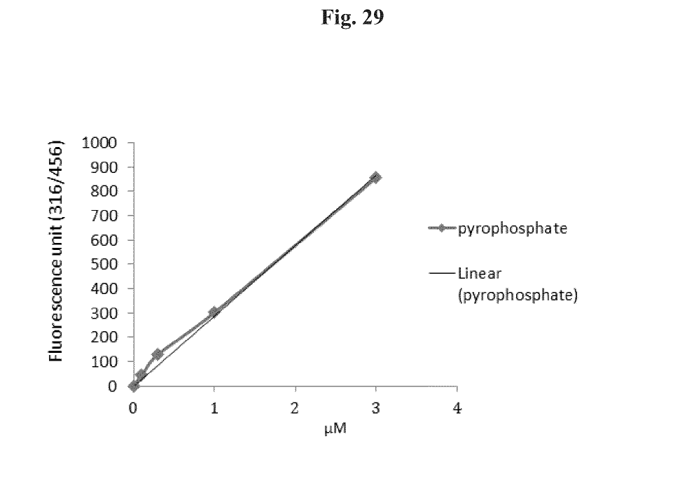

The assay used in this study is a direct measurement of pyrophosphate (PPi, produced by ATP hydrolysis) by a fluorogenic pyrophosphate sensor that has its fluorescence intensity proportionally dependent upon the concentration of pyrophosphate. FIG. 29 shows a standard curve of pyrophosphate as measured by pyrophosphate assay kit, which was used to evaluate PPi concentration in samples. ATP was incubated in the presence or absence of chondrocyte cells to evaluate the amount of pyrophosphate formed due to ATP degradation, and in a different experiment, ATP was incubated together with APPCCl₂P-α-S (isomer A) in the presence of chondrocytes. As found, 0.26 μM pyrophosphate was formed after incubation of ATP without chondrocytes during 8 h, while 0.46 μM pyrophosphate was formed after incubation of ATP with chondrocytes. Co-application of ATP and APPCCl₂P-α-S (isomer A) in the presence of chondrocytes for 8 h resulted in only 0.029 μM PPi.

### Evaluation of CPPD Formation in MVs in the Presence of ATP and APPCCl₂P-α-S by FTIR Analysis

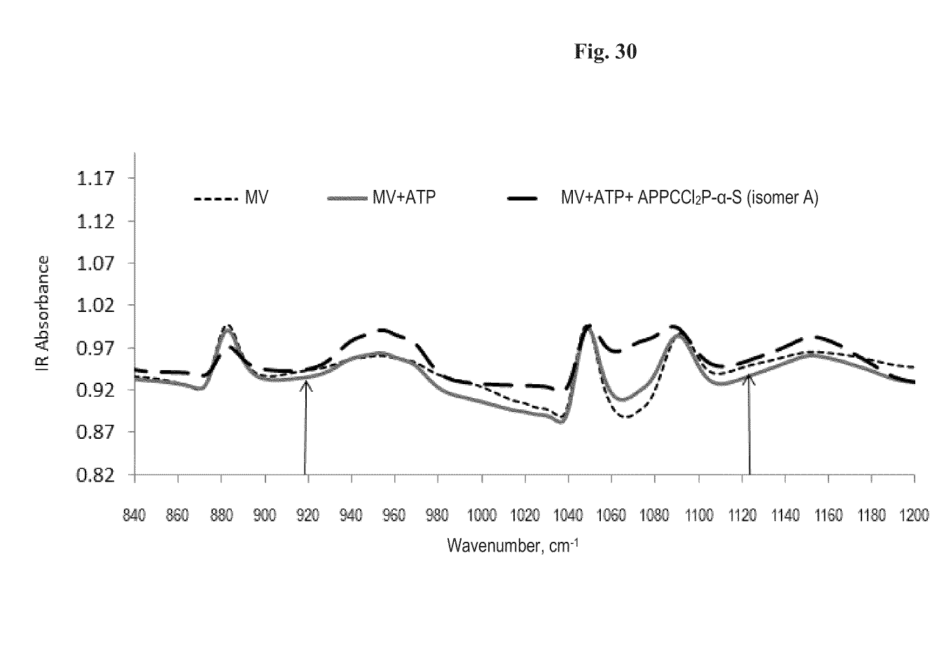

In this study, FTIR has been used to characterize the mineral phase associated with MVs in the presence of ATP and APPCCl₂P-α-S (isomer A). Comparison of FTIR spectra of MV and MV+ATP indicated that the mineral phase associated with MVs displays characteristic bands near 920 and 1125 cm⁻¹ (FIG. 30) indicating the presence of CPPD. The band intensity of MV+ATP+APPCCl₂P-α-S (isomer A) is similar to that of MVs alone, indicating a decrease in production of CPPD.

### APPENDIX A

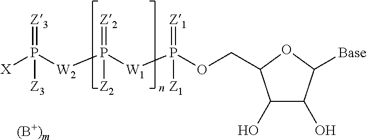

| Compound | Base | Z′₁, Z′₂, Z′₃ | Z1, Z₂, Z₃ | W₁ | W₂ | X | n |
|---|---|---|---|---|---|---|---|
| ATP | adenine | O, O, O | O⁻, O⁻, O⁻ | O | O | O⁻ | 1 |
| GTP | guanine | O, O, O | O⁻, O⁻, O⁻ | O | O | O⁻ | 1 |
| ATP-γ-S | adenine | O, O, S | O⁻, O⁻, O⁻ | O | O | O⁻ | 1 |
| GTP-γ-S | guanine | O, O, S | O⁻, O⁻, O⁻ | O | O | O⁻ | 1 |
| ADP | adenine | O, —, O | O⁻, —, O⁻ | — | O | O⁻ | 0 |
| GDP | guanine | O, —, O | O⁻, —, O⁻ | — | O | O⁻ | 0 |
| ADP-β-S | adenne | O, —, S | O⁻, —, O⁻ | — | O | O⁻ | 0 |
| GDP-β-S | guanine | O , —, S | O⁻, —, O⁻ | — | O | O⁻ | 0 |
| APCPP-γ-S | adenine | O, O, S | O⁻, O⁻, O⁻ | CH₂ | O | O⁻ | 1 |
| APPCP-α-S | adenine | S, O, O | O⁻, O⁻, O⁻ | O | CH₂ | O⁻ | 1 |
| APPCCl₂P-α-S | adenine | S, O, O | O⁻, O⁻, O⁻ | O | CCl₂ | O⁻ | 1 |
| AP₂A | adenine | O, —, O | O⁻, —, O⁻ | — | O | adenine | 0 |
| 1-D-glucosyl-Pβ-ADP-β-S | adenine | O, —, S | O⁻, —, O⁻ | O | O | glucose | 0 |
| 1-D-glucosyl-Pγ-APCPP-γ-S | adenine | O, O, S | O⁻, O⁻, O⁻ | CH₂ | O | glucose | 1 |
| APCP-α,α′,β,β′-tetra-S | adenine | S, —, S | S⁻, —, S⁻ | — | CH₂ | O⁻ | 0 |
| UPCP-α,α′,β,β′-tetra-S | uracil | S, —, S | S⁻, —, S⁻ | — | CH₂ | O⁻ | 0 |
| APCPA-α,α′,β,β′-tetra-S | adenine | S, —, S | S⁻, —, S⁻ | — | CH₂ | adenine | 0 |
| UPCPU-α,α′,β,β′-tetra-S | uracil | S, —, S | S⁻, —, S⁻ | — | CH₂ | uracil | 0 |

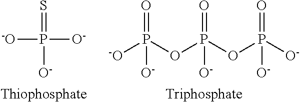

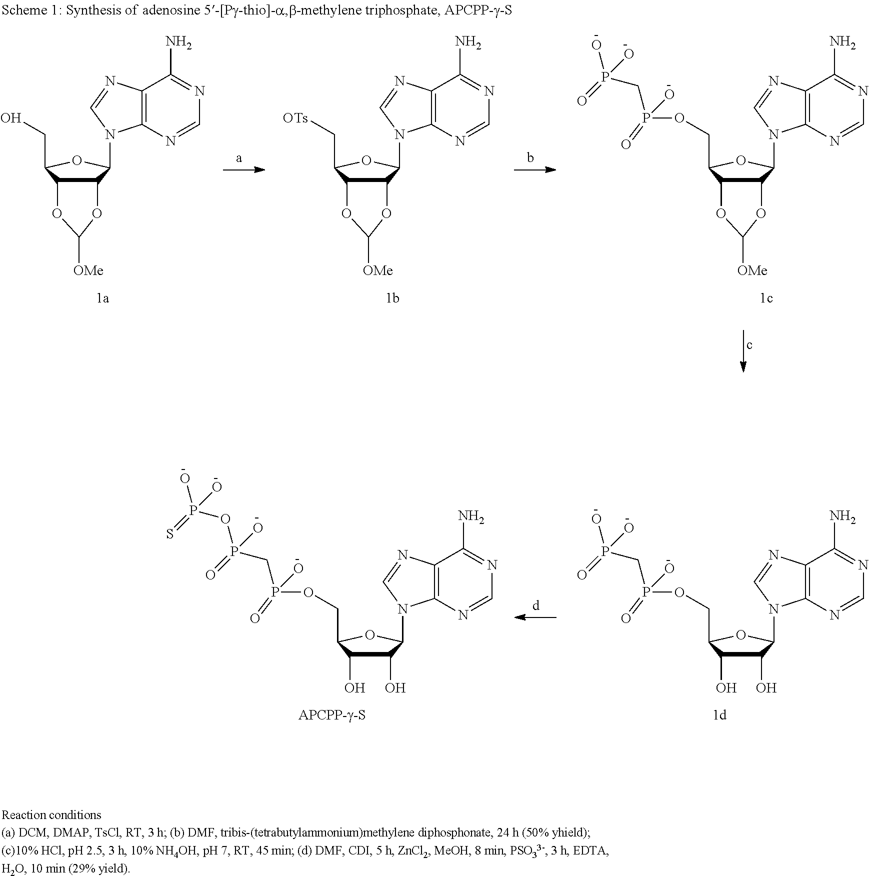

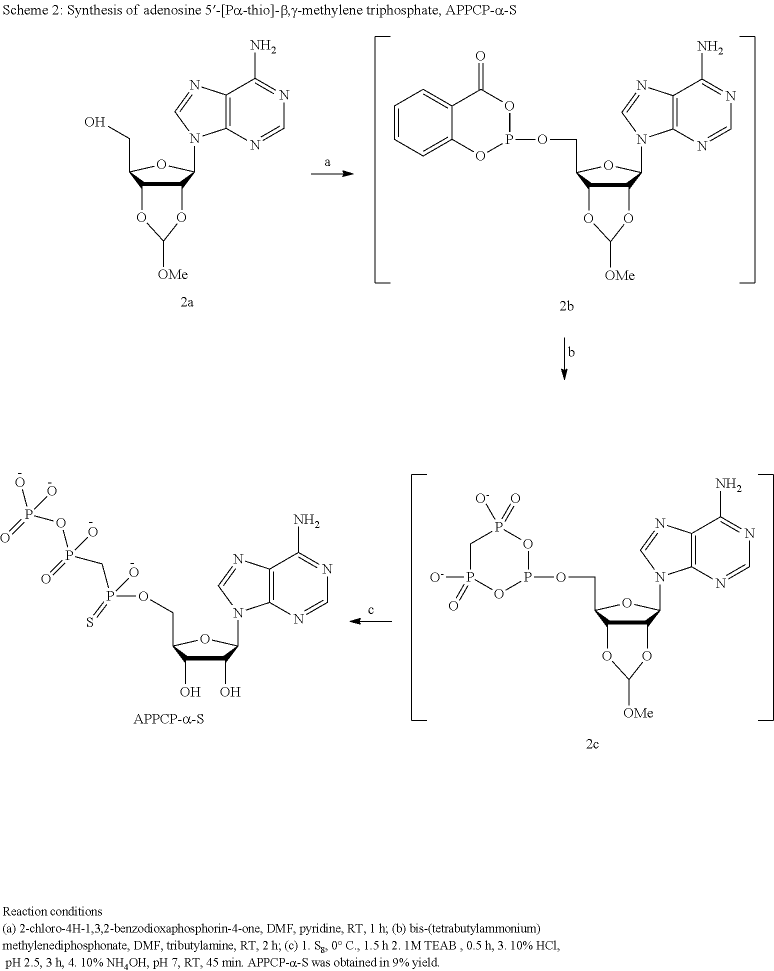

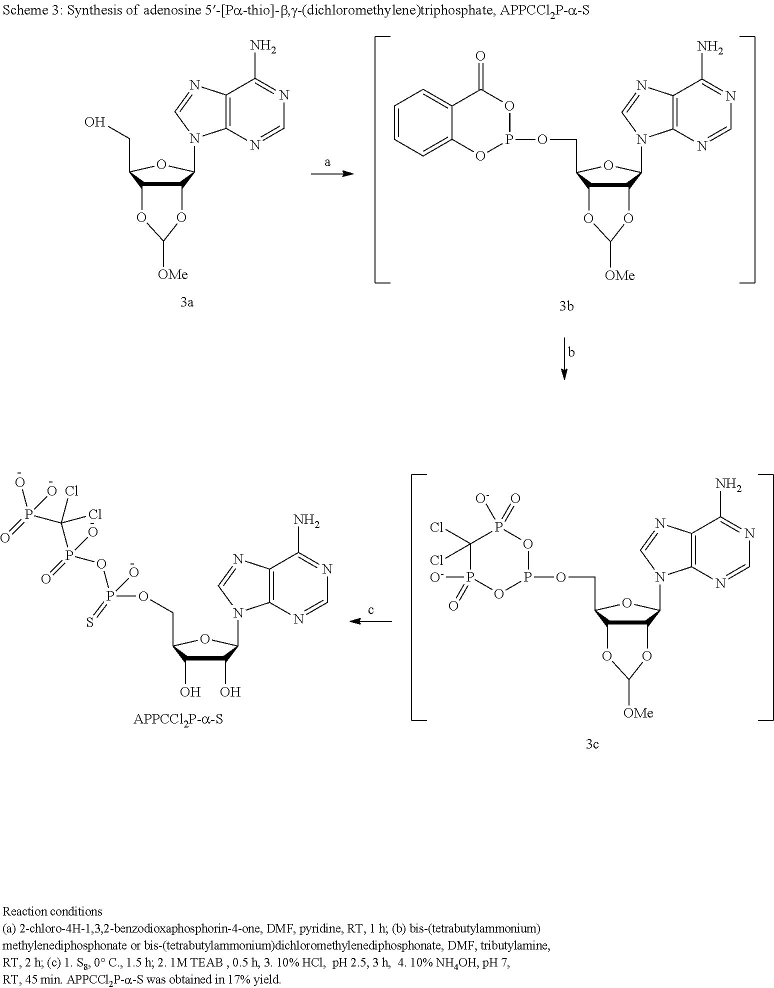

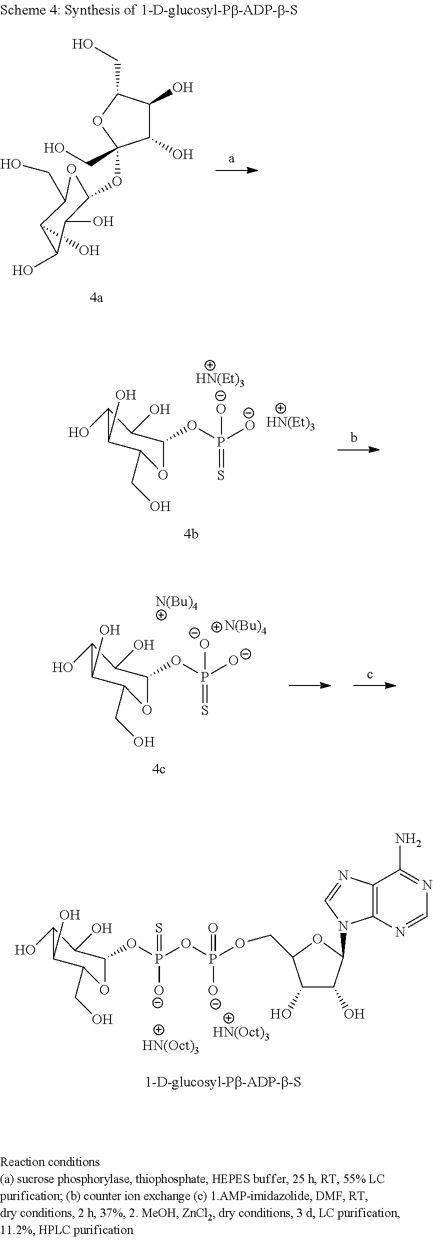

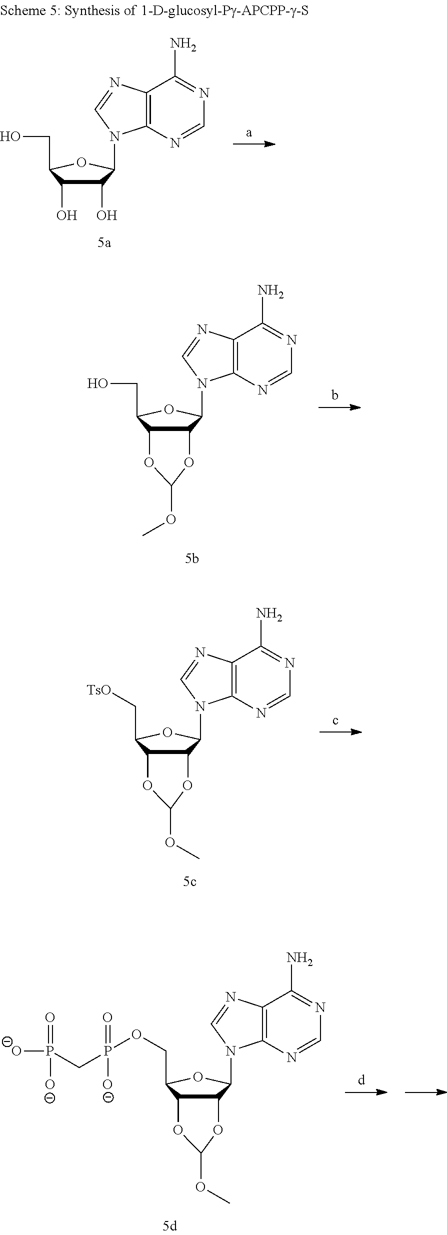

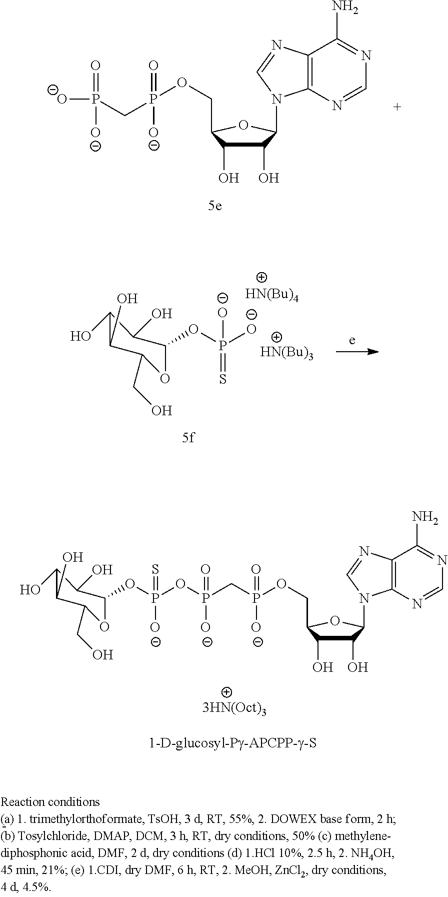

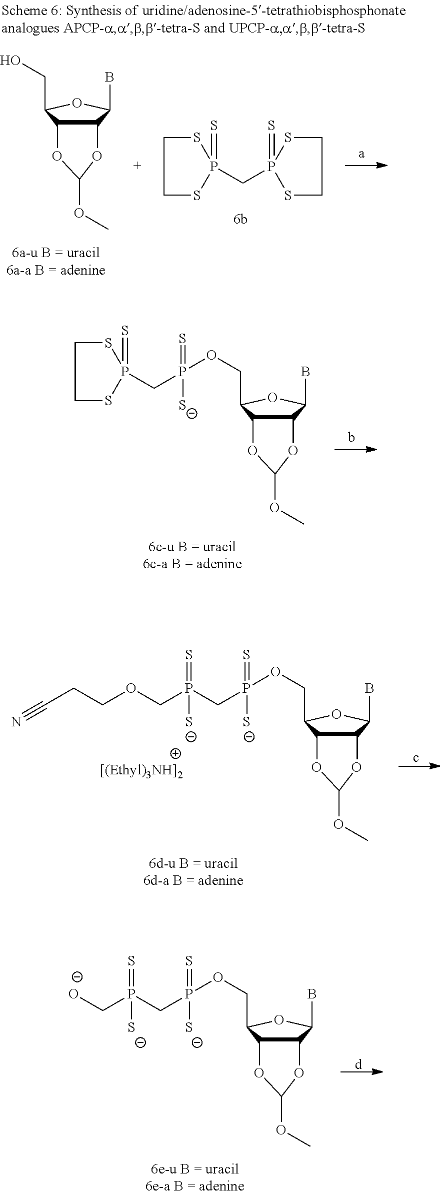

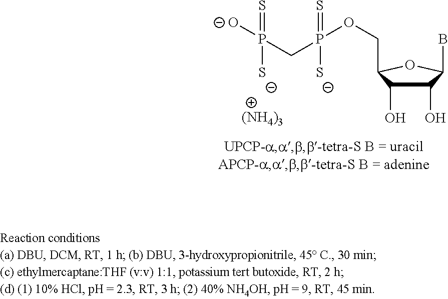

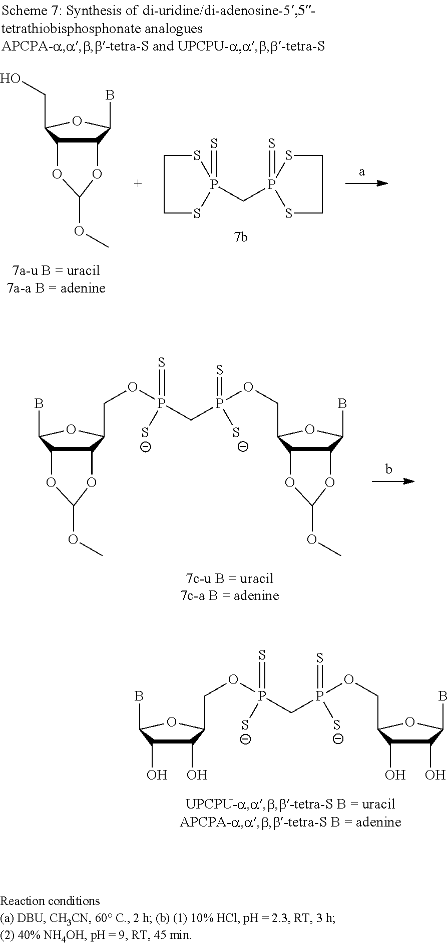

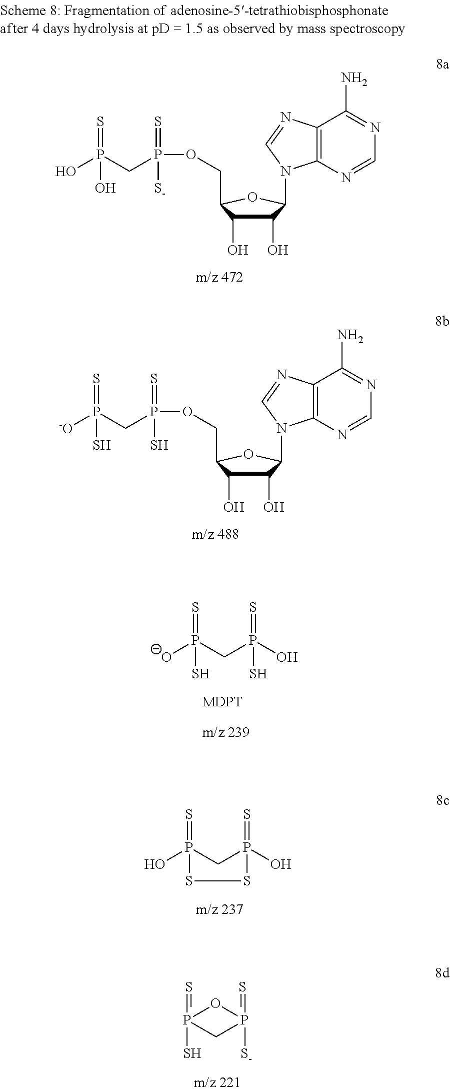

### REFERENCES

Abramov, E., Dolev, I., Fogel, H., Ciccotosto, G. D., Ruff, E., Slutsky, I., Nat. Neurosci., 2009, 12, 1567-1576
Adlar, P. A., Cherny, R. A., Finkelstein, D. I., Gautier, E., Robb, E., Cortes, M., Volitakis, I., Liu, X., Smith, J. P., Perez, K., Laughton, K., Li, Q. X., Charman, S. A., Nicolazzo, J. A., Wilkins, S., Deleva, K., Lynch, T., Kok, G., Ritchie, C. W., Tanzi, R. E., Cappai, R., Masters, C. L., Barnham, K. J., Bush, A. I., Neuron, 2008, 59, 43-55
Akiyama, H., Barger, S., Barnum, S., Bradt, B., Bauer, J., Cole, G. M., Cooper, N. R., Eikelenboom, P., Emmerling, M., Fiebich, B. L., Finch, C. E., Frautschy, S., Griffin, W. S. T., Hampel, H., Hull, M., Landreth, G., Lue, L. F., Mrak, R., Mackenzie, I. R., McGeer, P. L., O'Banion, M. K., Pachter, J., Pasinetti, G., Plata-Salaman, C., Rogers, J., Rydel, R., Shen, Y., Streit, W., Strohmeyer, R., Tooyoma, I., Van Muiswinkel, F. L., Veerhuis, R., Walker, D., Webster, S., Wegrzyniak, B., Wenk, G., Wyss-Coray, T., Neurobiol. Aging, 2000, 21, 383-421
Amir, A., Sayer, A. H., Zagalsky, R., Shimon, L. J., Fischer, B., J Org. Chem., 2013, 78(2), 270-277
Atwood, C. S., Moir, R. D., Huang, X., Scarpa, R. C., Bacarra, N. M. E., Romano, D. M., Hartshorn, M. A., Tanzi, R. E., Bush, A. I., J. Biol. Chem., 1998, 273, 12817-12826
Atwood, C. S., Perry, G., Zeng, H., Kato, Y., Jones, W. D., Ling, K. Q., Huang, X., Moir, R. D., Wang, D., Sayre, L. M., Smith, M. A., Chen, S. G., Bush, A. I., Biochemistry, 2004, 43, 560-568
Barnea, A., Cho, G., Katz, B. M., Brain Res., 1991, 541, 93-97
Barr, A. J., Conaghan, P. G., Medicographia, 2013, 35, 189-196
Bartolini, M., Bertucci, C., Bolognesi, M. L., Cavalli, A., Melchiorre, C., Andrisano, V., ChemBioChem, 2007, 8, 2152-2161
Baruch-Suchodolsky, R., Fischer, B., J. Inorg. Biochem., 2008, 102, 862-881
Baruch-Suchodolsky, R., Fischer, B., Biochemistry, 2009, 48, 4354-4370
Baykov, A. A., Evtushenko, O. A., Avaeva, S. M., Analytical Biochemistry, 1988, 171, 266-270
Belli, S. I., Goding, J. W., European Journal of Biochemistry, 1994, 226, 433-443
Bombois, S., Maurage, C. A., Gompel, M., Deramecourt, V., Mackowiak-Cordoliani, M. A., Black, R. S., Lavielle, R., Delacourte, A., Pasquier, F., Arch. Neurol., 2007, 64, 583-587
Brenner, A. J., Harris, E. D., Anal. Biochem., 1995, 226, 80-84
Bush, A. I., Trends Neurosci., 2003, 26, 207-214
Cherny, R. A., Adlard, P. A., Finkelstein, D. I., Gautier, E., Robb, E., Cortes, M., Volitakis, I., Liu, X., Smith, J. P., Perez, K., Laughton, K., Li, Q. X., Charman, S. A., Nicolazzo, J. A., Wilkins, S., Deleva, K., Lynch, T., Kok, G., Ritchie, C. W., Tanzi, R. E., Cappai, R., Masters, C. L., Barnham, K. J., Bush, A. I., Neuron, 2008, 59, 43-55
Curtain, C. C., Ali, F., Volitakisi, I., Chernyi, R. A., Norton, R. S., Beyreuther, K., Barrow, C. J., Mastersi, C. L., Bushi, A. I., Barnham, K. J., J. Biol. Chem., 2001, 276, 20466-20473
Dai, X., Sun, Y., Gao, Z., Jiang, Z., J. Mol. Neurosci., 2009, 41, 66-73 Doraiswamy, P. M., Finefrock, A. E., Lancet Neurol., 2004, 3, 431-434
Ecke, D., Tulapurkar, E. M., Nahum, V., Fischer, B., Reiser, G., British Journal of Pharmacology, 2006, 149(4), 416-423
Ecke, D., Hanck, T., Tulapurkar, E. M., Schafer, R., Kassack, M., Stricker, R, Reiser, G., Biochemical Journal, 2008, 409(1), 107-116
Faller, P., Hureau, C., Dalton Trans., 2009, 1080-1094
Faller, P., ChemBioChem, 2009, 10, 2837-2845
Fezoui, Y., Hartley, D. M., Harper, J. D., Khurana, R., Walsh, D. M., Condron, M. M., Selkoe, D. J., Lansbury, P. T., Fink, A. L., Teplow, D. B., Amyloid, 2000, 7, 166-178
Francis, P. T., Palmer, A. M., Snape, M., Wilcook, G. K., J. Neurol., Neurosurg. Psychiatry, 1999, 66, 137-147
Fujita, T., Tozaki-Saitoh, H., Inoue, K., Glia, 2009, 57(3), 244-257
Furia, T. E., CRC Handbook of Food Additives, 2^{nd} edn, 1980, vol. 2, pp. 271-294
Garzon-Rodriguez, W., Yatsimirsky, A. K., Glabe, C. G., Bioorg. Med. Chem. Lett., 1999, 9, 2243-2248
Goody, R. S., Eckstein, F., J. Am. Chem. Soc., 1971, 93, 6252-6257
Green, D. E., Bowen, M. L. Scott, L. E., Storr, T., Merkel, M., Bohmerle, K., Thompson, K. H., Patrick, B. O., Schugarb, H. J., Orvig, C., Dalton Trans., 2010, 39, 1604-1615
Huang, X., Atwood, C. S., Moir, R. D., Hartshorn, M. A., Vonsattel, J. P., Tanzi, R. E., Bush, A. I., J. Biol. Chem., 1997, 272, 26464-26470
Iqbal, K., Del, A., Alonso, C., Chen, S., Chohan, M. O., El-Akkad, E., Gong, C. X., Khatoon, S., Li, B., Liu, F., Rahman, A., Tanimukai, H., Grundke-Iqbal, I., Biochim. Biophys. Acta, Mol. Basis. Dis., 2005, 1739, 198-210
Jomova, K., Vondrakova, D., Lawson, M., Valko, M., Mol. Cell. Biochem., 2010, 345, 91-104
Karr, J. W., Akintoye, H., Kaupp, L. J., Szalai, V. A., Biochemistry, 2005, 44, 5478-5487
Kowalska, J., Lewdorowicz, M., Darzynkiewicz, E., Jemielity, J., Tetrahedron Lett., 2007, 48, 5475-5479
Kukulski, F., Levesque, S. A., Lavoie, E. G., Lecka, J., Bigonnesse, F., Knowles, A. F., Robson, S. C., Kirley, T. L., Sevigny, J., Purinergic Signalling, 2005, 1, 193-204
Lakatos, A., Zsigo, E., Hollender, D., Nagy, N. V., Fueloep, L., Simon, D., Bozso, Z., Kiss, T., Dalton Trans., 2010, 39, 1302-1315
Lau, L. F., Brodney, M. A., Curr. Top. Med. Chem., 2008, 2, 1-24
LeVine, H., Dingb, Q., Walkerc, J. A., Vossc, R. S., Augelli-Szafran, C. E., Neurosci. Lett., 2009, 465, 99-103
Lim, G. P., Yang, F., Chu, T., Chen, P., Beech, W., Teter, B., Tran, T., Ubeda, O., Ashe, K. H., Frautschy, S. A., Cole, G. M., J. Neurosci., 2000, 20, 5709-5714
Lomakin, A., Teplow, D. B., Kirschner, D. A., Benedek, G. B., Proc. Natl. Acad. Sci. U.S.A., 1997, 94, 7942-7947
Lovell, M. A., Robertson, J. D., Teesdale, W. J., Campbell, J. L., Markesbery, W. R., J. Neurol. Sci., 1998, 158, 47-52
Miura, T., Suzuki, K., Kohata, N., Takeuchi, H., Biochemistry, 2000, 39, 7024-7031
Ohtsuki, S., Terasaki, T., Pharmaceutical research, 2007, 24(9), 1745-1758
Panza, F., Frisardi, V., Imbimbo, B. P., Capurso, C., Logroscino, G., Sancarlo, D., Seripa, D., Vendemiale, G., Pilotto, A., Solfrizzi, V., CNS Neurosci. Ther., 2010, 16, 272-284
Pratico, D., Trends Pharmacol. Sci., 2008, 29, 609-615
Richter, Y., Fischer, B., JBIC, J. Biol. Inorg. Chem., 2006, 11, 1063-1074
Ritchie, C. W., Bush, A. I., Mackinnon, A., Macfarlane, S., Mastwyk, M., MacGregor, L., Kiers, L., Cherny, R., Li, Q. X., Tammer, A., Carrington, D., Mavros, C., Volitakis, I., Xilinas, M., Ames, D., Davis, S., Beyreuther, K., Tanzi, R. E., Masters, C. L., Arch. Neurol., 2003, 60, 1685-1691
Ritche, C. W., Bush, A. I., Masters, C. I., Expert Opin. Invest. Drugs, 2004, 13, 1585-1592
Salvador, G. A., Uranga, R. M., Giusto, N. M., Int. J. Alzheimers Dis., 2010, 2011
Scott, L. E., Orvig, C., Chem. Rev., 2009, 109, 4885-4910
Selkoe, D. J., Neuron, 2001, 32, 177-180
Shearer, J., Szalai, V. A., J. Am. Chem. Soc., 2008, 130, 17826-17835
Shinozaki, Y., Koizumi, S., Ishida, S., Sawada, J., Ohno, Y., Inoue, K., Glia, 2005, 49(2), 288-300
Sigel, H., Griesser, R., Chem. Soc. Rev., 2005, 34, 875-900
Simons, M., Schwairzler, F., Litjohann, D., Bergmann, K. V., Beyreuther, K., Dichgans, J., Wormstall, H., Hartmann, T., Schulz, J. B., Ann. Neurol., 2002, 52, 346-350
Singh, A. N., Newborn, J. S., Raushel, F. M., Bioorganic Chemistry, 1988, 16(2), 206-214
Soscia, S. J., Kirby, J. E., Washicosky, K. J., Stephanie, T., Ingelsson, M. M., Hyman, B., Burton, M. A., Goldstein, L. E., Duong, S., Tanzi, R. E., Moir, R. D., PLoS One, 2010, 5, e9505
Stern, N., Major, D. T., Gottlieb, H. E., Weizman, D., Fischer, B., Org. Biomol. Chem., 2010, 8, 4637
Storr, T., Scott, L. E., Bowen, M. L., Green, D. E., Thompson, K. H., Schugar, H. J., Orvig, C., Dalton Trans., 2009, 3034-3043
Toyota, K., Ishikawa, Y., Shirabe, K., Yoshifuji, M., Okada, K., Hirotsu, K., Heteroat. Chem., 1993, 4, 279-285
Tulapurkar, M. E., Kaubinger, W., Nahum, V., Fischer, B., Reiser, G., British Journal of Pharmacology, 2004, 142(5), 869-878
Tulapurkar, M. E., Zundorf, G., Reiser, G., Journal of Neurochemistry, 2006, 96(3), 624-634
Ubl, J. J., Vöhringer, C., Reiser, G., Neuroscience (Oxford), 1998, 86(2), 597-609
Weaver, J., Pollack, S., Biochem. J., 1989, 261, 787-792
Weaver, J., Zhan, H., Pollack, S., Br. J. Haematol., 1993, 83, 138-144
White, A. R., Barnham, K. J., Huang, X., Voltakis, I., Beyreuther, K., Masters, C. L., Cherny, R. A., Bush, A. I., Cappai, R., JBIC, J. Biol. Inorg. Chem., 2004, 9, 269-280
Yatsunyk, L. A., Rosenzweig, A. C., J. Biol. Chem., 2007, 282, 8622-8631
Zlokovic, B. V., J. Neurochem., 2004, 89, 807-811
Zylberg, J., Ecke, D., Fischer, B., Reiser, G., Biochemical Journal, 2007, 405(2), 277-286

1. A nucleoside 5′-phosphorothioate of the general formula I: 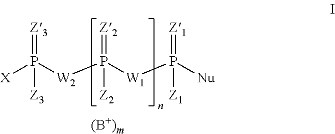
or a diastereomer or mixture of diastereomers thereof,
wherein
X is —O⁻, a glucose moiety linked through the oxygen atom linked to its 1- or 6-position, or a group of the formula —O—CH₂—OC(O)—R₁₂ or —NH—(CHR₁₃)—C(O)—OR₁₃;
Nu is an adenosine residue of the formula Ia, linked through the oxygen atom linked to the 5′-position: 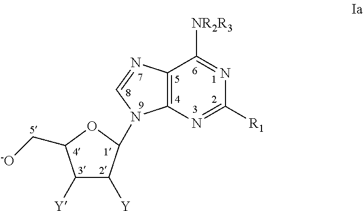
wherein
R₁ is H, halogen, —O-hydrocarbyl, —S-hydrocarbyl, —NR₄R₅, heteroaryl, or hydrocarbyl optionally substituted by one or more groups each independently selected from the group consisting of halogen, —CN, —SCN, —NO₂, —OR₄, —SR₄, —NR₄R₅ and heteroaryl, wherein R₄ and R₅ each independently is H or hydrocarbyl, or R₄ and R₅ together with the nitrogen atom to which they are attached form a saturated or unsaturated heterocyclic ring optionally containing 1-2 further heteroatoms selected from the group consisting of N, O and S, wherein the additional nitrogen is optionally substituted by alkyl; and
R₂ and R₃ each independently is H or hydrocarbyl;
or an uridine residue of the formula Ib, linked through the oxygen atom linked to the 5′-position: 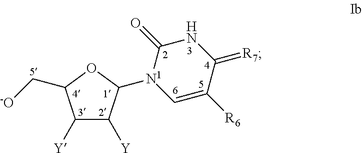
wherein
R₆ is H, halogen, —O-hydrocarbyl, —S-hydrocarbyl, —NR₈R₉, heteroaryl, or hydrocarbyl optionally substituted by one or more groups each independently selected from the group consisting of halogen, —CN, —SCN, —NO₂, —OR₈, —SR₈, —NR₈R₉ and heteroaryl, wherein R₈ and R₉ each independently is H or hydrocarbyl, or R₈ and R₉ together with the nitrogen atom to which they are attached form a saturated or unsaturated heterocyclic ring optionally containing 1-2 further heteroatoms selected from the group consisting of N, O and S, wherein the additional nitrogen is optionally substituted by alkyl; and
R₇ is O or S;
Y and Y′ each independently is H, —OH or —NH₂;
W₁ and W₂ each independently is —O—, —NH— or —C(R₁₀R₁₁)—, wherein R₁₀ and R₁₁ each independently is H or halogen;
Z₁, Z′₁, Z₂, Z′₂ and Z′₃ each independently is O, —O⁻, S, —S⁻ or —BH₃⁻;
Z₃ is —O⁻, —S⁻, —BH₃⁻, or a group of the formula —O—CH₂—OC(O)—R₁₂ or —NH—(CHR₁₃)—C(O)—OR₁₃;
R₁₂ is (C₁-C₄)alkyl;
R₁₃ each independently is (C₁-C₄)alkyl, (C₆-C₁₀)aryl or (C₆-C₁₀)aryl-(C₁-C₄)alkyl;
n is 0 or 1;
m is 2, 3 or 4; and
B⁺ represents a pharmaceutically acceptable cation,
provided that (i) at least one of W₁ and W₂ is not —O—, and at least one of Z₁, Z′₁, Z₂, Z′₂, Z₃ and Z′₃ is S or —S⁻; and (ii) when X is a glucose moiety, Z₃ is —O⁻, —S⁻, or —BH₃⁻; and when X is a group of the formula —O—CH₂—OC(O)—R₁₂ or —NH—(CHR₁₃)—C(O)—OR₁₃, Z₃ is a group of the formula —O—CH₂—OC(O)—R₁₂ or —NH—(CHR₁₃)—C(O)—OR₁₃, respectively.

2. The nucleoside 5′-phosphorothioate of claim 1, wherein Nu is an adenosine residue of the formula Ia, wherein R₁ is H, halogen, —O-hydrocarbyl, —S-hydrocarbyl, —NR₄R₅, heteroaryl, or hydrocarbyl; R₄ and R₅ each independently is H or hydrocarbyl, or R₄ and R₅ together with the nitrogen atom to which they are attached form a 5- or 6-membered saturated or unsaturated heterocyclic ring optionally containing 1-2 further heteroatoms selected from N, O or S; said hydrocarbyl each independently is (C₁-C₈)alkyl, (C₂-C₈)alkenyl, (C₂-C₈)alkynyl, or (C₆-C₁₄)aryl; and said heteroaryl is a 5-6-membered monocyclic heteroaromatic ring containing 1-2 heteroatoms selected from the group consisting of N, O and S.

3. The nucleoside 5′-phosphorothioate of claim 2, wherein R₁ is H, —O-hydrocarbyl, —S-hydrocarbyl, —NR₄R₅, or hydrocarbyl; R₄ and R₅ each independently is H or hydrocarbyl; and said hydrocarbyl each independently is (C₁-C₄)alkyl, (C₂-C₄)alkenyl, (C₂-C₄)alkynyl, or (C₆-C₁₀)aryl.

4. The nucleoside 5′-phosphorothioate of claim 3, wherein said hydrocarbyl each independently is methyl or ethyl.

5. The nucleoside 5′-phosphorothioate of claim 1, wherein Nu is an adenosine residue of the formula Ia, wherein R₂ and R₃ each independently is H or hydrocarbyl; and said hydrocarbyl is (C₁-C₄)alkyl, (C₂-C₄)alkenyl, (C₂-C₄)alkynyl, or (C₆-C₁₀)aryl.

6. The nucleoside 5′-phosphorothioate of claim 1, wherein Nu is an adenosine residue of the formula Ia, wherein R₁, R₂ and R₃ are H.

7. The nucleoside 5′-phosphorothioate of claim 1, wherein Nu is an uridine residue of the formula Ib, wherein R₆ is H, halogen, —O-hydrocarbyl, —S-hydrocarbyl, —NR₈R₉, heteroaryl, or hydrocarbyl; R₈ and R₉ each independently is H or hydrocarbyl, or R₈ and R₉ together with the nitrogen atom to which they are attached form a 5- or 6-membered saturated or unsaturated heterocyclic ring optionally containing 1-2 further heteroatoms selected from the group consisting of N, O and S; said hydrocarbyl each independently is (C₁-C₈)alkyl, (C₂-C₈)alkenyl, (C₂-C₈)alkynyl, or (C₆-C₁₄)aryl; and said heteroaryl is a 5-6-membered monocyclic heteroaromatic ring containing 1-2 heteroatoms selected from the group consisting of N, O and S.

8. The nucleoside 5′-phosphorothioate of claim 7, wherein R₆ is H, —O-hydrocarbyl, —S-hydrocarbyl, —NR₈R₉, or hydrocarbyl; R₈ and R₉ each independently is H or hydrocarbyl; and said hydrocarbyl each independently is (C₁-C₄)alkyl, (C₂-C₄)alkenyl, (C₂-C₄)alkynyl, or (C₆-C₁₀)aryl.

9. The nucleoside 5′-phosphorothioate of claim 8, wherein said hydrocarbyl each independently is methyl or ethyl.

10. The nucleoside 5′-phosphorothioate of claim 1, wherein Nu is an uridine residue of the formula Ib, wherein R₇ is O.

11. The nucleoside 5′-phosphorothioate of claim 1, wherein Nu is an uridine residue of the formula Ib, wherein R₆ is H; and R₇ is O.

12. The nucleoside 5′-phosphorothioate of claim 1, wherein Y′ is —OH; and Y is H or —OH.

13. The nucleoside 5′-phosphorothioate of claim 1, wherein W₁ and W₂ each independently is —O— or —C(R₁₀R₁₁)—, wherein R₁₀ and R₁₁ each independently is H, Cl or F.

14. The nucleoside 5′-phosphorothioate of claim 1, wherein X is —O⁻, or a glucose moiety.

15. The nucleoside 5′-phosphorothioate of claim 14, wherein n is 0, W₂ is —C(R₁₀R₁₁)—, and:
(i) one of Z₁ and Z′₁ is —S⁻ or S, and another of Z₁ and Z′₁, Z₃ and Z′₃ each independently is O or —O⁻; or one of Z₃ and Z′₃ is —S⁻ or S, and Z₁, Z′₁, and another of Z₃ and Z′₃, each independently is O or —O⁻;
(ii) one of Z₁ and Z′₁, and one of Z₃ and Z′₃, each independently is —S⁻ or S, and the other of Z₁, Z′₁, Z₃ and Z′₃ each independently is O or —O⁻; Z₁ and Z′₁ each independently is —S⁻ or S, and Z₃ and Z′₃ each independently is O or —O⁻; or Z₃ and Z′₃ each independently is —S⁻ or S, and Z₁ and Z′₁ each independently is O or —O⁻;
(iii) Z₁, Z′₁, and one of Z₃ and Z′₃, each independently is —S⁻ or S, and another of Z₃ and Z′₃ is O or —O⁻; or Z₃, Z′₃, and one of Z₁ and Z′₁, each independently is —S⁻ or S, and another of Z₁ and Z′₁ is O or —O⁻; or
(iv) Z₁, Z′₁, Z₃ and Z′₃ each independently is —S⁻ or S.

16. The nucleoside 5′-phosphorothioate of claim 14, wherein n is 1, either one of W₁ and W₂ is —O— and another of W₁ and W₂ is —C(R₁₀R₁₁)—, or both W₁ and W₂ each independently is —C(R₁₀R₁₁)—, and:
(i) one of Z₁ and Z′₁ is —S⁻ or S, and another of Z₁ and Z′₁, Z₂, Z′₂, Z₃ and Z′₃ each independently is O or —O⁻; one of Z₂ and Z′₂ is —S⁻ or S, and Z₁, Z′₁, another of Z₂ and Z′₂, Z₃ and Z′₃ each independently is O or —O⁻; or one of Z₃ and Z′₃ is —S⁻ or S, and Z₁, Z′₁, Z₂, Z′₂, and another of Z₃ and Z′₃, each independently is O or —O⁻;
(ii) one of Z₁ and Z′₁, and one of Z₂ and Z′₂, each independently is —S⁻ or S, and the other of Z₁, Z′₁, Z₂, Z′₂, and Z₃ and Z′₃, each independently is O or —O⁻; one of Z₁ and Z′₁, and one of Z₃ and Z′₃, each independently is —S⁻ or S, and the other of Z₁, Z′₁, Z₃, Z′₃, and Z₂ and Z′₂, each independently is O or —O⁻; one of Z₂ and Z′₂, and one of Z₃ and Z′₃, each independently is —S⁻ or S, and Z₁, Z′₁, and the other of Z₂, Z′₂, Z₃, Z′₃, each independently is O or —O⁻; Z₁ and Z′₁ each independently is —S⁻ or S, and Z₂, Z′₂, Z₃ and Z′₃ each independently is O or —O⁻; Z₂ and Z′₂ each independently is —S⁻ or S, and Z₁, Z′₁, Z₃ and Z′₃ are O or —O⁻; or Z₃ and Z′₃ each independently is —S⁻ or S, and Z₁, Z′₁, Z₂ and Z′₂ are O or —O⁻;
(iii) one of Z₁ and Z′₁, one of Z₂ and Z′₂, and one of Z₃ and Z′₃, each independently is —S⁻ or S, and the other of Z₁, Z′₁, Z₂, Z′₂, Z₃ and Z′₃ each independently is O or —O⁻; Z₁ and Z′₁, and one of Z₂ and Z′₂, each independently is —S⁻ or S, and another of Z₂ and Z′₂, Z₃ and Z′₃ each independently is O or —O⁻; Z₁ and Z′₁, and one of Z₃ and Z′₃, each independently is —S⁻ or S, and Z₂, Z′₂, and another of Z₃ and Z′₃ each independently is O or —O⁻; Z₂ and Z′₂, and one of Z₁ and Z′₁, each independently is —S⁻ or S, and another of Z₁ and Z′₁, Z₃ and Z′₃ each independently is O or —O⁻; Z₂ and Z′₂, and one of Z₃ and Z′₃, each independently is —S⁻ or S, and Z₁, Z′₁, and another of Z₃ and Z′₃, each independently is O or —O⁻; Z₃ and Z′₃, and one of Z₁ and Z′₁, each independently is —S⁻ or S, and another of Z₁ and Z′₁, Z₂ and Z′₂ each independently is O or —O⁻; or Z₃ and Z′₃, and one of Z₂ and Z′₂, each independently is —S⁻ or S, and Z₁, Z′₁, and another of Z₂ and Z′₂, each independently is O or —O⁻;
(iv) Z₁, Z′₁, one of Z₂ and Z′₂, and one of Z₃ and Z′₃, each independently is —S⁻ or S, and the other of Z₂, Z′₂, Z₃ and Z′₃ each independently is O or —O⁻; Z₂, Z′₂, one of Z₁ and Z′₁, and one of Z₃ and Z′₃, each independently is —S⁻ or S, and the other of Z₁, Z′₁, Z₃ and Z′₃ each independently is O or —O⁻; Z₃, Z′₃, one of Z₁ and Z′₁, and one of Z₂ and Z′₂, each independently is —S⁻ or S, and the other of Z₁, Z′₁, Z₂ and Z′₂ each independently is O or —O⁻; Z₁, Z′₁, Z₂ and Z′₂ each independently is —S⁻ or S, and Z₃ and Z′₃ each independently is O or —O⁻; Z₁, Z′₁, Z₃ and Z′₃ each independently is —S⁻ or S, and Z₂ and Z′₂ each independently is O or —O⁻; or Z₂, Z′₂, Z₃ and Z′₃ each independently is —S⁻ or S, and Z₁ and Z′₁ each independently is O or —O⁻;
(v) Z₁, Z′₁, Z₂, Z′₂, and one of Z₃ and Z′₃, each independently is —S⁻ or S, and another of Z₃ and Z′₃ is O or —O⁻; Z₁, Z′₁, Z₃, Z′₃, and one of Z₂ and Z′₂, each independently is —S⁻ or S, and another of Z₂ and Z′₂ is O or —O⁻; or Z₂, Z′₂, Z₃, Z′₃, and one of Z₁ and Z′₁, each independently is —S⁻ or S, and another of Z₁ and Z′₁ is O or —O⁻; or
(vi) Z₁, Z′₁, Z₂, Z′₂, Z₃ and Z′₃ each independently is —S⁻ or S.

17. The nucleoside 5′-phosphorothioate of claim 15, wherein X is —O⁻, or a glucose moiety; Y and Y′ are —OH; n is 0; W₂ is —CH₂—, —CCl₂— or —CF₂—; and Nu is (i) an adenosine residue of the formula Ia, wherein R₁, R₂ and R₃ are H; or (ii) an uridine residue of the formula Ib, wherein R₆ is H; and R₇ is O.

18. The nucleoside 5′-phosphorothioate of claim 17, wherein X is —O⁻; Nu is an adenosine residue of the formula Ia, wherein R₁, R₂ and R₃ are H, or an uridine residue of the formula Ib, wherein R₆ is H, and R₇ is O; Y and Y′ are —OH; n is 0; W₂ is —CH₂—; and Z₁, Z′₁, Z₃ and Z′₃ are —S⁻ or S; or

19. The nucleoside 5′-phosphorothioate of claim 16, wherein X is —O⁻, or a glucose moiety; Y and Y′ are —OH; n is 1; either one of W₁ and W₂ is —O— and another of W₁ and W₂ is —CH₂—, —CCl₂— or —CF₂—, or both W₁ and W₂ are —CH₂—, —CCl₂— or —CF₂—; and Nu is (i) an adenosine residue of the formula Ia, wherein R₁, R₂ and R₃ are H; or (ii) an uridine residue of the formula Ib, wherein R₆ is H, and R₇ is O.

20. The nucleoside 5′-phosphorothioate of claim 19, wherein:
(i) X is —O⁻; Nu is an adenosine residue of the formula Ia, wherein R₁, R₂ and R₃ are H; Y and Y′ are —OH; n is 1; W₁ is —CH₂—; W₂ is —O⁻; and one of Z₃ and Z′₃ is —S⁻ or S, and Z₁, Z′₁, Z₂, Z′₂, and another of Z₃ and Z′₃, are O or —O⁻;
(ii) X is —O⁻; Nu is an adenosine residue of the formula Ia, wherein R₁, R₂ and R₃ are H; Y and Y′ are —OH; n is 1; W₁ is —O⁻; W₂ is —CH₂—; and one of Z₁ and Z′₁ is —S⁻ or S, and another of Z₁ and Z′₁, Z₂, Z′₂, Z₃ and Z′₃ are O or —O⁻;
(iii) X is —O⁻; Nu is an adenosine residue of the formula Ia, wherein R₁, R₂ and R₃ are H; Y and Y′ are —OH; n is 1; W₁ is —O⁻; W₂ is —CCl₂—; and one of Z₁ and Z′₁ is —S⁻ or S, and another of Z₁ and Z′₁, Z₂, Z′₂, Z₃ and Z′₃ are O or —O⁻; or
(iv) X is a glucose moiety linked through the oxygen atom linked to its 1-position; Nu is an adenosine residue of the formula Ia, wherein R₁, R₂ and R₃ are H; Y and Y′ are —OH; n is 1; W₁ is —CH₂—; W₂ is —O⁻; and one of Z₃ and Z′₃ is —S⁻ or S, and Z₁, Z′₁, Z₂, Z′₂, and another of Z₃ and Z′₃, are O or —O⁻.

21. The nucleoside 5′-phosphorothioate of claim 19(iii), characterized by being the isomer with a retention time (Rt) of 20.3 min when separated from a mixture of diastereoisomers using a semi-preparative reverse-phase Gemini 5u column (C-18 110A, 250×10 mm, 5 μm), and gradient elution from 96.5:3.5 to 95.5:4.5 [100 mM triethylammonium acetate, pH 7:CH₃CN] over 31 min at a flow rate of 4.5 ml/min.

22. The nucleoside 5′-phosphorothioate of claim 1, wherein X is a group of the formula —O—CH₂—OC(O)—R₁₂ or —NH—(CHR₁₃)—C(O)—OR₁₃.

23. The nucleoside 5′-phosphorothioate of claim 22, wherein n is 0, W₂ is —C(R₁₀R₁₁)—, and:
(i) one of Z₁ and Z′₁ is —S⁻ or S, and another of Z₁ and Z′₁, and Z′₃ each independently is O or —O⁻; or Z′₃ is —S⁻ or S, and Z₁ and Z′₁ each independently is O or —O⁻;
(ii) one of Z₁ and Z′₁, and Z′₃, each independently is —S⁻ or S, and the other of Z₁ and Z′₁ is O or —O⁻; or Z₁ and Z′₁ each independently is —S⁻ or S, and Z′₃ is O or —O⁻; or
(iii) Z₁, Z′₁ and Z′₃ each independently is —S⁻ or S.

24. The nucleoside 5′-phosphorothioate of claim 22, wherein n is 1, either one of W₁ and W₂ is —O— and another of W₁ and W₂ is —C(R₁₀R₁₁)—, or both W₁ and W₂ each independently is —C(R₁₀R₁₁)—, and:
(i) one of Z₁ and Z′₁ is —S⁻ or S, and another of Z₁ and Z′₁, Z₂, Z′₂ and Z′₃ each independently is O or —O⁻; one of Z₂ and Z′₂ is —S⁻ or S, and Z₁, Z′₁, another of Z₂ and Z′₂ and Z′₃ each independently is O or —O⁻; or Z′₃ is —S⁻ or S, and Z₁, Z′₁, Z₂ and Z′₂ each independently is O or —O⁻;
(ii) one of Z₁ and Z′₁, and one of Z₂ and Z′₂, each independently is —S⁻ or S, and the other of Z₁, Z′₁, Z₂, Z′₂, and Z′₃, each independently is O or —O⁻; one of Z₁ and Z′₁, and Z′₃, each independently is —S⁻ or S, and the other of Z₁ and Z′₁, and Z₂ and Z′₂, each independently is O or —O⁻; one of Z₂ and Z′₂, and Z′₃, each independently is —S⁻ or S, and Z₁, Z′₁, and the other of Z₂ and Z′₂, each independently is O or —O⁻; Z₁ and Z′₁ each independently is —S⁻ or S, and Z₂, Z′₂ and Z′₃ each independently is O or —O⁻; or Z₂ and Z′₂ each independently is —S⁻ or S, and Z₁, Z′₁ and Z′₃ each independently is O or —O⁻;
(iii) one of Z₁ and Z′₁, one of Z₂ and Z′₂, and Z′₃, each independently is —S⁻ or S, and the other of Z₁, Z′₁, Z₂ and Z′₂ each independently is O or —O⁻; Z₁ and Z′₁, and one of Z₂ and Z′₂, each independently is —S⁻ or S, and another of Z₂ and Z′₂, and Z′₃ each independently is O or —O⁻; Z₁ and Z′₁, and Z′₃, each independently is —S⁻ or S, and Z₂, Z′₂ are O or —O⁻; Z₂ and Z′₂, and one of Z₁ and Z′₁, each independently is —S⁻ or S, and another of Z₁ and Z′₁, and Z′₃ each independently is O or —O⁻; or Z₂ and Z′₂, and Z′₃, each independently is —S⁻ or S, and Z₁ and Z′₁ each independently is O or —O⁻;
(iv) Z₁, Z′₁, one of Z₂ and Z′₂, and Z′₃, each independently is —S⁻ or S, and the other of Z₂ and Z′₂ is O or —O⁻; Z₂, Z′₂, one of Z₁ and Z′₁, and Z′₃, each independently is —S⁻ or S, and the other of Z₁ and Z′₁ is O or —O⁻; or Z₁, Z′₁, Z₂ and Z′₂ each independently is —S⁻ or S, and Z′₃ is O or —O⁻; or
(v) Z₁, Z′₁, Z₂, Z′₂, and Z′₃ each independently is —S⁻ or S.

25. The nucleoside 5′-phosphorothioate of claim 1, wherein B is a cation of an alkali metal, NH₄⁺, an organic cation of the formula R₄N⁺ wherein each one of the Rs independently is H or C₁-C₂₂ alkyl, a cationic lipid or a mixture of cationic lipids.

26. A pharmaceutical composition comprising a nucleoside 5′-phosphorothioate of the general formula I as claimed in claim 1, and a pharmaceutically acceptable carrier or diluent.

27. The pharmaceutical composition of claim 26, wherein said nucleoside 5′-phosphorothioate is a compound of the general formula I, wherein X is —O⁻; Nu is an adenosine residue of the formula Ia, wherein R₁, R₂ and R₃ are H; Y and Y′ are —OH; n is 1; and (i) W₁ is —CH₂—; W₂ is —O⁻; and one of Z₃ and Z′₃ is —S⁻ or S, and Z₁, Z′₁, Z₂, Z′₂, and another of Z₃ and Z′₃, are O or —O⁻; (ii) W₁ is —O⁻; W₂ is —CH₂—; and one of Z₁ and Z′₁ is —S⁻ or S, and another of Z₁ and Z′₁, Z₂, Z′₂, Z₃ and Z′₃ are O or —O⁻; or (iii) W₁ is —O⁻; W₂ is —CCl₂—; and one of Z₁ and Z′₁ is —S⁻ or S, and another of Z₁ and Z′₁, Z₂, Z′₂, Z₃ and Z′₃ are O or —O⁻.

28. The pharmaceutical composition of claim 26, wherein the composition is configured for intravenous, intraarterial, intramuscular, intraperitoneal, intrathecal, intrapleural, intratracheal, subcutaneous, transdermal, sublingual, inhalational, or oral administration.
